SEQUENCE LISTING (72 sequences)

SEQ ID NO: 1 - DNA, 1024 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggta

SEQ ID NO: 2 - DNA, 27 bp, Artificial Sequence
  Description of Artificial Sequence Synthetic primer
atgtccgagcttaatgaaaagttagcc

SEQ ID NO: 3 - DNA, 34 bp, Artificial Sequence
  Description of Artificial Sequence Synthetic primer
ttacatagattgagtgaaggtacgagtaataacg

SEQ ID NO: 4 - DNA, 741 bp, Escherichia coli
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 5 - DNA, 26 bp, Artificial Sequence
  Description of Artificial Sequence Synthetic primer
atgtcagttattggtcgcattcactc

SEQ ID NO: 6 - DNA, 30 bp, Artificial Sequence
  Description of Artificial Sequence Synthetic primer
ttagaacattaccttatgaccgtactgctc

SEQ ID NO: 7 - DNA, 1024 bp, Escherichia coli
atgaaggtagatattgataccagcgataagctgtacgccgacgcatggcttggctttaaaggtacggactggaaaaacgaaattaatgtccgcgattttattcaacataactatacaccgtatgaaggcgatgaatctttcctcgccgaagcgacgcctgccaccacggaattgtgggaaaaagtaatggaaggcatccgtatcgaaaatgcaacccacgcgccggttgatttcgataccaatattgccaccacaattaccgctcatgatgcgggatatattaaccagccgctggaaaaaattgttggcctgcaaacggatgcgccgttgaaacgtgcgctacacccgttcggtggcattaatatgattaaaagttcattccacgcctatggccgagaaatggacagtgaatttgaatatctgtttaccgatctgcgtaaaacccataaccagggcgtatttgatgtttactcaccggatatgctgcgctgccgtaaatctggcgtgctgaccggtttaccagatggctatggccgtgggcgcattatcggtgactatcgccgcgtagcgctgtatggcatcagttatctggtacgtgaacgcgaactgcaatttgccgatctccagtctcgtctggaaaaaggcgaggatctggaagccaccatccgtctgcgtgaggagctggcagagcatcgtcatgcgctgttgcagattcaggaaatggcggcgaaatatggctttgatatctctcgcccggcgcagaatgcgcaggaagcggtgcagtggctctacttcgcttatctggcggcagtgaaatcgcaaaatggcggcgcgatgtcgctgggccgcacggcatcgttcctcgatatctacattgagcgcgactttaaagctggcgtactcaatgagcagcaggcacaggaactgatcgatcacttcatcatgaagatccgtatggtacgcttcctgcgtacaccggaatttgattcgctgttctccggcgacccaatctgggcgacggaag

SEQ ID NO: 8 - DNA, 31 bp, Artificial Sequence
  Description of Artificial Sequence Synthetic primer
atgaaggtagatattgataccagcgataagc

SEQ ID NO: 9 - DNA, 24 bp, Artificial Sequence
  Description of Artificial Sequence Synthetic primer
ttagagcgcctgggtaaaggtacg

SEQ ID NO: 10 - DNA, 384 bp, Escherichia coli
atgattacaggtatccagattactaaagccgctaacgacgatctgctgaactctttctggctgctggacagcgaaaaaggcgaagcgcgttgcatcgttgcaaaagcaggttatgcagaagatgaagtggttgcagtaagcaaactgggtgacattgaataccgtgaagttccagtagaagtgaaaccagaagttcgcgttgaaggtggtcaacacctgaacgttaacgttctgcgtcgcgaaactctggaagatgcagttaagcatccggaaaaatatccgcagctgaccatccgtgtatccggttatgcagttcgctttaactctctgactccggaacagcagcgcgacgttatcgctcgtacctttactgaaagcctgtaa

SEQ ID NO: 11 - DNA, 31 bp, Artificial Sequence
  Description of Artificial Sequence Synthetic primer
atgattacaggtatccagattactaaagccg

SEQ ID NO: 12 - DNA, 27 bp, Artificial Sequence
  Description of Artificial Sequence Synthetic primer
ttacaggctttcagtaaaggtacgagc

SEQ ID NO: 13 - DNA, 1024 bp, Escherichia coli
atgtctaacgtgcaggagtggcaacagcttgccaacaaggaattgagccgtcgggagaaaactgtcgactcgctggttcatcaaaccgcggaagggatcgccatcaagccgctgtataccgaagccgatctcgataatctggaggtgacaggtacccttcctggtttgccgccctacgttcgtggcccgcgtgccactatgtataccgcccaaccgtggaccatccgtcagtatgctggtttttcaacagcaaaagagtccaacgctttttatcgccgtaacctggccgccgggcaaaaaggtctttccgttgcgtttgaccttgccacccaccgtggctacgactccgataacccgcgcgtggcgggcgacgtcggcaaagcgggcgtcgctatcgacaccgtggaagatatgaaagtcctgttcgaccagatcccgctggataaaatgtcggtttcgatgaccatgaatggcgcagtgctaccagtactggcgttttatatcgtcgccgcagaagagcaaggtgttacacctgataaactgaccggcaccattcaaaacgatattctcaaagagtacctctgccgcaacacctatatttacccaccaaaaccgtcaatgcgcattatcgccgacatcatcgcctggtgttccggcaacatgccgcgatttaataccatcagtatcagcggttaccacatgggtgaagcgggtgccaactgcgtgcagcaggtagcatttacgctcgctgatgggattgagtacatcaaagcagcaatctctgccggactgaaaattgatgacttcgctcctcgcctgtcgttcttcttcggcatcggcatggatctgtttatgaacgtcgccatgttgcgtgcggcacgttatttatggagcgaagcggtcagtggatttggcgcacaggacccgaaatcactggcgctgcgtacccactgccagacctcaggctggagcctgactgaacaggatccgtataacaacgttatccgcaccaccattgaagcgc

SEQ ID NO: 14 - DNA, 25 bp, Artificial Sequence
  Description of Artificial Sequence Synthetic primer
atgtctaacgtgcaggagtggcaac

SEQ ID NO: 15 - DNA, 30 bp, Artificial Sequence
  Description of Artificial Sequence Synthetic primer
ttaatcatgatgctggcttatcagattcag

SEQ ID NO: 16 - DNA, 996 bp, Escherichia coli
atgattaatgaagccacgctggcagaaagtattcgccgcttacgtcagggtgagcgtgccacactcgcccaggccatgacgctggtggaaagccgtcacccgcgtcatcaggcactaagtacgcagctgcttgatgccattatgccgtactgcggtaacaccctgcgactgggcgttaccggcacccccggcgcggggaaaagtacctttcttgaggcctttggcatgttgttgattcgagagggattaaaggtcgcggttattgcggtcgatcccagcagcccggtcactggcggtagcattctcggggataaaacccgcatgaatgacctggcgcgtgccgaagcggcgtttattcgcccggtaccatcctccggtcatctgggcggtgccagtcagcgagcgcgggaattaatgctgttatgcgaagcagcgggttatgacgtagtgattgtcgaaacggttggcgtcgggcagtcggaaacagaagtcgcccgcatggtggactgttttatctcgttgcaaattgccggtggcggcgatgatctgcagggcattaaaaaagggctgatggaagtggctgatctgatcgttatcaacaaagacgatggcgataaccataccaatgtcgccattgcccggcatatgtacgagagtgccctgcatattctgcgacgtaaatacgacgaatggcagccacgggttctgacttgtagcgcactggaaaaacgtggaatcgatgagatctggcacgccatcatcgacttcaaaaccgcgctaactgccagtggtcgtttacaacaagtgcggcaacaacaatcggtggaatggctgcgtaagcagaccgaagaagaagtactgaatcacctgttcgcgaatgaagatttcgatcgctattaccgccagacgcttttagcggtcaaaaacaatacgctctcaccgcgcaccggcctgcggcagctcagtgaatttatccagacgcaatattttgattaa

SEQ ID NO: 17 - DNA, 786 bp, Escherichia coli
atgtcttatcagtatgttaacgttgtcactatcaacaaagtggcggtcattgagtttaactatggccgaaaacttaatgccttaagtaaagtctttattgatgatcttatgcaggcgttaagcgatctcaaccggccggaaattcgctgtatcattttgcgcgcaccgagtggatccaaagtcttctccgcaggtcacgatattcacgaactgccgtctggcggtcgcgatccgctctcctatgatgatccattgcgtcaaatcacccgcatgatccaaaaattcccgaaaccgatcatttcgatggtggaaggtagtgtttggggtggcgcatttgaaatgatcatgagttccgatctgatcatcgccgccagtacctcaaccttctcaatgacgcctgtaaacctcggcgtcccgtataacctggtcggcattcacaacctgacccgcgacgcgggcttccacattgtcaaagagctgatttttaccgcttcgccaatcaccgcccagcgcgcgctggctgtcggcatcctcaaccatgttgtggaagtggaagaactggaagatttcaccttacaaatggcgcaccacatctctgagaaagcgccgttagccattgccgttatcaaagaagagctgcgtgtactgggcgaagcacacaccatgaactccgatgaatttgaacgtattcaggggatgcgccgcgcggtgtatgacagcgaagattaccaggaagggatgaacgctttcctcgaaaaacgtaaacctaatttcgttggtcattaa

SEQ ID NO: 18 - DNA, 33 bp, Artificial Sequence
  Description of Artificial Sequence Synthetic primer
atgtcttatcagtatgttaacgttgtcactatc

SEQ ID NO: 19 - DNA, 27 bp, Artificial Sequence
  Description of Artificial Sequence Synthetic primer
ttaatgaccaacgaaattaggtttacg

SEQ ID NO: 20 - DNA, 1024 bp, Ralstonia eutropha
atgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccga

SEQ ID NO: 21 - DNA, 26 bp, Artificial Sequence
  Description of Artificial Sequence Synthetic primer
atgacgcgtgaagtggtagtggtaag

SEQ ID NO: 22 - DNA, 23 bp, Artificial Sequence
  Description of Artificial Sequence Synthetic primer
ttagatacgctcgaagatggcgg

SEQ ID NO: 23 - DNA, 1024 bp, Acinetobacter sp.
atgaaagatgttgtgattgttgcagcaaaacgtactgcgattggtagctttttaggtagtcttgcatctttatctgcaccacagttggggcaaacagcaattcgtgcagttttagacagcgctaatgtaaaacctgaacaagttgatcaggtgattatgggcaacgtactcacgacaggcgtgggacaaaaccctgcacgtcaggcagcaattgctgctggtattccagtacaagtgcctgcatctacgctgaatgtcgtctgtggttcaggtttgcgtgcggtacatttggcagcacaagccattcaatgcgatgaagccgacattgtggtcgcaggtggtcaagaatctatgtcacaaagtgcgcactatatgcagctgcgtaatgggcaaaaaatgggtaatgcacaattggtggatagcatggtggctgatggtttaaccgatgcctataaccagtatcaaatgggtattaccgcagaaaatattgtagaaaaactgggtttaaaccgtgaagaacaagatcaacttgcattgacttcacaacaacgtgctgcggcagctcaggcagctggcaagtttaaagatgaaattgccgtagtcagcattccacaacgtaaaggtgagcctgttgtatttgctgaagatgaatacattaaagccaataccagccttgaaagcctcacaaaactacgcccagcctttaaaaaagatggtagcgtaaccgcaggtaatgcttcaggcattaatgatggtgcagcagcagtactgatgatgagtgcggacaaagcagcagaattaggtcttaagccattggcacgtattaaaggctatgccatgtctggtattgagcctgaaattatggggcttggtcctgtcgatgcagtaaagaaaaccctcaacaaagcaggctggagcttagatcaggttgatttgattgaagccaatgaagcatttgctgcacaggctttgggtgttgctaaagaattaggcttagacctggataaagtcaacgtcaatggcg

SEQ ID NO: 24 - DNA, 26 bp, Artificial Sequence
  Description of Artificial Sequence Synthetic primer
atgaaagatgttgtgattgttgcagc

SEQ ID NO: 25 - DNA, 27 bp, Artificial Sequence
  Description of Artificial Sequence Synthetic primer
ttagtcacgttcaactgcaagtgcaac

SEQ ID NO: 26 - DNA, 699 bp, Helicobacter pylori
atgaacaaggttataaccgatttagacaaagcattgagcgggttaaaagacggggacactattttagtgggcggttttgggctgtgcgggatacccgaatacgccattaattacatttataagaaaggcattaaggatttgattgtcgtgagcaataattgcggcgttgatgactttgggttgggcattcttttagaaaaaaaacagattaaaaagattatcgcttcctatgtgggagaaaataagatttttgaatcgcaaatgctgaacggagaaattgaagtcgttttgacaccgcaaggcacgctagctgaaaacttgcgcgctggaggggctgggatacccgcttactacaccccaaccggtgttgggactttgatcgctcaaggcaaggaatcaagggagtttaacggcaaagagtatattttagaaagagcgatcacaggcgattacgggcttatcaaagcctataaaagcgatactttagggaatttggtgttcagaaagacagccaggaatttcaatcccttgtgcgcgatggcggcaaaaatatgcgtcgctgaagtggaagaaattgtcccggccggggaattagacccagatgaaatacacttgccaggaatctatgtgcaacacatctataagggcgagaaatttgaaaaacggatagaaagaatcactacaaggagcgcgaaatga

SEQ ID NO: 27 - DNA, 31 bp, Artificial Sequence
  Description of Artificial Sequence Synthetic primer
atgaataaggtcataaccgatttagacaaag

SEQ ID NO: 28 - DNA, 30 bp, Artificial Sequence
  Description of Artificial Sequence Synthetic primer
ttatttcgtgctccttgtggtgattttttc

SEQ ID NO: 29 - DNA, 624 bp, Helicobacter pylori
atgagagaggctatcattaaaagagcggcaaaggaattaaaagagggcatgtatgtgaatttagggataggtttgcccacgctggtggctaatgaagtgagcgggatgaatatcgttttccaaagcgagaacgggttattagggattggcgcttaccctttagaagggggcgttgatgcggatctcattaatgcaggaaaggaaaccataaccgtggtgccgggcgcttcgttttttaatagcgcggattcgtttgcgatgattcgtggggggcatattgatttagcgattttaggagggatggaagtctcacaaaatggggatttggctaattggatgatccctaaaaagctcataaaaggcatgggaggggctatggatttggtgcatggcgctaaaaaagtgattgtcatcatggagcattgcaacaaatacggggagtctaaagtgaaaaaagaatgctcattgcccttaacgggaaaaggcgtggtgcatcaattgataacggatttagcggtgtttgaattttccaataacgccatgaaattagtggaattgcaagagggggtcagccttgatcaagtgagagaaaaaacagaagccgaatttgaagtgcacctatag

SEQ ID NO: 30 - DNA, 28 bp, Artificial Sequence
  Description of Artificial Sequence Synthetic primer
atgagagaggctatcattaaaagagcgg

SEQ ID NO: 31 - DNA, 27 bp, Artificial Sequence
  Description of Artificial Sequence Synthetic primer
ttataagcgcacctcaaattcagcttc

SEQ ID NO: 32 - DNA, 717 bp, Bacillus subtilis
atgggaaaagtgctgtcatcaagcaaggaagctgcgaaactgattcatgatggggatacgctgatcgcgggagggtttgggctgtgcggcatccctgaacagctcattttgtctataagagatcagggagtaaaggatttaaccgttgtcagcaataactgcggagtcgatgactgggggcttggtttgcttctggctaacaagcaaatcaagaaaatgatcgcttcctatgtcggtgaaaataaaatttttgagcggcagtttttaagcggagagcttgaggtagagcttgttccccaaggaacgctcgctgagagaattcgtgcaggcggtgcaggcataccgggattttatacggcgacaggcgtcggcacctccatagccgagggaaaagaacataaaacattcggcggccggacttatgtgctggagcgaggcattaccggcgatgtggcgatcgtcaaagcgtggaaagcggacaccatgggcaatttgatttttaggaaaacggcgagaaatttcaatcccattgccgccatggcaggcaagatcacgattgccgaggcggaagaaatcgtggaagcaggagagctcgatccagatcacatccatacgccgggaatttacgtacagcatgtcgtgcttggcgcgagccaagaaaaacggattgaaaaacgaacagttcagcaagcatcgggaaagggtgaggccaagtga

SEQ ID NO: 33 - DNA, 24 bp, Artificial Sequence
  Description of Artificial Sequence Synthetic primer
atgggaaaagtgctgtcatcaagc

SEQ ID NO: 34 - DNA, 23 bp, Artificial Sequence
  Description of Artificial Sequence Synthetic primer
ttacttggcctcaccctttcccg

SEQ ID NO: 35 - DNA, 651 bp, Bacillus subtilis
gtgaaggaagcgagaaaacgaatggtcaaacgggctgtacaagaaatcaaggacggcatgaatgtgaatctcgggattggaatgccgacgcttgtcgcaaatgagatacccgatggcgttcacgtcatgcttcagtcggaaaacggcttgctcggaattggcccctatcctctggaaggaacggaagacgcggatttgatcaatgcgggaaaggaaacgatcactgaagtgacaggcgcctcttattttgacagcgctgagtcattcgcgatgataagaggcgggcatatcgatttagctattctcggcggaatggaggtttcggagcagggggatttggccaattggatgatcccgggcaaaatggtaaaagggatgggcggcgccatggatctcgtcaacggggcgaaacgaatcgttgtcatcatggagcacgtcaataagcatggtgaatcaaaggtgaaaaaaacatgctcccttccgctgacaggccagaaagtcgtacacaggctgattacggatttggctgtatttgattttgtgaacggccgcatgacactgacggagcttcaggatggtgtcacaattgaagaggtttatgaaaaaacagaagctgatttcgctgtaagccagtctgtactcaattcttaa

SEQ ID NO: 36 - DNA, 24 bp, Artificial Sequence
  Description of Artificial Sequence Synthetic primer
tgaaggaagcgagaaaacgaatgg

SEQ ID NO: 37 - DNA, 30 bp, Artificial Sequence
  Description of Artificial Sequence Synthetic primer
ttaagaattgagtacagactggcttacagc

SEQ ID NO: 38 - DNA, 651 bp, Escherichia coli
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 39 - DNA, 663 bp, Escherichia coli
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 40 - DNA, 657 bp, Clostridium acetobutylicum
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 41 - DNA, 38 bp, Artificial Sequence
  Description of Artificial Sequence Synthetic primer
atgaactctaaaataattagatttgaaaatttaaggtc

SEQ ID NO: 42 - DNA, 36 bp, Artificial Sequence
  Description of Artificial Sequence Synthetic primer
ttatgcaggctcctttactatataatttataagaac

SEQ ID NO: 43 - DNA, 666 bp, Clostridium acetobutylicum
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 44 - DNA, 37 bp, Artificial Sequence
  Description of Artificial Sequence Synthetic primer
atgattaatgataaaaacctagcgaaagaaataatag

SEQ ID NO: 45 - DNA, 28 bp, Artificial Sequence
  Description of Artificial Sequence Synthetic primer
ttaaacagccatgggtctaagttcattg

SEQ ID NO: 46 - DNA, 735 bp, Clostridium acetobutylicum
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 47 - DNA, 35 bp, Artificial Sequence
  Description of Artificial Sequence Synthetic primer
atgttaaaggatgaagtaattaaacaaattagcac

SEQ ID NO: 48 - DNA, 37 bp, Artificial Sequence
  Description of Artificial Sequence Synthetic primer
ttacttaagataatcatatataacttcagctctaggc

SEQ ID NO: 49 - DNA, 741 bp, Clostridium beijerinckii
atgttagaaagtgaagtatctaaacaaattacaactccacttgctgctccagcgtttcctagaggaccatataggtttcacaatagagaatatctaaacattatttatcgaactgatttagatgctcttcgaaaaatagtaccagagccacttgaattagatagagcatatgttagatttgaaatgatggctatgcctgatacaaccggactaggctcatatacagaatgtggtcaagctattccagtaaaatataatggtgttaagggtgactacttgcatatgatgtatctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 50 - DNA, 37 bp, Artificial Sequence
  Description of Artificial Sequence Synthetic primer
atgttagaaagtgaagtatctaaacaaattacaactc

SEQ ID NO: 51 - DNA, 36 bp, Artificial Sequence
  Description of Artificial Sequence Synthetic primer
ttattttactgaaagataatcatgtacaaccttagg

SEQ ID NO: 52 - DNA, 990 bp, Escherichia coli
atgcatattacatacgatctgccggttgctattgatgacattattgaagcgaaacaacgactggctgggcgaatttataaaacaggcatgcctcgctccaactattttagtgaacgttgcaaaggtgaaatattcctgaagtttgaaaatatgcagcgtacgggttcatttaaaattcgtggcgcatttaataaattaagttcactgaccgatgcggaaaaacgcaaaggcgtggtggcctgttctgcgggcaaccatgcgcaaggggtttccctctcctgcgcgatgctgggtatcgacggtaaagtggtgatgccaaaaggtgcgccaaaatccaaagtagcggcaacgtgcgactactccgcagaagtcgttctgcatggtgataacttcaacgacactatcgctaaagtgagcgaaattgtcgaaatggaaggccgtatttttatcccaccttacgatgatccgaaagtgattgctggccagggaacgattggtctggaaattatggaagatctctatgatgtcgataacgtgattgtgccaattggtggtggcggtttaattgctggtattgcggtggcaattaaatctattaacccgaccattcgtgttattggcgtacagtctgaaaacgttcacggcatggcggcttctttccactccggagaaataaccacgcaccgaactaccggcaccctggcggatggttgtgatgtctcccgcccgggtaatttaacttacgaaatcgttcgtgaattagtcgatgacatcgtgctggtcagcgaagacgaaatcagaaacagtatgattgccttaattcagcgcaataaagtcgtcaccgaaggcgcaggcgctctggcatgtgctgcattattaagcggtaaattagaccaatatattcaaaacagaaaaaccgtcagtattatttccggcggcaatatcgatctttctcgcgtctctcaaatcaccggtttcgttgacgcttaa

SEQ ID NO: 53 - DNA, 25 bp, Artificial Sequence
  Description of Artificial Sequence Synthetic primer
atgcatattacatacgatctgccgg

SEQ ID NO: 54 - DNA, 22 bp, Artificial Sequence
  Description of Artificial Sequence Synthetic primer
ttaagcgtcaacgaaaccggtg

SEQ ID NO: 55 - DNA, 1024 bp, Escherichia coli
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtaggcgtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttgg

SEQ ID NO: 56 - DNA, 21 bp, Artificial Sequence
  Description of Artificial Sequence Synthetic primer
atggctgactcgcaacccctg

SEQ ID NO: 57 - DNA, 25 bp, Artificial Sequence
  Description of Artificial Sequence Synthetic primer
ttaacccgccaaaaagaacctgaac

SEQ ID NO: 58 - DNA, 1024 bp, Escherichia coli
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaac

SEQ ID NO: 59 - DNA, 25 bp, Artificial Sequence
  Description of Artificial Sequence Synthetic primer
atgaacgaacaatattccgcattgc

SEQ ID NO: 60 - DNA, 24 bp, Artificial Sequence
  Description of Artificial Sequence Synthetic primer
ttagccggtattacgcatacctgc

SEQ ID NO: 61 - DNA, 1024 bp, Streptococcus mutans
atggcaactgtcaaaactaacactgacgtttttgaaaaagcctgggaaggctttaaaggaactgactggaaagacagagcaagcatttctcgctttgttcaagacaactacactccatatgacggagacgaaagttttcttgccggccctactgaacgttcacttcacatcaaaaaagtcgtagaagaaactaaagcgcattacgaagaaacacgttttccaatggatacacgtattacatctattgctgatatcccagcaggttatattgacaaggaaaatgaattgatttttggtatccaaaacgatgaactttttaagctgaacttcatgccaaaaggcggtattcgcatggctgaaacagctttgaaagaacatggttatgaaccagaccctgccgttcatgaaatctttaccaaatatgcaacaaccgttaatgatggtatctttcgtgcttacacttcaaacattcgccgtgcacgtcatgcccacactgtaactggtctcccagatgcatactctcgcggacgtattattggagtttatgcccgtcttgctctctatggtgctgactacttgatgcaagaaaaagtgaacgactggaactcaattgctgaaattgatgaagaatcaattcgtcttcgtgaagaaatcaatcttcaatatcaggcacttggcgaagtagtgcggttgggtgatctgtatggtcttgatgttcgcaaacctgctatgaatgttaaagaagctatccaatggattaatatcgcctttatggctgtctgccgcgttatcaatggtgctgcaacttctcttggacgtgtcccaatcgttcttgatatctttgcagaacgtgaccttgctcgtggcactttcactgaatcagaaatccaagaattcgttgatgacttcgttatgaaacttcgtacggttaaatttgcacgtactaaggcttatgacgaactttactcaggtgacccaacatttattacgacttctatggctggtatgggagctgatggacgtc

SEQ ID NO: 62 - DNA, 28 bp, Artificial Sequence
  Description of Artificial Sequence Synthetic primer
atggcaactgtcaaaactaacactgacg

SEQ ID NO: 63 - DNA, 30 bp, Artificial Sequence
  Description of Artificial Sequence Synthetic primer
ttatttgttgttaaccaagtctgtagctgc

SEQ ID NO: 64 - DNA, 792 bp, Streptococcus mutans
atgatagaaaaagttgactacgaaaaagtaacaggacttgttaattctacagaatcttttgggtctgtagacggacctggtatacgctttgttgtttttatgcaagggtgccaaatgcgttgtcaatattgccacaatcctgatacttgggcaatgaagaatgatagagcaacagaaaggactgcaggagatgtctttaaagaagctttacgttttaaagatttttggggagatacaggaggtattactgtttctggtggtgaagcaacgctccagatggattttttaattgccctcttttctttagcaaaagaaaagggaattcatacgaccttggatacctgtgctctgacttttagaaacacaccaaaatatcttgaaaaatatgaaaagttaatggctgtcactgatttagtattgttagatattaaagagattaatcctgaccaacataaaattgtcactggtcatagcaataaaactattttagcttgtgcgcgttatttatctgatattggaaaacctgtttggattcgccatgtcttagtccctggtctgactgatcgggatgaagacttaataaagttgggtgagtatgtcaaaacactgaagaatgttcaacggtttgaaattcttccttatcatacaatgggtgaattcaaatggcgtgaattagggattccttatcctttggaaggtgttaaaccgccaacaccagatcgtgtgcgcaatgctaaaaagttaatgcatacggaaacatatgaagaatataaaaagaggattaatcattaa

SEQ ID NO: 65 - DNA, 35 bp, Artificial Sequence
  Description of Artificial Sequence Synthetic primer
atgatagaaaaagttgactacgaaaaagtaacagg

SEQ ID NO: 66 - DNA, 40 bp, Artificial Sequence
  Description of Artificial Sequence Synthetic primer
ttaatgattaatcctctttttatattcttcatatgtttcc

SEQ ID NO: 67 - DNA, 1024 bp, Haemophilus influenzae
atgactatgtcagaacttaatgaaatgcaaaaattggcgtgggctggttttgctggtggcgattggcaagaaaatgtcaatgtacgtgactttatccaaaaaaactataccccttatgaaggcgatgactctttcttagcaggtccaaccgaagcaacaaccaagctttgggaatctgtgatggaaggtattaaaattgaaaaccgtactcacgcgccattagattttgatgaacatacaccatctaccattatctctcacgcacctggttacattaacaaagatttagaaaaaatcgttggtcttcaaactgatgaacctttaaaacgtgccattatgccattcggtggtatcaaaatggtggaaggttcttgtaaagtttatggtcgtgaacttgatccaaaagtgaaaaaaatcttcactgaataccgtaaaacacataaccaaggtgtattcgatgtttacacgccagatattttacgttgccgtaaatctggggtattaactggtcttccagatgcttatggtcgtggtcgtatcatcggtgactaccgtcgtgtagcactttatggtgtagatttcttaatgaaagataaatacgcacaattctcttctttacaaaaagatttagaagatggcgtaaatcttgaagcaacaattcgtttacgtgaagaaatcgcagaacaacaccgtgcattaggtcaattaaaacaaatggcagcaagctatggttatgatatttctaacccagcaactaatgctcaagaagccattcaatggatgtactttgcttatcttgctgcaataaaatcacaaaatggtgctgcaatgtcattcggtcgtaccgcaacctttattgacgtgtacatcgaacgtgatttaaaagcaggaaaaattactgaaactgaagcgcaagaattagttgaccacttagttatgaaacttcgtatggttcgtttcttacgtacacctgaatacgatcaattattctctggtgacccaatgtgggcaactgaaaccatcg

SEQ ID NO: 68 - DNA, 36 bp, Artificial Sequence
  Description of Artificial Sequence Synthetic primer
atgactatgtcagaacttaatgaaatgcaaaaattg

SEQ ID NO: 69 - DNA, 34 bp, Artificial Sequence
  Description of Artificial Sequence Synthetic primer
ttacattgactctgtgaaagttctagtaattacg

SEQ ID NO: 70 - DNA, 741 bp, Haemophilus influenzae
atgtcagttcttggacgaattcactcttttgaatcctgtggcactgtagatgggccaggtattcgttttattttatttatgcaaggctgcttgatgcgctgcaaatattgccacaatcgtgatacttgggatcttgaaggtggtaaagaaatcagtgtcgaagatttaatgaaagaagtcgtgacttatcgccattttatgaatgctactggcggtggtgtcacagcatctggtggcgaggctgtgttacaagcagagtttgtacgcgattggttccgtgcttgtaaagaggaagggattaatacttgcttagatacaaatggttttgtacgtcattatgatcatattattgatgaattattagatgtaacagatcttgttttacttgatttaaaagaacttaatgatcaagttcatcaaaatcttattggggtgccaaataaacgtacccttgaatttgcaaaatatttgcaaaaacgtaatcaacatacctggattcgttatgttgtggttcctggttatactgatagcgatcacgatgtgcatttattaggtcagtttattgaaggtatgaccaatattgaaaaagttgaacttcttccttatcatcgattaggtgtgcataaatggaaaacccttgggttagattatgagcttgaaaatgtattaccgccaactaaagaatccttagaacatattaaaacaatcctagaaggttatggacacactgtaaaattctag

SEQ ID NO: 71 - DNA, 31 bp, Artificial Sequence
  Description of Artificial Sequence Synthetic primer
atgtcagttcttggacgaattcactcttttg

SEQ ID NO: 72 - DNA, 34 bp, Artificial Sequence
  Description of Artificial Sequence Synthetic primer
ctagaattttacagtgtgtccataaccttctagg

(19) United States
(12) Patent Application Publication
(10) Pub. No.: US 2010/0184173 A1
(43) Pub. Date: Jul. 22, 2010

(54) MICROORGANISMS FOR THE PRODUCTION OF METHYL ETHYL KETONE AND 2-BUTANOL

(75) Inventors: Mark J. Burk, San Diego, CA (US); Priti Pharkya, San Diego, CA (US); Anthony P. Burgard, Bellefonte, PA (US)

Correspondence Address: MCDERMOTT, WILL & EMERY, 11682 EL CAMINO REAL, SUITE 400, SAN DIEGO, CA, 92130-2047, US

(73) Assignee: Genomatica, Inc., San Diego, CA (US)

(21) Appl. No.: 12/618,545

(22) Filed: Nov. 13, 2009

Related U.S. Application Data
(60) Provisional application No. 61/114,977, filed on Nov. 14, 2008.
(60) Provisional application No. 61/155,114, filed on Feb. 24, 2009.
(60) Provisional application No. 61/185,967, filed on Jun. 10, 2009.

(51) Int. Cl. C12P 7/26; C12N 1/00; C12P 7/16

(52) U.S. Cl. 435/148; 435/243; 435/160

(57) ABSTRACT

A non-naturally occurring microbial organism having a methyl ethyl ketone pathway includes at least one exogenous nucleic acid encoding a methyl ethyl ketone pathway enzyme expressed in a sufficient amount to produce methyl ethyl ketone. The methyl ethyl ketone pathway includes a β-ketothiolase, a β-ketovalerate decarboxylase and an enzyme selected from the group consisting of a β-ketovaleryl-CoA hydrolase and a β-ketovaleryl-CoA transferase. Alternatively, the methyl ethyl ketone pathway includes a 2-methylacetoacetyl-CoA thiolase, a 2-methylacetoacetate decarboxylase and an enzyme selected from the group consisting of a 2-methylacetoacetyl-CoA hydrolase and a 2-methylacetoacetyl-CoA transferase. Either pathway can further include a methyl ethyl ketone reductase to produce 2-BuOH. A method for producing methyl ethyl ketone or 2-BuOH includes culturing these non-naturally occurring microbial organisms under conditions, and for a sufficient period of time, to produce methyl ethyl ketone or 2-BuOH.

### RELATED APPLICATIONS

This application claims the benefit of priority of U.S. Provisional Application Ser. No. 61/114,977, filed Nov. 14, 2008; U.S. Provisional Application Ser. No. 61/155,114, filed Feb. 24, 2009; and U.S. Provisional Application Ser. No. 61,185,967, filed Jun. 10, 2009, each of which the entire contents are incorporated herein by reference.

### BACKGROUND OF THE INVENTION

This invention relates generally to the production of commodity and specialty chemicals and, more specifically to an integrated bioprocess for producing methyl ethyl ketone and 2-butanol.

Methyl ethyl ketone (MEK) is a four carbon ketone that is currently manufactured either through hydration of butylene followed by oxidation (e.g., ExxonMobile), or from benzene as by-product of phenol production (e.g., Shell process). MEK is mainly used as a large volume solvent for coatings, adhesives, and inks, as well as a chemical intermediate. 2-butanol, like MEK, is used as a solvent and is employed in industrial cleaners and paint removers. Some volatile esters of 2-butanol have pleasant aromas and are used in perfumes and artificial flavors.

MEK has a global market of approximately 2.3 B lb per year with an annual growth rate of 4-4.5%. Demand for MEK in general is expected to significantly increase due to its recent delisting from the EPAs hazardous air pollutants classification. Demand for MEK in China is expected to continue increasing at the rate of 8-9% per year. Rising butylene and benzene prices are threatening the modest margins of the petrochemical processes and new process technologies are being sought.

Thus, there exists a need for compositions and methods that reduce the use of petroleum-based synthesis of MEK, as well as 2-butanol. The present invention satisfies this need and provides related advantages as well.

### SUMMARY OF THE INVENTION

In some aspects, embodiments disclosed herein relate to a non-naturally occurring microbial organism having a methyl ethyl ketone pathway that includes at least one exogenous nucleic acid encoding a methyl ethyl ketone pathway enzyme expressed in a sufficient amount to produce methyl ethyl ketone. The methyl ethyl ketone pathway includes a β-ketothiolase, a β-ketovalerate decarboxylase and an enzyme selected from the group consisting of a β-ketovaleryl-CoA hydrolase and a β-ketovaleryl-CoA transferase.

In some aspects, embodiments disclosed herein relate to a non-naturally occurring microbial organism having a methyl ethyl ketone pathway that includes at least one exogenous nucleic acid encoding a methyl ethyl ketone pathway enzyme expressed in a sufficient amount to produce methyl ethyl ketone. The methyl ethyl ketone pathway includes a 2-methylacetoacetyl-CoA thiolase, a 2-methylacetoacetate decarboxylase and an enzyme selected from the group consisting of a 2-methylacetoacetyl-CoA hydrolase and a 2-methylacetoacetyl-CoA transferase.

In some aspects, embodiments disclosed herein relate to a non-naturally occurring microbial organism having a 2-BuOH pathway that includes either of the two aforementioned methyl ethyl ketone pathways and further including a methyl ethyl ketone reductase to produce 2-BuOH.

In some aspects, embodiments disclose herein relate to a method for producing methyl ethyl ketone or 2-BuOH that includes culturing these non-naturally occurring microbial organisms under conditions, and for a sufficient period of time, to produce methyl ethyl ketone or 2-BuOH.

### BRIEF DESCRIPTION OF THE DRAWINGS

FIG. 1 shows the metabolic pathway for methyl ethyl ketone production via β-ketovaleryl-CoA intermediate. Abbreviations: GLC—glucose, PEP—phosphoenolpyruvate, PYR—pyruvate, FOR—formate, ACCOA—acetyl-CoA, OAA, oxaloacetate, MAL—malate, FUM—fumarate, SUCC—succinate, SUCCOA—succinyl-CoA, (R)-MMCOA—R-methylmalonyl-CoA, (S)-MMCOA—(S)-methylmalonyl-CoA, PPCOA—propionyl-CoA, BKVCOA—β-ketovaleryl-CoA, BKV—β-ketovalerate, MEK—methyl ethyl ketone.

FIG. 2 shows the metabolic pathway for methyl ethyl ketone production via a 2-methylacetoacetyl-CoA intermediate. Abbreviations: GLC—glucose, PEP—phosphoenolpyruvate, PYR—pyruvate, FOR—formate, ACCOA—acetyl-CoA, OAA, oxaloacetate, MAL—malate, FUM—fumarate, SUCC—succinate, SUCCOA—succinyl-CoA, (R)-MMCOA—R-methylmalonyl-CoA, (S)-MMCOA—(S)-methylmalonyl-CoA, PPCOA—propionyl-CoA, 2MAACOA—2-methylacetoacetyl-CoA, 2MAA—2-methylacetoacetate, MEK—methyl ethyl ketone.

FIG. 3 shows the metabolic pathway for 2-butanol production via a β-ketovaleryl-CoA intermediate. Abbreviations: GLC—glucose, PEP—phosphoenolpyruvate, PYR—pyruvate, FOR—formate, ACCOA—acetyl-CoA, OAA, oxaloacetate, MAL—malate, FUM—fumarate, SUCC—succinate, SUCCOA—succinyl-CoA, (R)-MMCOA—R-methylmalonyl-CoA, (S)-MMCOA—(S)-methylmalonyl-CoA, PPCOA—propionyl-CoA, BKVCOA—β-ketovaleryl-CoA, BKV—β-ketovalerate, MEK—methyl ethyl ketone, 2BuOH—2-butanol.

FIG. 4 shows the metabolic pathway for 2-butanol production via a 2-methylacetoacetyl-CoA intermediate. Abbreviations: GLC—glucose, PEP—phosphoenolpyruvate, PYR—pyruvate, FOR—formate, ACCOA—acetyl-CoA, OAA, oxaloacetate, MAL—malate, FUM—fumarate, SUCC—succinate, SUCCOA—succinyl-CoA, (R)-MMCOA—R-methylmalonyl-CoA, (S)-MMCOA—(S)-methylmalonyl-CoA, PPCOA—propionyl-CoA, 2MAACOA—2-methylacetoacetyl-CoA, 2MAA—2-methylacetoacetate, MEK—methyl ethyl ketone, 2BuOH—2-butanol.

FIG. 5 shows an exemplary metabolic pathway for methyl ethyl ketone production via a β-ketovaleryl-CoA intermediate incorporating an alternate pathway to propionyl-CoA via threonine.

FIG. 6 shows growth of E. coli and S. cerevisiae in medium containing various concentrations of MEK.

### DETAILED DESCRIPTION OF THE INVENTION

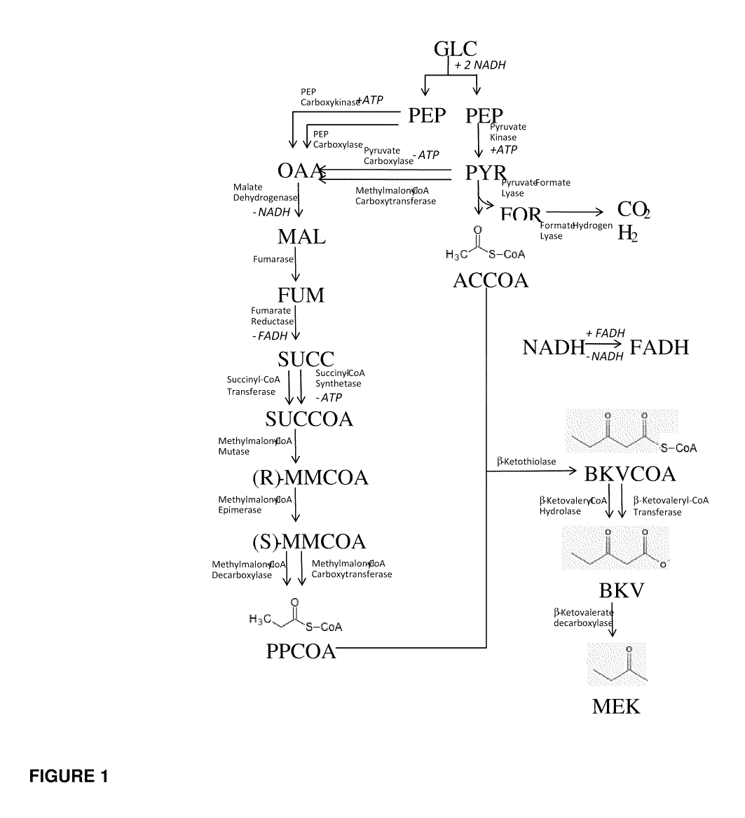

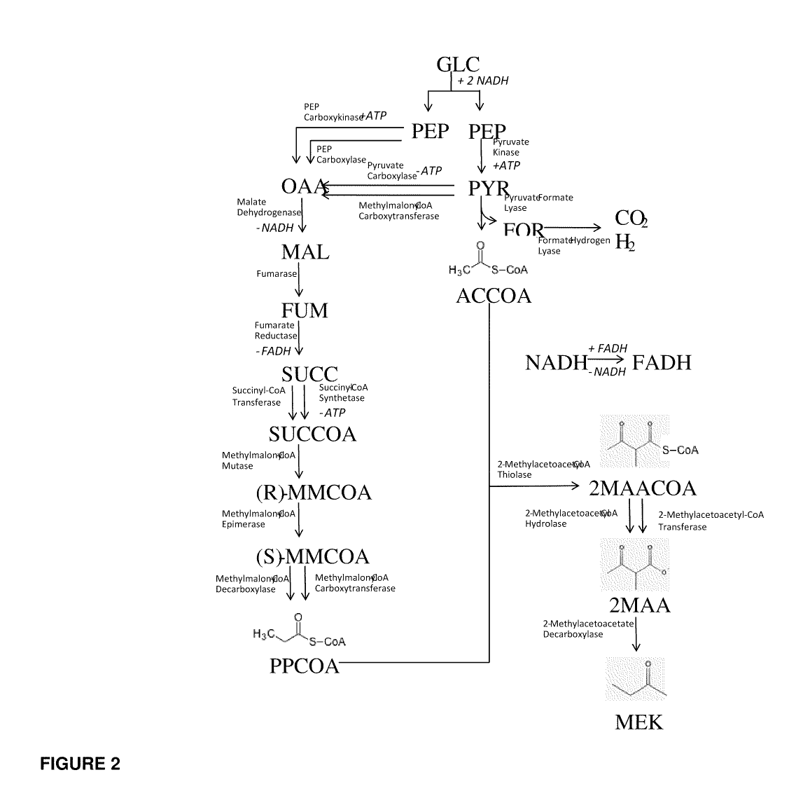

Embodiments of the present invention provide non-naturally occurring microbial organisms having redox-balanced anaerobic pathways to MEK that proceed from one phosphoenolpyruvate (PEP) molecule and one pyruvate molecule as exemplified in FIGS. 1 and 2. Both PEP and pyruvate are produced in high quantities via glycolysis. PEP and pyruvate can be converted to propionyl-CoA and acetyl-CoA, respectively, by several common metabolic reactions in both pathways. As shown in FIG. 1, PEP can be converted to oxaloacetate by means of PEP carboxykinase or PEP carboxylase. Alternatively, PEP can be converted first to pyruvate by pyruvate kinase and then to oxaloacetate by methylmalonyl-CoA carboxytransferase. Oxaloacetate can be converted to propionyl-CoA by means of the reductive TCA cycle, a methylmutase, a decarboxylase, an epimerase and carboxytransferase. Pyruvate can be converted to acetyl-CoA by means of pyruvate formate lyase resulting in the co-generation of one mol of formate per mol of MEK produced. The pathways disclosed herein can provide a theoretical yield of one mol of MEK per mol of glucose metabolized. They can also generate 2 moles of ATP per mole of glucose metabolized assuming the theoretical maximum yield of MEK.

One exemplary organism that can be used in the production of methyl ethyl ketone is Saccharomyces cerevisiae. S. cerevisiae, a natural ethanol producer that is widely employed industrially, can be modified to produce MEK instead of ethanol. Both ethanol and MEK can be purified from a fermentation broth via similar distillation-based strategies given their similar boiling points [i.e., ethanol (78° C.), MEK (bp=80° C.)]. Thus S. cerevisiae strains engineered for MEK production can potentially replace the ethanol-producing strains employed in existing fermentation facilities leading to the generation of a higher value chemical. MEK production by means of the pathways disclosed herein can be done anaerobically in the existing ethanol fermentation vessels with little or no equipment modification.

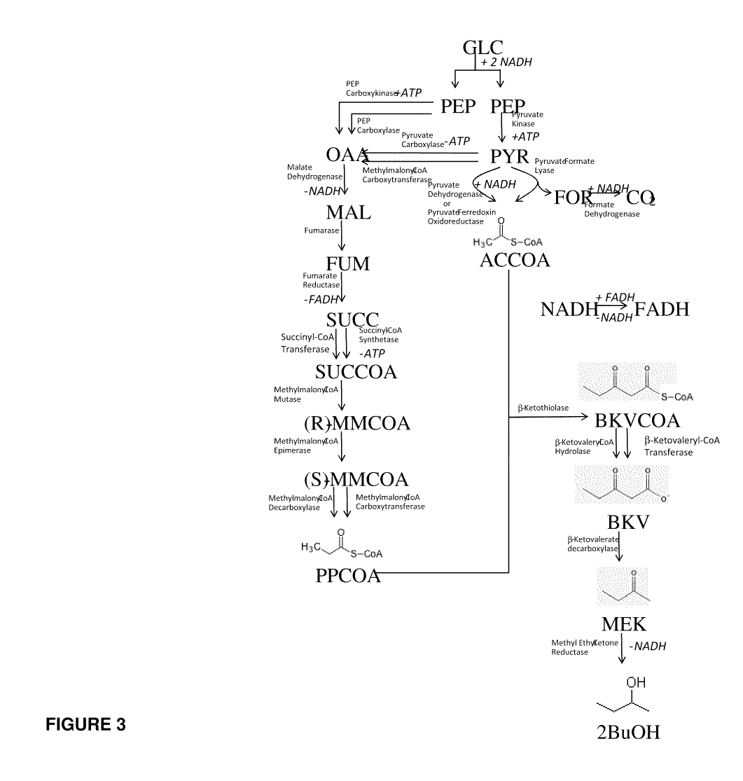

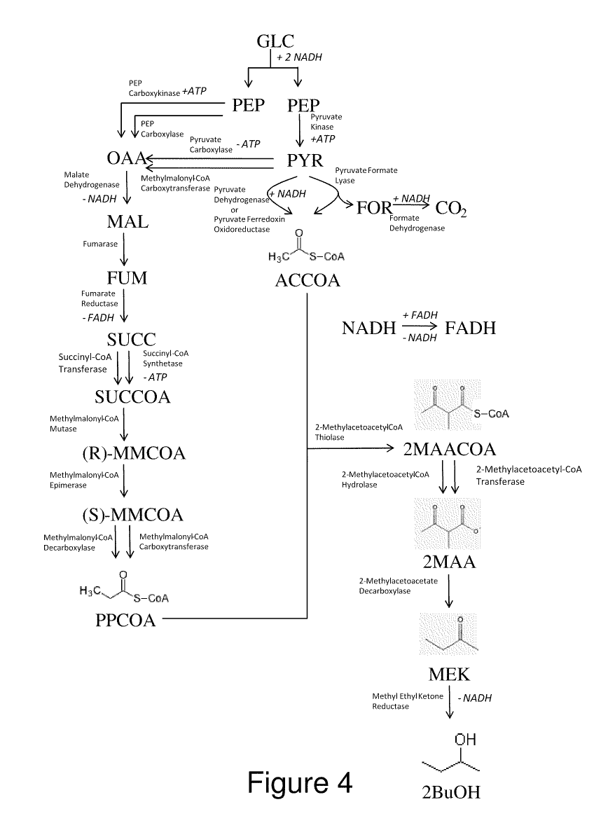

Embodiments of the present invention also provide non-naturally occurring microbial organisms that can form 2-butanol from renewable resources as shown in FIGS. 3 and 4. Specifically the organism includes all enzymes utilized in the production of MEK from acetyl-CoA and propionyl-CoA with the exception of formate hydrogen lyase. Instead, formate can be converted to carbon dioxide by a formate dehydrogenase that provides an additional reducing equivalent that can be used for 2-butanol synthesis from MEK. Alternatively, this reducing equivalent can be obtained by pyruvate dehydrogenase or pyruvate ferredoxin oxidoreductase.

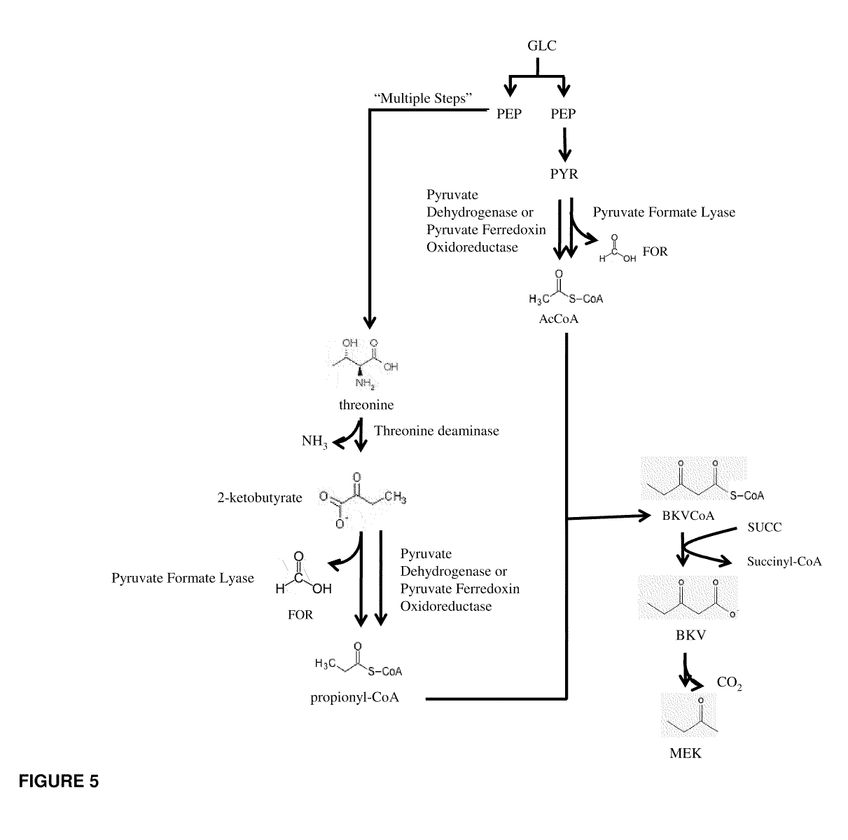

Embodiments of the present invention also provide non-naturally occurring microbial organisms that can form MEK or 2-butanol via any of the pathways shown in FIGS. 1-4, exchanging the oxaloacetate pathway to propionyl-CoA with an alternate pathway via threonine as exemplified in FIG. 5. This alternate pathway can replace or supplement the oxaloacetate to propionyl-CoA pathway in each of FIGS. 1-4, with FIG. 5 being merely exemplary. In FIG. 5, an MEK pathway is shown in which propionyl-CoA is generated from threonine via a threonine deaminase, followed by conversion to propionyl-CoA by action of a pyruvate formate lyase and a pyruvate formate lyase activating enzyme. Alternatively, 2-ketobutyrate can be converted to propionyl-CoA by pyruvate dehydrogenase or pyruvate ferredoxin oxidoreductase. While FIG. 5 shows MEK production by way of a β-ketovaleryl-CoA intermediate, it will be understood that the alternate condensation to 2-methylacetoacetyl-CoA can be used. Furthermore, one skilled in the art will appreciate that MEK produced via the pathways shown in FIG. 5 can be further converted to 2-butanol by further pathways disclosed herein. Threonine can be generated from aspartate, which in turn feeds from the TCA cycle by way of oxaloacetate. The threonine pathway contains no vitamin B12-dependant enzymes. Thus, the threonine pathway can be beneficial for organisms that cannot take up vitamin B 12 or cannot be engineered to take up B 12.

Engineering these pathways into a microorganism such as S. cerevisiae, for example, involves cloning an appropriate set of genes encoding a set of enzymes into a production host, optimizing fermentation conditions, and assaying product formation following fermentation. To engineer a production host for the production of MEK or 2-butanol, one or more exogenous DNA sequence(s) can be expressed in a microorganism. In addition, the microorganism can have endogenous gene(s) functionally disrupted, deleted or overexpressed. The metabolic modifications disclosed herein enable the production of MEK or 2-butanol using renewable feedstock.

In some embodiments, the invention provides non-naturally occurring microbial organisms that include at least one exogenous nucleic acid that encode a methyl ethyl ketone pathway enzyme expressed in a sufficient amount to produce methyl ethyl ketone.

In another embodiment, the invention provides non-naturally occurring microbial organisms that include at least one exogenous nucleic acid that encode a 2-butanol pathway enzyme expressed in a sufficient amount to produce 2-butanol.

In still other embodiments, the invention provides methods for producing methyl ethyl ketone and 2-butanol. Such methods involve culturing the microbial organisms described herein.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a methyl ethyl ketone and/or 2-butanol biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

The non-naturally occurring microbal organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as S. cerevisiae and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the S. cerevisiae metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having methyl ethyl ketone and/or 2-butanol biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, the present invention provides a non-naturally occurring microbial organism that includes a microbial organism having a methyl ethyl ketone biosynthetic pathway. The non-naturally occurring microbial organism includes at least one exogenous nucleic acid encoding a methyl ethyl ketone pathway enzyme expressed in a sufficient amount to produce methyl ethyl ketone. In some embodiments, a methyl ethyl ketone pathway includes a β-ketothiolase, β-ketovalerate decarboxylase and an enzyme such as a β-ketovaleryl-CoA hydrolase, or a β-ketovaleryl-CoA transferase.

The chemical transformations involved in the production of MEK from propionyl-CoA and acetyl-CoA by the pathway, exemplified in FIG. 1, are analogous to those of acetone production from two acetyl-CoA molecules. Acetone was recently produced as part of the isopropanol production pathway in recombinant E. coli. Specifically, isopropanol production was achieved in recombinant E. coli following expression of two heterologous genes from C. acetobutylicum (thl and adc encoding acetoacetyl-CoA thiolase and acetoacetate decarboxylase, respectively) and one from C. beijerinckii (adh encoding a secondary alcohol dehydrogenase), along with the increased expression of the native atoA and atoD genes which encode acetoacetyl-CoA:acetyl-CoA transferase activity. (Hanai et al., Appl. Environ. Microbiol. 73:7814-7818 (2007), Also see Jojima et al., Appl. Microbiol. Biotechnol. 77: 1219-1224 (2008).) Acetone production required all but the expression of the secondary alcohol dehydrogenase.

The first step in the net conversion of propionyl-CoA and acetyl-CoA to MEK involves their condensation to form 3-oxopentanoyl-CoA or, equivalently, β-ketovaleryl-CoA. The gene products of bktB and bktC from Ralstonia eutropha (formerly known as Alcaligenes eutrophus) exhibit this activity. (Slater et al., J. Bacteriol. 180:1979-1987 (1998).) The sequence of the BktB protein can be accessed by the following GenBank accession number, as shown in Table 1 below, while the sequence of the BktC protein has not been reported. Further, it was reported (Aldor and Keasling, Biotechnol Bioeng. 76:108-114 (2001); Aldor et al., Appl Environ. Microbiol 68:3848-3854 (2002)) that the phaA gene from Acinetobacter sp. catalyzes the formation of β-ketovaleryl-CoA from propionyl-CoA and acetyl-CoA.

TABLE 1

| | | |
|---|---|---|
| bktB | YP_725948.1 | Ralstonia eutropha |
| phaA | AAA99475 | Acinetobacter sp. strain RA3849 |

These sequences and sequences for subsequent enzymes identified herein can be used to identify homologous proteins in GenBank or other databases through sequence similarity searches (e.g. BLASTp). The resulting homologous proteins and their corresponding gene sequences provide additional DNA sequences for transformation into S. cerevisiae or other microbial organisms.

For example, Gruys et al., U.S. Pat. No. 5,958,745, filed Sep. 28, 1999, report that Zoogloea ramigera possesses two ketothiolases that can form β-ketovaleryl-CoA from propionyl-CoA and acetyl-CoA and R. eutropha has a β-oxidation ketothiolase that is also capable of catalyzing this transformation. The sequences of these genes or their translated proteins have not been reported, but several genes in R. eutropha, Z. ramigera, or other organisms can be identified based on sequence homology to bktB from R. eutropha. These include those shown in Table 2 below.

TABLE 2

| | | |
|---|---|---|
| phaA | YP_725941.1 | Ralstonia eutropha |
| h16_A1713 | YP_726205.1 | Ralstonia eutropha |
| pcaF | YP_728366.1 | Ralstonia eutropha |
| h16_B1369 | YP_840888.1 | Ralstonia eutropha |
| h16_A0170 | YP_724690.1 | Ralstonia eutropha |
| h16_A0462 | YP_724980.1 | Ralstonia eutropha |
| h16_A1528 | YP_726028.1 | Ralstonia eutropha |
| h16_B0381 | YP_728545.1 | Ralstonia eutropha |
| h16_B0662 | YP_728824.1 | Ralstonia eutropha |
| h16_B0759 | YP_728921.1 | Ralstonia eutropha |
| h16_B0668 | YP_728830.1 | Ralstonia eutropha |
| h16_A1720 | YP_726212.1 | Ralstonia eutropha |
| h16_A1887 | YP_726356.1 | Ralstonia eutropha |
| phbA | P07097.4 | Zoogloea ramigera |
| bktB | YP_002005382.1 | Cupriavidus taiwanensis |
| Rmet_1362 | YP_583514.1 | Ralstonia metallidurans |
| Bphy_0975 | YP_001857210.1 | Burkholderia phymatum |

Additional ketothiolases that are known to convert two molecules of acetyl-CoA into acetoacetyl-CoA. Exemplary acetoacetyl-CoA thiolase enzymes include the gene products of atoB from E. coli (Martin et al., Nat. Biotechnol 21:796-802 (2003)), thlA and thlB from C. acetobutylicum (Hanai et al., Appl Environ Microbiol 73:7814-7818 (2007)); Winzer et al., J. Mol. Microbiol Biotechnol 2:531-541 (2000)), and ERG10 from S. cerevisiae (Hiser et al., J. Biol. Chem. 269:31383-31389 (1994)) as shown in Table 3 below.

TABLE 3

| | | |
|---|---|---|
| AtoB | NP_416728 | Escherichia coli |
| ThlA | NP_349476.1 | Clostridium acetobutylicum |
| ThlB | NP_149242.1 | Clostridium acetobutylicum |
| ERG10 | NP_015297 | Saccharomyces cerevisiae |

The conversion of β-ketovaleryl-CoA to β-ketovalerate can be carried out by a β-ketovaleryl-CoA transferase which conserves the energy stored in the CoA-ester bond. In one embodiment an enzyme for this reaction step is succinyl-CoA:3-ketoacid-CoA transferase. This enzyme converts succinate to succinyl-CoA while converting a 3-ketoacyl-CoA to a 3-ketoacid. This enzyme is not only useful for converting β-ketovaleryl-CoA to β-ketovalerate, but also for catalyzing the conversion of succinate to succinyl-CoA (see FIG. 1). Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007)), and Homo sapiens (Fukao et al., Genomics 68:144-151 (2000); Tanaka et al., Mol. Hum. Reprod. 8:16-23 (2002)) are shown in Table 4 below.

TABLE 4

| | | |
|---|---|---|
| HPAG1_0676 | YP_627417 | Helicobacter pylori |
| HPAG1_0677 | YP_627418 | Helicobacter pylori |
| ScoA | NP_391778 | Bacillus subtilis |
| ScoB | NP_391777 | Bacillus subtilis |
| OXCT1 | NP_000427 | Homo sapiens |
| OXCT2 | NP_071403 | Homo sapiens |

Another β-ketovaleryl-CoA transferase that can catalyze the conversion of β-ketovaleryl-CoA to β-ketovalerate is acetoacetyl-CoA:acetyl-CoA transferase. This enzyme normally converts acetoacetyl-CoA and acetate to acetoacetate and acetyl-CoA, but can show activity on β-ketovaleryl-CoA which is only one carbon longer than acetoacetyl-CoA. Exemplary enzymes include the gene products of atoAD from E. coli (Hanai et al., Appl Environ Microbiol 73:7814-7818 (2007)), ctfAB from C. acetobutylicum (Jojima et al., Appl Microbiol Biotechnol 77:1219-1224 (2008)), and ctfAB from Clostridium saccharoperbutylacetonicum (Kosaka et al., Biosci. Biotechnol Biochem. 71:58-68 (2007)), as shown in Table 5 below.

TABLE 5

| | | |
|---|---|---|
| AtoA | NP_416726.1 | Escherichia coli |
| AtoD | NP_416725.1 | Escherichia coli |
| CtfA | NP_149326.1 | Clostridium acetobutylicum |
| CtfB | NP_149327.1 | Clostridium acetobutylicum |
| CtfA | AAP42564.1 | Clostridium saccharoperbutylacetonicum |
| CtfB | AAP42565.1 | Clostridium saccharoperbutylacetonicum |

β-ketovalereryl-CoA can be hydrolyzed to β-ketovalerate by β-ketovaleryl-CoA hydrolase. Several eukaryotic acetyl-CoA hydrolases (EC 3.1.2.1) have broad substrate specificity. The enzyme from Rattus norvegicus brain (131) can react with butyryl-CoA, hexanoyl-CoA and malonyl-CoA. Though its sequence has not been reported, the enzyme from the mitochondrion of the pea leaf showed activity on acetyl-CoA, propionyl-CoA, butyryl-CoA, palmitoyl-CoA, oleoyl-CoA, succinyl-CoA, and crotonyl-CoA (Zeiher and Randall, Plant. Physiol. 94:20-27 (1990)). Additionally, a glutaconate CoA-transferase from Acidaminococcus fermentans was transformed by site-directed mutagenesis into an acyl-CoA hydrolase with activity on glutaryl-CoA, acetyl-CoA and 3-butenoyl-CoA (Mack and Buckel, “Conversion of glutaconate CoA-transferase from Acidaminococcus fermentans into an acyl-CoA hydrolase by site-directed mutagenesis,” FEBS. Lett. 405:209-212 (1997)). This indicates that the enzymes encoding succinyl-CoA:3-ketoacid-CoA transferases and acetoacetyl-CoA:acetyl-CoA transferases can also be used for this reaction step with certain mutations to change their function. The acetyl-CoA hydrolase, ACH1, from S. cerevisiae represents another candidate hydrolase (Buu et al., J. Biol. Chem. 278:17203-17209 (2003)). The genes associated with these enzymes are shown below in Table 6.

TABLE 6

| | | |
|---|---|---|
| acot12 | NP_570103.1 | Rattus norvegicus |
| gctA | CAA57199 | Acidaminococcus fermentans |
| gctB | CAA57200 | Acidaminococcus fermentans |
| ACH1 | NP_009538 | Saccharomyces cerevisiae |

Acetoacetate decarboxylase enzymes convert acetoacetate into carbon dioxide and acetone. Exemplary acetoacetate decarboxylase enzymes are encoded by the gene products of adc from C. acetobutylicum (Petersen and Bennett, Appl Environ. Microbiol 56:3491-3498 (1990)) and adc from Clostridium saccharoperbutylacetonicum (Kosaka et al., Biosci.Biotechnol Biochem. 71:58-68 (2007)). The enzyme from C. beijerinkii can be inferred from sequence similarity. Given the structural similarity between acetoacetate and β-ketovalerate, acetoacetate decarboxylases can also catalyze the decarboxylation of β-ketovalerate. This point was demonstrated in the case of the acetoacetate decarboxylase from Bacillus polymyxa which was successfully employed in an assay to detect β-ketovalerate, or equivalently, 3-oxopentanoate (Matiasek et al., Curr. Microbiol 42:276-281 (2001)). It was also shown that decarboxylation of β-ketovalerate can occur via non-enzymatic means. The corresponding decarboxylase genes are shown below in Table 7.

TABLE 7

| | | |
|---|---|---|
| Adc | NP_149328.1 | Clostridium acetobutylicum |
| Adc | AAP42566.1 | Clostridium saccharoperbutylacetonicum |
| Adc | YP_001310906.1 | Clostridium beijerinckii |

The non-naturally occurring microbial organism of the present invention can also have a propionyl-CoA pathway that includes at least one exogenous nucleic acid encoding a propionyl-CoA pathway enzyme expressed in a sufficient amount to produce propionyl-CoA. This can be useful even if the microbial organism produces low levels of propionyl-CoA. Thus, one or more exogenous nucleic acids can be introduced to enhance propionyl-CoA flux.

In some embodiments, a propionyl-CoA pathway enzyme includes any combination of, for example, a PEP carboxylase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

Although the net conversion of phosphoenolpyruvate to oxaloacetate is redox-neutral, the mechanism of this conversion is relevant to the overall energetics of the MEK production pathway. In one embodiment, an enzyme for the conversion PEP to oxaloacetate is PEP carboxykinase which simultaneously forms an ATP while carboxylating PEP. In most organisms, however, PEP carboxykinase serves a gluconeogenic function and converts oxaloaceate to PEP at the expense of one ATP. S. cerevisiae is one such organism whose native PEP carboxykinase, PCK1, serves a gluconeogenic role (Valdes-Hevia et al., FEBS. Lett. 258:313-316 (1989)). E. coli is another such organism, as the role of PEP carboxykinase in producing oxalacetate is reported to be minor when compared to PEP carboxylase, which does not form ATP, possibly due to the higher Kₘ for bicarbonate of PEP carboxykinase (Kim et al., Appl Environ Microbiol 70:1238-1241 (2004)). Nevertheless, activity of the native E. coli PEP carboxykinase from PEP towards oxaloacetate has been recently demonstrated in ppc mutants of E. coli K-12 (Kwon et al., J. Microbiology and Biotechnology 16:1448-1452 (2006)). These strains exhibited no growth defects and had increased succinate production at high NaHCO₃ concentrations. In some organisms, particularly rumen bacteria, PEP carboxykinase is efficient in producing oxaloacetate from PEP and generating ATP. Examples of PEP carboxykinase genes that have been cloned into E. coli include those from Mannheimia succiniciproducens (Lee et al., Gene. Biotechnol. Bioprocess Eng. 7:95-99 (2002)), Anaerobiospirillum succiniciproducens (Laivenieks et al., Appl Environ Microbiol 63:2273-2280 (1997)), and Actinobacillus succinogenes (Kim et al., Appl Environ Microbiol 70:1238-1241 (2004)). The PEP carboxykinase enzyme encoded by Haemophilus influenza is also efficient at forming oxaloacetate from PEP. The protein sequences encoding the various PEP carboxykinase genes can be identified by their GenBank accession numbers as shown in Table 8 below.

TABLE 8

| Gene | GenBank ID | Organism |
|---|---|---|
| PCK1 | NP_013023 | Saccharomyces cerevisiae |
| pck | NP_417862.1 | Escherichia coli |
| pckA | YP_089485.1 | Mannheimia succiniciproducens |
| pckA | O09460.1 | Anaerobiospirillum succiniciproducens |
| pckA | Q6W6X5 | Actinobacillus succinogenes |
| pckA | P43923.1 | Haemophilus influenza |

An additional energetically efficient route to oxaloacetate from PEP uses two enzymatic activities: pyruvate kinase and methylmalonyl-CoA carboxytransferase. Pyruvate kinase catalyzes the ATP-generating conversion of PEP to pyruvate and is encoded by the PYK1 Burke et al., J. Biol. Chem. 258:2193-2201 (1983) and PYK2 (Boles et al., J. Bacteriol. 179:2987-2993 (1997)) genes in S. cerevisiae. Methylmalonyl-CoA carboxytransferase catalyzes the conversion of pyruvate to oxaloacetate. This reaction also simultaneously catalyzes the conversion of (S)-methylmalonyl-CoA to propionyl-CoA (see FIGS. 1 and 2). An exemplary methylmalonyl-CoA carboxytransferase which is comprised of 13S, 5S, and 12S subunits can be found in Propionibacterium freudenreichii (Thornton et al., J. Bacteriol. 175:5301-5308 (1993)). The various genes encoding the enzymes for these transformations are shown below in Table 9.

TABLE 9

| | | |
|---|---|---|
| PYK1 | NP_009362 | Saccharomyces cerevisiae |
| PYK2 | NP_014992 | Saccharomyces cerevisiae |
| 1.3S subunit | P02904 | Propionibacterium freudenreichii |
| 5S subunit | Q70AC7 | Propionibacterium freudenreichii |
| 12S subunit | Q8GBW6 | Propionibacterium freudenreichii |

PEP carboxylase represents an alternative enzyme for the formation of oxaloacetate from PEP. However, because the enzyme does not generate ATP upon decarboxylating oxaloacetate, its utilization decreases the maximum ATP yield of the MEK production pathway to 1 ATP per mol of MEK formed or mol of glucose metabolized. Nevertheless, the maximum theoretical MEK yield of 1 mol/mol will remain unchanged if PEP carboxylase is utilized to convert PEP to oxaloacetate. S. cerevisiae, in particular, does not naturally encode a PEP carboxylase, but exemplary organisms that possess genes that encode PEP carboxylase include E. coli (Kai et al., Arch. Biochem. Biophys. 414:170-179 (2003)), Methylobacterium extorquens AM1 (Arps, et al. J. Bacteriol. 175:3776-3783 (1993)), and Corynebacterium glutamicum (Eikmanns, et al., Mol. Gen. Genet. 218:330-339 (1989)). The corresponding genes are shown below in Table 10.

TABLE 10

| | | |
|---|---|---|
| ppc | NP_418391 | Escherichia coli |
| ppcA | AAB58883 | Methylobacterium extorquens |
| ppc | ABB53270 | Cornebacterium glutamicum |

S. cerevisiae possesses a combination of enzymes that can convert PEP to oxaloacetate with a stoichiometry identical to that of PEP carboxylase. These enzymes are encoded by pyruvate kinase, PYK1 (Burke et al., J. Biol. Chem. 258:2193-2201 (1983)) or PYK2 (Boles et al., J. Bacteriol. 179:2987-2993 (1997)), and pyruvate carboxylase, PYC1 (Walker et al., Biochem. Biophys. Res. Commun. 176:1210-1217 (1997)) or PYC2 (Walker et al., Biochem. Biophys. Res. Commun, 176:1210-1217 (1991)) as shown in Table 11 below.

TABLE 11

| | | |
|---|---|---|
| PYK1 | NP_009362 | Saccharomyces cerevisiae |
| PYK2 | NP_014992 | Saccharomyces cerevisiae |
| PYC1 | NP_011453 | Saccharomyces cerevisiae |
| PYC2 | NP_009777 | Saccharomyces cerevisiae |

Oxaloacetate can be converted to succinate by means of three enzymes in S. cerevisiae that are part of the reductive tricarboxylic acid cycle. These enzymes are malate dehydrogenase, fumarase, and fumarate reductase. S. cerevisiae possesses three copies of malate dehydrogenase, MDH1 (McAlister-Henn and Thompson, J. Bacteriol. 169:5157-5166 (1987)), MDH2 (Gibson et al., J. Biol. Chem. 278:25628-25636 (2003); Muratsubaki and Enomoto Arch. Biochem. Biophys. 352:175-181 (1998)), and MDH3 (Steffan and McAlister-Henn, J. Biol. Chem. 267:24708-24715 (1992)), which localize to the mitochondrion, cytosol, and peroxisome, respectively. S. cerevisiae contains one copy of a fumarase-encoding gene, FUM1, whose product localizes to both the cytosol and mitochondrion (Sass et al., J. Biol. Chem. 278:45109-45116 (2003)). Fumarate reductase is encoded by two soluble enzymes, FRDS1 (Enomoto et al., DNA. Res. 3:263-267 (1996)) and FRDS2 (Muratsubaki and Enomoto, Arch. Biochem. Biophys. 352:175-181 (1998)), which are required for anaerobic growth on glucose (Arikawa et al., FEMS. Microbiol Lett. 165:111-116 (1998)). The various genes outlined for the transformation of oxaloacetate to succinate are shown below in Table 12.

TABLE 12

| | | |
|---|---|---|
| MDH1 | NP_012838 | Saccharomyces cerevisiae |
| MDH2 | NP_014515 | Saccharomyces cerevisiae |
| MDH3 | NP_010205 | Saccharomyces cerevisiae |
| FUM1 | NP_015061 | Saccharomyces cerevisiae |
| FRDS1 | P32614 | Saccharomyces cerevisiae |
| FRDS2 | NP_012585 | Saccharomyces cerevisiae |

The conversion of succinate to succinyl-CoA can be carried out by a succinyl-CoA transferase that does not use energy in the form of ATP or GTP. S. cerevisiae, in particular, does not convert succinate to succinyl-CoA via a transferase, but this type of reaction is common in a number of organisms. One such enzyme that effects this transformation is succinyl-CoA:3-ketoacid-CoA transferase. This enzyme converts succinate to succinyl-CoA while converting a 3-ketoacyl-CoA to a 3-ketoacid. Thus, this enzyme is useful not only for activating succinate to succinyl-CoA, but also for converting β-ketovaleryl-CoA to β-ketovalerate in the MEK pathway (see FIGS. 1 and 2). Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007)), and Homo sapiens (Fukao et al., Genomics, 68:144-151 (2000); Tanaka et al., Mol. Hum. Reprod. 8:16-23 (2002)) as shown in Table 13 below.

TABLE 13

| | | |
|---|---|---|
| HPAG1_0676 | YP_627417 | Helicobacter pylori |
| HPAG1_0677 | YP_627418 | Helicobacter pylori |
| ScoA | NP_391778 | Bacillus subtilis |
| ScoB | NP_391777 | Bacillus subtilis |
| OXCT1 | NP_000427 | Homo sapiens |
| OXCT2 | NP_071403 | Homo sapiens |

Another exemplary succinyl-CoA transferase is the gene product of cat1 of Clostridium kluyveri that has been shown to exhibit succinyl-CoA:acetyl-CoA transferase activity (Sohling and Gottschalk, J Bacteriol. 178:871-880 (1996)). In addition, the activity is present in Trichomonas vaginalis (van Grinsven et al., J. Biol. Chem. 283:1411-1418 (2008)) and Trypanosoma brucei (Riviere et al., J. Biol. Chem. 279:45337-45346 (2004)). These genes are summarized in Table 14 below.

TABLE 14

| | | |
|---|---|---|
| cat1 | P38946.1 | Clostridium kluyveri |
| TVAG_395550 | XP_001330176 | Trichomonas vaginalis G3 |
| Tb11.02.0290 | XP_828352 | Trypanosoma brucei |

The product of the LSC1 and LSC2 genes of S. cerevisiae and the sucC and sucD genes of E. coli naturally form a succinyl-CoA synthetase complex that catalyzes the formation of succinyl-CoA from succinate with the concomitant consumption of one ATP, a reaction which is reversible in vivo (Gruys et al., U.S. Pat. No. 5,958,745, filed Sep. 28, 1999.) Utilization of a succinyl-CoA synthetase instead of a transferase to convert succinate to succinyl-CoA reduces the maximum ATP yield of the MEK synthesis pathway to 1 mol/mol glucose, but does not affect the maximum achievable MEK yield. These genes are summarized below in Table 15.

TABLE 15

| | | |
|---|---|---|
| LSC1 | NP_014785 | Saccharomyces cerevisiae |
| LSC2 | NP_011760 | Saccharomyces cerevisiae |
| sucC | NP_415256.1 | Escherichia coli |
| sucD | AAC73823.1 | Escherichia coli |

Succinyl-CoA can be converted into (R)-methylmalonyl-CoA by methylmalonyl-CoA mutase (MCM). In E. coli, the reversible adenosylcobalamin-dependant mutase participates in a three-step pathway leading to the conversion of succinate to propionate (Dangel et al., Arch. Microbiol. 152:271-279 (1989)). MCM is encoded by genes scpA in Escherichia coli (Bobik and Rasche, Anal. Bioanal. Chem. 375:344-349 (2003); Haller et al., Biochemistry 39:4622-4629 (2000)) and mutA in Homo sapiens (Padovani and Banerjee, Biochemistry 45:9300-9306 (2006)). In several other organisms MCM contains alpha and beta subunits and is encoded by two genes. Exemplary gene candidates encoding the two-subunit protein are Propionibacterium fredenreichii sp. shermani mutA and mutB (Korotkova and Lidstrom, J Biol Chem. 279:13652-13658 (2004)) and Methylobacterium extorquens mcmA and mcmB (Korotkova and Lidstrom, J Biol Chem. 279:13652-13658 (2004)). A summary of the genes involved in the production of (R)-methylmalonyl-CoA is shown below in Table 16.

TABLE 16

| | | |
|---|---|---|
| scpA | NP_417392.1 | Escherichia coli K12 |
| mutA | P22033.3 | Homo sapiens |
| mutA | P11652.3 | Propionibacterium fredenreichii sp. shermanii |
| mutB | P11653.3 | Propionibacterium fredenreichii sp. shermanii |
| mcmA | Q84FZ1 | Methylobacterium extorquens |
| mcmB | Q6TMA2 | Methylobacterium extorquens |

Additional enzymes identified based on high homology to the E. coli spcA gene product that are useful in the practice of the present invention include those listed in Table 17 below.

TABLE 17

| | | |
|---|---|---|
| sbm | NP_838397.1 | Shigella flexneri |
| SARI_04585 | ABX24358.1 | Salmonella enterica |
| YfreA_01000861 | ZP_00830776.1 | Yersinia frederiksenii |

There further exists evidence that genes adjacent to the methylmalonyl-CoA mutase catalytic genes are also required for maximum activity. For example, it has been demonstrated that the meaB gene from M. extorquens forms a complex with methylmalonyl-CoA mutase, stimulates in vitro mutase activity, and possibly protects it from irreversible inactivation (Korotkova and Lidstrom, J Biol Chem. 279:13652-13658 (2004)). The M. extorquens meaB gene product is highly similar to the product of the E. coli argK gene (BLASTp: 45% identity, e-value: 4e-67) which is adjacent to scpA on the chromosome. No sequence for a meaB homolog in P. freudenreichii is catalogued in GenBank. However, the Propionibacterium acnes KPA171202 gene product, YP_{—}055310.1, is 51% identical to the M. extorquens meaB protein and its gene is also adjacent to the methylmalonyl-CoA mutase gene on the chromosome. The relevant genes are shown in Table 18 below.

TABLE 18

| | | |
|---|---|---|
| argK | AAC75955.1 | Escherichia coli K12 |
| | YP_055310.1 | Propionibacterium acnes KPA171202 |
| meaB | 2QM8_B | Methylobacterium extorquens |

Methylmalonyl-CoA epimerase (MMCE) is the enzyme that interconverts (R)-methylmalonyl-CoA and (S)-methylmalonyl-CoA. MMCE is an essential enzyme in the breakdown of odd-numbered fatty acids and of the amino acids valine, isoleucine, and methionine. Methylmalonyl-CoA epimerase is present in organisms such as Bacillus subtilis (YqjC) (Haller et al., Biochemistry 39:4622-4629 (2000)), Homo sapiens (YqjC) (Fuller and Leadlay, Biochem. J 213:643-650 (1983)), Rattus norvegicus (Mcee) (Bobik and Rasche, J Biol Chem. 276:37194-37198 (2001)), Propionibacterium shermanii (AF454511) (Fuller and Leadlay, Biochem. J 213:643-650 (1983); Haller et al., Biochemistry 39:4622-4629 (2000); McCarthy et al., Structure 9:637-646 (2001)) and Caenorhabditis elegans (mmce) (Kuhnl et al., FEBS J 272:1465-1477 (2005)). The additional gene candidates, AE016877 in Bacillus cereus, has high sequence homology to the other characterized enzymes. MMCE activity is required if the employed methylmalonyl-CoA decarboxylase or methylmalonyl-CoA carboxytransferase requires the (S) stereoisomer of methylmalonyl-CoA. The various MMCE genes are summarized below in Table 19.

TABLE 19

| | | |
|---|---|---|
| YqjC | NP_390273 | Bacillus subtilis |
| MCEE | Q96PE7.1 | Homo sapiens |
| Mcee_predicted | NP_001099811.1 | Rattus norvegicus |
| AF454511 | AAL57846.1 | Propionibacterium fredenreichii |
| | | sp. shermanii |
| mmce | AAT92095.1 | Caenorhabditis elegans |
| AE016877 | AAP08811.1 | Bacillus cereus ATCC 14579 |

Methylmalonyl-CoA decarboxylase, is a biotin-independent enzyme that catalyzes the conversion of methylmalonyl-CoA to propionyl-CoA in E. coli (Benning et al., Biochemistry 39:4630-4639 (2000); Haller et al., Biochemistry 39:4622-4629 (2000)). The stereospecificity of the E. coli enzyme was not reported, but Aldor et al. (Aldor et al., Appl Environ.Microbiol 68:3848-3854 (2002)) describe a method of synthesizing propionyl-CoA from succinyl-CoA in Salmonella enterica serovar typhimurium that required only the addition of methylmalonyl-CoA mutase and methylmalonyl-CoA decarboxylase from E. coli. This suggests that the E. coli methylmalonyl-CoA decarboxylase is operative on the (R)-stereoisomer as both organisms, E. coli and S. enterica, are not believed to possess methylmalonyl-CoA epimerase activity. On the other hand, methylmalonyl-CoA decarboxylase from Propionigenium modestum (Bott et al., Eur. J. Biochem. 250:590-599 (1997)) and Veillonella parvula (Huder and Dimroth, J. Biol. Chem. 268:24564-24571 (1993)) catalyze the decarboxylation of the (S)-stereoisomer of methylmalonyl-CoA (Hoffmann and Dimroth, FEBS. Lett. 220:121-125 (1987)). The enzymes from P. modestum and V. parvula are assembled from multiple subunits that not only decarboxylate (S)-methylmalonyl-CoA, but also create a pump that transports sodium ions across the cell membrane as a means to generate energy. The genes for the decarboxylases are summarized below in Table 20.

TABLE 20

| | | |
|---|---|---|
| YgfG | NP_417394 | Escherichia coli |
| mmdA | CAA05137 | Propionigenium modestum |
| mmdD | CAA05138 | Propionigenium modestum |
| mmdC | CAA05139 | Propionigenium modestum |
| mmdB | CAA05140 | Propionigenium modestum |
| mmdA | CAA80872 | Veillonella parvula |
| mmdC | CAA80873 | Veillonella parvula |
| mmdE | CAA80874 | Veillonella parvula |
| mmdD | CAA80875 | Veillonella parvula |
| mmdB | CAA80876 | Veillonella parvula |

Methylmalonyl-CoA carboxytransferase not only catalyzes the conversion of pyruvate to oxaloacetate, but also simultaneously catalyzes the conversion of (S)-methyl-malonyl-CoA to propionyl-CoA (see FIGS. 1 and 2). An exemplary methylmalonyl-CoA carboxytransferase which is comprised of 1.3S, 5S, and 12S subunits can be found in Propionibacterium freudenreichii (Maeda et al. Appl Microbiol Biotechnol 77:879-890 (2007)). The gene information for these subunits is shown below in Table 21.

TABLE 21

| | | |
|---|---|---|
| 1.3S subunit | P02904 | Propionibacterium freudenreichii |
| 5S subunit | Q70AC7 | Propionibacterium freudenreichii |
| 12S subunit | Q8GBW6 | Propionibacterium freudenreichii |

The non-naturally occurring microbial organism of the present invention also has an acetyl-CoA pathway that includes at least one exogenous nucleic acid encoding an acetyl-CoA pathway enzyme expressed in a sufficient amount to produce acetyl-CoA. Such acetyl-CoA pathway enzymes include, for example, a pyruvate kinase, a pyruvate formate lyase, and a formate hydrogen lyase.

Pyruvate formate lyase is an enzyme that catalyzes the conversion of pyruvate and CoA into acetyl-CoA and formate. The reaction can be utilized in the production of MEK from carbohydrates because it allows the biosynthetic pathway to achieve redox balance in the absence of an external electron acceptor. Specifically, the two reducing equivalents generated from forming PEP and pyruvate via glycolysis are consumed by malate dehydrogenase and fumarate reductase coupled to the electron transport chain. Pyruvate formate lyase ensures that an additional reducing equivalent is not formed by the conversion of pyruvate to acetyl-CoA as would be the case if a pyruvate dehydrogenase or pyruvate ferredoxin oxidoreductase enzyme were employed for this transformation. Pyruvate formate lyase is a common enzyme in prokaryotic organisms that is used to help modulate anaerobic redox balance. Exemplary enzymes can be found in Escherichia coli encoded by pflB (Knappe and Sawers, FEMS. Microbiol Rev. 6:383-398 (1990)), Lactococcus lactis (Melchiorsen et al., Appl Microbiol Biotechnol 58:338-344 (2002)), and Streptococcus mutans (Takahashi-Abbe et al., Oral. Microbiol Immunol. 18:293-297 (2003)). E. coli possesses an additional pyruvate formate lyase, encoded by tdcE, that catalyzes the conversion of pyruvate or 2-oxobutanoate to acetyl-CoA or propionyl-CoA, respectively (Hesslinger et al., Mol. Microbiol 27:477-492 (1998)). Both pflB and tdcE from E. coli require the presence of pyruvate formate lyase activating enzyme, encoded by pflA. Further, a short protein encoded by yfiD in E. coli can associate with and restore activity to oxygen-cleaved pyruvate formate lyase (Vey et al., Proc. Natl. Acad. Sci. U.S.A. 105:16137-16141 (2008). Note that pflA and pflB from E. coli were expressed in S. cerevisiae as a means to increase cytosolic acetyl-CoA for butanol production as described in WO/2008/080124]. Additional pyruvate formate lyase and activating enzyme candidates, encoded by pfl and act, respectively, are found in Clostridium pasteurianum (Weidner et al., J Bacteriol. 178:2440-2444 (1996)). A mitochondrial pyruvate formate lyase has also been identified in the eukaryote, Chlamydomonas reinhardtii (Atteia et al., 2006 J. Biol. Chem. 281:9909-9918 (2006); Hemschemeier et al., Eukaryot. Cell 7:518-526 (2008)). Homologous proteins to the E. coli pflA, such as pflA from S. mutans, L. lactis, C. reinhardtii, can be found in many pyruvate formate lyase-containing organisms. A summary of the genes encoding these enzymes is shown below in Table 22.

TABLE 22

| | | |
|---|---|---|
| pflB | NP_415423 | Escherichia coli |
| tdcE | YP_026205 | Escherichia coli |
| pflA | NP_415422 | Escherichia coli |
| yfiD | NP_417074 | Escherichia coli |
| pfl | CAA03993 | Lactococcus lactis |
| pflA | NP_267970 | Lactococcus lactis |
| pfl | NP_720850 | Streptococcus mutans |
| pflA | NP_722023 | Streptococcus mutans |
| PFL1 | EDP09457 | Chlamydomonas reinhardtii |
| pflA | AAW32935 | Chlamydomonas reinhardtii |
| pfl | Q46266.1 | Clostridium pasteurianum |
| act | CAA63749.1 | Clostridium pasteurianum |

A formate hydrogen lyase enzyme can be employed to convert formate to carbon dioxide and hydrogen. An exemplary formate hydrogen lyase enzyme can be found in Escherichia coli. The E. coli formate hydrogen lyase consists of hydrogenase 3 and formate dehydrogenase-H (Maeda et al., Appl Microbiol Biotechnol 77:879-890 (2007)). It is activated by the gene product of fhlA. (Maeda et al., Appl Microbiol Biotechnol 77:879-890 (2007)). The addition of the trace elements, selenium, nickel and molybdenum, to a fermentation broth has been shown to enhance formate hydrogen lyase activity (Soini et al., Microb. Cell Fact. 7:26 (2008)). Various hydrogenase 3, formate dehydrogenase and transcriptional activator genes are shown below in Tables 23 and 24, respectively.

TABLE 23

| | | |
|---|---|---|
| hycD | NP_417202 | Escherichia coli |
| hycC | NP_417203 | Escherichia coli |
| hycF | NP_417200 | Escherichia coli |
| hycG | NP_417199 | Escherichia coli |
| hycB | NP_417204 | Escherichia coli |
| hycE | NP_417201 | Escherichia coli |

TABLE 24

| | | |
|---|---|---|
| fdhF | NP_418503 | Escherichia coli |
| fhlA | NP_417211 | Escherichia coli |

A formate hydrogen lyase enzyme also exists in the hyperthermophilic archaeon, Thermococcus litoralis (Takacs et al., BMC. Microbiol 8:88 (2008)). Exemplary genes from T. litoralis are provided in Table 25 below.

TABLE 25

| | | |
|---|---|---|
| mhyC | ABW05543 | Thermococcus litoralis |
| mhyD | ABW05544 | Thermococcus litoralis |
| mhyE | ABW05545 | Thermococcus litoralis |
| myhF | ABW05546 | Thermococcus litoralis |
| myhG | ABW05547 | Thermococcus litoralis |
| myhH | ABW05548 | Thermococcus litoralis |
| fdhA | AAB94932 | Thermococcus litoralis |
| fdhB | AAB94931 | Thermococcus litoralis |

Additional formate hydrogen lyase systems have been found in Salmonella typhimurium, Klebsiella pneumoniae, Rhodospirillum rubrum, Methanobacterium formicicum (Vardar-Schara et al., Microbial Biotechnology 1:107-125 (2008)).

In some embodiments, the present invention also provides a non-naturally occurring microbial organism that includes a microbial organism having a 2-butanol pathway. This pathway at least one exogenous nucleic acid encoding a 2-butanol pathway enzyme expressed in a sufficient amount to produce 2-butanol. The pathway includes many enzymes found in the MEK pathway such as a β-ketothiolase, a β-ketovalerate decarboxylase, and at least one of a β-ketovaleryl-CoA hydrolase and a β-ketovaleryl -CoA transferase. The final enzyme in the pathway facilitating reduction of MEK is a methyl ethyl ketone reductase.

The non-naturally occurring microbial organisms that produce 2-butanol include most of the enzymes used in the production of MEK from acetyl-CoA and propionyl-CoA with the exception of formate hydrogen lyase (See FIGS. 3 and 4). Instead, formate is converted to carbon dioxide by a formate dehydrogenase that provides the additional reducing equivalent used in 2-butanol synthesis from MEK. Alternatively, this reducing equivalent is obtained by using pyruvate dehydrogenase or pyruvate ferredoxin oxidoreductase as shown further below.

The requisite methyl ethyl ketone reductase, or alternatively, 2-butanol dehydrogenase, catalyzes the reduction of MEK to form 2-butanol. Exemplary enzymes can be found in Rhodococcus ruber (Kosjek et al., Biotechnol Bioeng. 86:55-62 (2004)) and Pyrococcus furiosus (van der et al., Eur. J. Biochem. 268:3062-3068 (2001)). Additional secondary alcohol dehydrogenase enzymes capable of this transformation include adh from C. beijerinckii (Hanai et al., Appl Environ Microbiol 73:7814-7818 (2007); Jojima et al., Appl Microbiol Biotechnol 77:1219-1224 (2008)) and adh from Thermoanaerobacter brockii (Hanai et al., Appl Environ Microbiol 73:7814-7818 (2007); Peretz et al., Anaerobe 3:259-270 (1997)). The summary of these genes is shown in Table 26 below.

TABLE 26

| | | |
|---|---|---|
| sadh | CAD36475 | Rhodococcus rubber |
| adhA | AAC25556 | Pyrococcus furiosus |
| Adh | P14941.1 | Thermoanaerobobacter brockii |
| Adh | AAA23199.2 | Clostridium beijerinckii |

The non-naturally occurring microbial organisms of the present invention that produce 2-butanol also have a propionyl-CoA pathway that includes at least one exogenous nucleic acid encoding a propionyl-CoA pathway enzyme expressed in a sufficient amount to produce propionyl-CoA. The pathway enzymes include, for example, those of the propionyl-CoA pathway used in MEK biosynthesis such as any combination of a PEP carboxylase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

Likewise, the non-naturally occurring microbial organism of the present invention that produce 2-butanol also have an acetyl-CoA pathway that includes at least one exogenous nucleic acid encoding an acetyl-CoA pathway enzyme expressed in a sufficient amount to produce acetyl-CoA. The acetyl-CoA pathway enzymes include, for example, any combination of a pyruvate kinase and either a pyruvate formate lyase and a formate dehydrogenase or an enzyme selected from the group consisting of a pyruvate dehydrogenase and a pyruvate ferredoxin oxidoreductase.

Saccharomyces cerevisiae contains two formate dehydrogenases, FDH1 and FDH2, that catalyze the oxidation of formate to CO₂ (Overkamp et al., Yeast 19:509-520 (2002)). In Moorella thermoacetica, the loci, Moth_{—}2312 and Moth_{—}2313, are actually one gene that is responsible for encoding the alpha subunit of formate dehydrogenase while the beta subunit is encoded by Moth_{—}2314 (Andreesen and Ljungdahl, J. Bacteriol. 116:867-873 (1973); Li et al., J. Bacteriol. 92:405-412 (1966); Pierce et al., Environ. Microbiol (2008); Yamamoto et al., J. Biol. Chem. 258:1826-1832 (1983)). Another set of genes encoding formate dehydrogenase activity is encoded by Sfum_{—}2703 through Sfum_{—}2706 in Syntrophobacter fumaroxidans (de Bok et al., Eur. J. Biochem. 270:2476-2485 (2003); Reda et al., Proc. Natl. Acad. Sci. U S.A. 105:10654-10658 (2008)). Similar to their M. thermoacetica counterparts, Sfum_{—}2705 and Sfum_{—}2706 are actually one gene. A summary of these genes is provided in Table 27 below.

TABLE 27

| | | |
|---|---|---|
| FDH1 | NP_015033 | Saccharomyces cerevisiae |
| FDH2 | Q08987 | Saccharomyces cerevisiae |
| Moth_2312 | YP_431142 | Moorella thermoacetica |
| Moth_2313 | YP_431143 | Moorella thermoacetica |
| Moth_2314 | YP_431144 | Moorella thermoacetica |
| Sfum_2703 | YP_846816.1 | Syntrophobacter fumaroxidans |
| Sfum_2704 | YP_846817.1 | Syntrophobacter fumaroxidans |
| Sfum_2705 | YP_846818.1 | Syntrophobacter fumaroxidans |
| Sfum_2706 | YP_846819.1 | Syntrophobacter fumaroxidans |

The pyruvate dehydrogenase complex, catalyzing the conversion of pyruvate to acetyl-CoA, has been studied. The S. cerevisiae complex consists of an E2 (LAT1) core that binds E1 (PDA1, PDB1), E3 (LPD1), and Protein X (PDX1) components (Pronk et al., Yeast 12:1607-1633 (1996)). In the E. coli enzyme, specific residues in the E1 component are responsible for substrate specificity (Bisswanger, H., J Biol Chem. 256:815-822 (1981); Bremer, J. Eur. J Biochem. 8:535-540 (1969); Gong et al., J Biol Chem. 275:13645-13653 (2000)). Engineering efforts have improved the E. coli PDH enzyme activity under anaerobic conditions (Kim et al., Appl. Environ. Microbiol. 73:1766-1771 (2007)); Kim et al., J. Bacteriol. 190:3851-3858 (2008); Zhou et al., Biotechnol. Lett. 30:335-342 (2008)). In contrast to the E. coli PDH, the B. subtilis complex is active and required for growth under anaerobic conditions (Nakano et al., J. Bacteriol. 179:6749-6755 (1997)). The Klebsiella pneumoniae PDH, characterized during growth on glycerol, is also active under anaerobic conditions (Menzel et al., J. Biotechnol. 56:135-142 (1997)). Crystal structures of the enzyme complex from bovine kidney (Zhou et al., Proc. Natl. Acad. Sci. U.S.A 98:14802-14807 (2000)) and the E2 catalytic domain from Azotobacter vinelandii are available (Mattevi et al., Science. 255:1544-1550 (1992)). Some mammalian PDH enzymes complexes can react on alternate substrates such as 2-oxobutanoate (Paxton et al., Biochem. J. 234:295-303 (1986)). A summary of these genes is provided below in Table 28.

TABLE 28

| | | |
|---|---|---|
| LAT1 | NP_014328 | Saccharomyces cerevisiae |
| PDA1 | NP_011105 | Saccharomyces cerevisiae |
| PDB1 | NP_009780 | Saccharomyces cerevisiae |
| LPD1 | NP_116635 | Saccharomyces cerevisiae |
| PDX1 | NP_011709 | Saccharomyces cerevisiae |
| aceE | NP_414656.1 | Escherichia coli str. K12 substr. MG1655 |
| aceF | NP_414657.1 | Escherichia coli str. K12 substr. MG1655 |
| lpd | NP_414658.1 | Escherichia coli str. K12 substr. MG1655 |
| pdhA | P21881.1 | Bacillus subtilis |
| pdhB | P21882.1 | Bacillus subtilis |
| pdhC | P21883.2 | Bacillus subtilis |
| pdhD | P21880.1 | Bacillus subtilis |
| aceE | YP_001333808.1 | Klebsiella pneumonia MGH78578 |
| aceF | YP_001333809.1 | Klebsiella pneumonia MGH78578 |
| lpdA | YP_001333810.1 | Klebsiella pneumonia MGH78578 |
| Pdha1 | NP_001004072.2 | Rattus norvegicus |
| Pdha2 | NP_446446.1 | Rattus norvegicus |
| Dlat | NP_112287.1 | Rattus norvegicus |
| Dld | NP_955417.1 | Rattus norvegicus |

Pyruvate ferredoxin oxidoreductase (PFOR) catalyzes the oxidation of pyruvate to form acetyl-CoA. The PFOR from Desulfovibrio africanus has been cloned and expressed in E. coli resulting in an active recombinant enzyme that was stable for several days in the presence of oxygen (Pieulle et al., J Bacteriol. 179:5684-5692 (1997)). Oxygen stability is relatively uncommon in PFORs and is reported to be conferred by a 60 residue extension in the polypeptide chain of the D. africanus enzyme. The M. thermoacetica PFOR is also well characterized (Menon and Ragsdale, Biochemistry 36:8484-8494 (1997)) and was even shown to have high activity in the direction of pyruvate synthesis during autotrophic growth (Furdui and Ragsdale, J Biol Chem. 275:28494-28499 (2000)). Further, E. coli possesses an uncharacterized open reading frame, ydbK, that encodes a protein that is 51% identical to the M. thermoacetica PFOR. Evidence for pyruvate oxidoreductase activity in E. coli has been described (Blaschkowski et al., Eur. J Biochem. 123:563-569 (1982)). Several additional PFOR enzymes are described in the following review (Ragsdale, S. W., Chem. Rev. 103:2333-2346 (2003)). Finally, flavodoxin reductases (e.g., fqrB from Helicobacter pylori or Campylobacter jejuni (St. Maurice et al., J. Bacteriol. 189:4764-4773) (2007)) or Rnf-type proteins (Herrmann et al., J. Bacteriol. 190:784-791 (2008); Seedorf et al., Proc. Natl. Acad. Sci. U S.A. 105:2128-2133 (2008)) provide a means to generate NADH or NADPH from the reduced ferredoxin generated by PFOR. A summary of these genes is provided in Table 29 below.

TABLE 29

| | | |
|---|---|---|
| por | CAA70873.1 | Desulfovibrio africanus |
| por | YP_428946.1 | Moorella thermoacetica |
| ydbK | NP_415896.1 | Escherichia coli |
| fqrB | NP_207955.1 | Helicobacter pylori |
| fqrB | YP_001482096.1 | Campylobacter jejuni |
| RnfC | EDK33306.1 | Clostridium kluyveri |
| RnfD | EDK33307.1 | Clostridium kluyveri |
| RnfG | EDK33308.1 | Clostridium kluyveri |
| RnfE | EDK33309.1 | Clostridium kluyveri |
| RnfA | EDK33310.1 | Clostridium kluyveri |
| RnfE | EDK33311.1 | Clostridium kluyveri |

In some embodiments, the present invention provides a non-naturally occurring microbial organism that includes a microbial organism having an alternative methyl ethyl ketone pathway comprising at least one exogenous nucleic acid encoding a methyl ethyl ketone pathway enzyme expressed in a sufficient amount to produce methyl ethyl ketone. The alternative methyl ethyl ketone pathway includes a 2-methylacetoacetyl-CoA thiolase, a 2-methylacetoacetate decarboxylase and at least one of a 2-methylacetoacetyl-CoA hydrolase and a 2-methylacetoacetyl-CoA transferase.

The first step of in this alternate pathway entails the conversion of propionyl-CoA and acetyl-CoA to 2-methylacetoacetyl-CoA. The subsequent conversion of 2-methylacetoacetyl-CoA to MEK is catalyzed by enzymes exhibiting similar chemistries as described herein above for converting β-ketovaleryl-CoA to MEK. The energetic yields and redox balances of the two pathways are similar.

Human mitochondrial 2-methylacetoacetyl-CoA thiolase deficiency has been reportedly linked to urinary excretion of 2-methyl-3-hydroxybutyric acid, tiglylglycine, and in some instances also 2-methyl-acetoacetic acid (Sovik, O., J. Inherit. Metab. Dis. 16:46-54 (1993)). The 2-methylacetoacetyl-CoA thiolase gene has been cloned and sequenced (Sovik, O. supra). Pseudomonas putida also oxidizes isoleucine to acetyl-CoA and propionyl-CoA by a pathway that passes through 2-methylacetoacetyl-CoA (Conrad et al., J. Bacteriol. 118:103-111 (1974). Given its proximity on the P. putida chromosome to fadBx, a gene likely to encode 3-hydroxy-2-methylbutyryl-CoA dehydrogenase based on its high sequence homology to the known human gene HADH2 (Ofman et al., Am. J. Hum. Genet. 72:1300-1307 (2003)), the gene fadAx likely encodes 2-methylacetoacetyl-CoA thiolase.

Ascaris lumbricoides has been shown to produce alpha-methylbutyric acid (Bueding and Yale, J. Biol.Chem. 193:411-423 (1951)) directly from the precursors acetate and propionate (Saz and Weil, J. Biol. Chem. 235:914-918 (1960)) indicating that a thiolase forms 2-methylacetoacetyl-CoA from acetyl-CoA and propionyl-CoA. The sequence of the gene encoding 2-methylacetoacetyl-CoA thiolase has not been reported although the kinetics of the enzyme in Ascaris suum have been studied (Suarez et al. 1991 Arch. Biochem. Biophys. 285:166-171 (1991); Suarez et al., Arch. Biochem. Biophys. 285:158-165 (1991)). An EST database for Ascaris suum is available on the world wide web at Nematode.net (Martin et al., 2008 Nucleic. Acids Res. (2008); Wylie et al., Nucleic. Acids Res. 32:D423-D426 (2004). The DNA sequence encoding the enzyme responsible for the thiolase activity can be isolated from an A. suum cDNA library using probes. Such a cDNA library can be constructed from A. suum mRNA according to general molecular biology practice. The probes can be designed with whole or partial DNA sequences from the following EST sequences from the publically available Nematode.net database which were obtained based on sequence homology to the human thiolase: AS02764, AS02560, AS 13583, AS00875, AS10248. The A. suum cDNA library can be screened with the probes derived from these EST sequences, and the resulting cDNA clones can be sequenced. The DNA sequences generated from this process can then be used for transformation into S. cerevisiae or any other organism. An additional candidate thiolase from Caenorhabditis elegans can be identified based on homology to AS02764, the most similar A. suum EST to the human gene, ACAT1. A summary of these genes are provided below in Table 30.

TABLE 30

| | | |
|---|---|---|
| ACAT1 | NP_000010 | Homo sapiens |
| fadAx | AAK18171 | Pseudomonas putida |
| kat-1 | NP_495455 | Caenorhabditis elegans |

The conversion of 2-methylacetoacetyl-CoA to 2-methylacetoacetate can be carried out by 2-methylacetoacetyl-CoA transferase which conserves the energy stored in the CoA-ester bond. One enzyme for this reaction step is succinyl-CoA:3-ketoacid-CoA transferase. This enzyme converts succinate to succinyl-CoA while converting a 3-ketoacyl-CoA to a 3-ketoacid. This enzyme is useful not only for converting 2-methylacetoacetyl-CoA to 2-methylacetoacetate, but also for catalyzing the conversion of succinate to succinyl-CoA (see FIG. 2). Exemplary succinyl-CoA:3:ketoacid-CoA transferases are present in Helicobacter pylori (Corthesy-Theulaz et al., J. Biol. Chem. 272:25659-25667 (1997)), Bacillus subtilis (Stols et al., Protein. Expr. Purif. 53:396-403 (2007)), and Homo sapiens (Fukao et al., Genomics 68:144-151 (2000); Takahashi-Abbe et al., Oral. Microbiol Immunol. 18:293-297 (2003)). A summary of these genes are provided in Table 31 below.

TABLE 31

| | | |
|---|---|---|
| HPAG1_0676 | YP_627417 | Helicobacter pylori |
| HPAG1_0677 | YP_627418 | Helicobacter pylori |
| ScoA | NP_391778 | Bacillus subtilis |
| ScoB | NP_391777 | Bacillus subtilis |
| OXCT1 | NP_000427 | Homo sapiens |
| OXCT2 | NP_071403 | Homo sapiens |

2-methylacetoacetyl-CoA can be hydrolyzed to 2-methylacetoacetate by 2-methylacetoacetyl-CoA hydrolase Using such an enzyme reduces the maximum ATP yield of the overall MEK pathway to 1 mol ATP /mol glucose, but does not reduce the maximum theoretical yield of MEK. Several eukaryotic acetyl-CoA hydrolases (EC 3.1.2.1) have broad substrate specificity. The enzyme from Rattus norvegicus brain (131) can react with butyryl-CoA, hexanoyl-CoA and malonyl-CoA. Though its sequence has not been reported, the enzyme from the mitochondrion of the pea leaf showed activity on acetyl-CoA, propionyl-CoA, butyryl-CoA, palmitoyl-CoA, oleoyl-CoA, succinyl-CoA, and crotonyl-CoA (Zeiher and Randall, Plant. Physiol. 94:20-27 (1990)). Additionally, a glutaconate CoA-transferase from Acidaminococcus fermentans was transformed by site-directed mutagenesis into an acyl-CoA hydrolase with activity on glutaryl-CoA, acetyl-CoA and 3-butenoyl-CoA (Mack and Buckel, FEBS. Lett. 405:209-212 (1997)). This indicates that the enzymes encoding succinyl-CoA:3-ketoacid-CoA transferases and acetoacetyl-CoA:acetyl-CoA transferases can also serve for this reaction step with certain mutations to change their function. The acetyl-CoA hydrolase, ACH1, from S. cerevisiae represents another candidate hydrolase (Buu et al., J. Biol. Chem. 278:17203-17209 (2003)). A summary of these genes is provided in Table 32 below.

TABLE 32

| | | |
|---|---|---|
| acot12 | NP_570103.1 | Rattus norvegicus |
| gctA | CAA57199 | Acidaminococcus fermentans |
| gctB | CAA57200 | Acidaminococcus fermentans |
| ACH1 | NP_009538 | Saccharomyces cerevisiae |

The acetoacetate decarboxylase enzymes described herein above can exhibit activity on 2-methylacetoacetate as well. In alternative embodiments an enzyme having this decarboxylase activity is α-acetolactate decarboxylase that converts α-acetolactate to acetoin. The difference between α-acetolactate and 2-methylacetoacetate from a structural standpoint is the presence of a hydroxy group on the 2-carbon of α-acetolactate. Exemplary α-acetolactate decarboxylase enzymes have been identified in Acetobacter aceti (Yamano et al., J. Biotechnol 32:173-178 (1994)), Enterobacter aerogenes (Sone et al., Appl Environ. Microbiol 54:38-42 (1988)), Raoultella terrigena (Blomqvist et al., J. Bacteriol. 175:1392-1404, (1993)) among many other organisms. The relevant genes for this transformation are shown below in Table 33.

TABLE 33

| | | |
|---|---|---|
| ALDC | AAC60472 | Acetobacter aceti |
| aldC | P05361 | Enterobacter aerogenes |
| budA | Q04518 | Raoultella terrigena |

The non-naturally occurring microbial organism of the present invention having the alternate pathway through 2-methylacetoacetate also has a propionyl-CoA pathway that includes at least one exogenous nucleic acid encoding a propionyl-CoA pathway enzyme expressed in a sufficient amount to produce propionyl-CoA, as described herein above, including any combination of a PEP carboxylase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

Likewise, the non-naturally occurring microbial organism having the MEK pathway through 2-methylacetoacetate also includes an acetyl-CoA pathway that has at least one exogenous nucleic acid encoding an acetyl-CoA pathway enzyme expressed in a sufficient amount to produce acetyl-CoA, as described above, including a pyruvate kinase, a pyruvate formate lyase, and a formate hydrogen lyase.

In some embodiments, a non-naturally occurring microbial organism that has an MEK pathway via 2-methylacetoacetate can also be further engineered to produce 2-butanol. Such a microbial organism has a 2-butanol pathway including at least one exogenous nucleic acid encoding a 2-butanol pathway enzyme expressed in a sufficient amount to produce 2-butanol. As before, the 2-butanol pathway includes a 2-methylacetoacetyl-CoA thiolase, a 2-methylacetoacetate decarboxylase, a methyl ethyl ketone reductase and at least one of a 2-methylacetoacetyl-CoA hydrolase, and a 2-methylacetoacetyl-CoA transferase.

The non-naturally occurring microbial organism of the present invention that produce 2-butanol through the 2-methylacetoacetate pathway also possess a propionyl-CoA pathway that includes at least one exogenous nucleic acid encoding a propionyl-CoA pathway enzyme expressed in a sufficient amount to produce propionyl-CoA, as previously described, as well as an acetyl-CoA pathway that includes at least one exogenous nucleic acid encoding an acetyl-CoA pathway enzyme expressed in a sufficient amount to produce acetyl-CoA. Again the acetyl-CoA pathway enzyme includes any combination of a pyruvate kinase and either a pyruvate formate lyase and a formate dehydrogenase, or an enzyme selected from a pyruvate dehydrogenase and a pyruvate ferredoxin oxidoreductase.

In some embodiments, the present invention provides a non-naturally occurring microbial organism that includes a microbial organism having a propionyl-CoA pathway with at least one exogenous nucleic acid encoding a propionyl-CoA pathway enzyme expressed in a sufficient amount to produce propionyl-CoA. The propionyl-CoA pathway includes a methylmalonyl-CoA mutase, a methylmalonyl-CoA epimerase, and at least one of a methylmalonyl-CoA decarboxylase and a methylmalonyl-CoA carboxytransferase. Such an organism also includes at least one propionyl-CoA pathway enzyme selected from a PEP carboxylase, a pyruvate carboxylase, a methylmalonyl-CoA carboxytransferase, a malate dehydrogenase, a fumarase, a fumarate reductase, a succinyl-CoA transferase and a succinyl-CoA synthetase.

As mentioned herein above, propionyl-CoA can also be produced by way of threonine. Thus, in some embodiments the invention provides a non-naturally occurring microbial organism that includes a microbial organism having a methyl ethyl ketone pathway. The pathway includes at least one exogenous nucleic acid encoding a methyl ethyl ketone pathway enzyme expressed in a sufficient amount to produce methyl ethyl ketone. The methyl ethyl ketone pathway includes a β-ketothiolase, a β-ketovalerate decarboxylase and an enzyme selected from a β-ketovaleryl-CoA hydrolase, and a β-ketovaleryl-CoA transferase. The methyl ethyl ketone pathway further includes a propionyl-CoA pathway having a threonine deaminase.

In other embodiments, the invention provides a non-naturally occurring microbial organism that includes a microbial organism having a 2-butanol pathway. The pathway includes at least one exogenous nucleic acid encoding a 2-butanol pathway enzyme expressed in a sufficient amount to produce 2-butanol. The 2-butanol pathway includes a β-ketothiolase, a β-ketovalerate decarboxylase, a methyl ethyl ketone reductase and an enzyme selected from a β-ketovaleryl-CoA hydrolase and a β-ketovaleryl -CoA transferase. The 2-butanol pathway further includes a propionyl-CoA pathway having a threonine deaminase.

In yet further embodiments, the invention provides a non-naturally occurring microbial organism that includes a microbial organism having a methyl ethyl ketone pathway. The pathway includes at least one exogenous nucleic acid encoding a methyl ethyl ketone pathway enzyme expressed in a sufficient amount to produce methyl ethyl ketone. The methyl ethyl ketone pathway includes a 2-methylacetoacetyl-CoA thiolase, a 2-methylacetoacetate decarboxylase and an enzyme selected from a 2-methylacetoacetyl-CoA hydrolase and a 2-methylacetoacetyl-CoA transferase. The methyl ethyl ketone pathway further includes a propionyl-CoA pathway having a threonine deaminase.

In still further embodiments, the invention provides a non-naturally occurring microbial organism that includes a microbial organism having a 2-butanol pathway. The pathway includes at least one exogenous nucleic acid encoding a 2-butanol pathway enzyme expressed in a sufficient amount to produce 2-butanol. The 2-butanol pathway includes a 2-methylacetoacetyl-CoA thiolase, a 2-methylacetoacetate decarboxylase, a methyl ethyl ketone reductase and an enzyme selected from a 2-methylacetoacetyl-CoA hydrolase and a 2-methylacetoacetyl-CoA transferase. The 2-butanol pathway further includes a propionyl-CoA pathway having a threonine deaminase.

In accordance with embodiments in which propionyl-CoA is generated from threonine, as exemplified in FIG. 5, the first step en route to propionyl-CoA is the conversion of threonine to 2-ketobutyrate by action of a threonine deaminase. In some embodiments, the threonine deaminase is encoded by one or more genes selected from ilvA (Calhoun et al. J. Biol. Chem. 248(10):3511-6, (1973)) and tdcB (Umbarger et al. J. Bacteriol. 73(1):105-12, (1957); Datta et al. Proc. Natl. Acad. Sci. U S A 84(2): 393-7(1987)). Rhodospirillum rubrum represents an additional exemplary organism containing threonine deaminase (Feldberg et al. Eur. J. Biochem. 21(3): 438-46 (1971); U.S. Pat. No. 5,958,745). Details for exemplary enzymes for carrying out this transformation are shown below in Table 34.

TABLE 34

| | | |
|---|---|---|
| ilvA | AAC77492 | Escherichia coli |
| tdcB | AAC76152 | Escherichia coli |
| Rru_A2877 | YP_427961.1 | Rhodospirillum rubrum |
| Rru_A0647 | YP_425738.1 | Rhodospirillum rubrum |

2-ketobutyrate is then converted to propionyl-CoA via a pyruvate formate lyase and a pyruvate formate lyase activating enzyme. The pyruvate formate lyase is encoded by gene selected from pflB and tdcE, while the pyruvate formate lyase activating enzyme is encoded by a pflA gene. Details for these exemplary genes for carrying out this transformation are shown above in Table 22.

Alternatively, 2-ketobutyrate can be converted to propionyl-CoA by means of pyruvate dehydrogenase, pyruvate ferredoxin oxidoreductase (PFOR), or any other enzyme with 2-ketoacid dehydrogenase functionality. Such enzymes are also capable of converting pyruvate to acetyl-CoA. Exemplary pyruvate dehydrogenase enzymes are present in E. coli (Bisswanger, H., J. Biol. Chem. 256:815-822 (1981); Bremer, J. Eur. J. Biochem. 8:535-540 (1969); Gong et al., J. Biol. Chem. 275:13645-13653 (2000)), B. subtilis (Nakano et al., J. Bacteriol. 179:6749-6755 (1997)), K. pneumonia (Menzel et al., J. Biotechnol. 56:135-142 (1997)), R. norvegicus (Paxton et al., Biochem. J. 234:295-303 (1986)), for example. Exemplary gene information is provided in Table 35 below.

TABLE 35

| | | |
|---|---|---|
| aceE | NP_414656.1 | Escherichia coli str. K12 substr. MG1655 |
| aceF | NP_414657.1 | Escherichia coli str. K12 substr. MG1655 |
| lpd | NP_414658.1 | Escherichia coli str. K12 substr. MG1655 |
| pdhA | P21881.1 | Bacillus subtilis |
| pdhB | P21882.1 | Bacillus subtilis |
| pdhC | P21883.2 | Bacillus subtilis |
| pdhD | P21880.1 | Bacillus subtilis |
| aceE | YP_001333808.1 | Klebsiella pneumonia MGH78578 |
| aceF | YP_001333809.1 | Klebsiella pneumonia MGH78578 |
| lpdA | YP_001333810.1 | Klebsiella pneumonia MGH78578 |
| Pdha1 | NP_001004072.2 | Rattus norvegicus |
| Pdha2 | NP_446446.1 | Rattus norvegicus |
| Dlat | NP_112287.1 | Rattus norvegicus |
| Dld | NP_955417.1 | Rattus norvegicus |

Exemplary PFOR enzymes include, for example, the enzyme from Desulfovibrio africanus which has been cloned and expressed in E. coli, resulting in an active recombinant enzyme that was stable for several days in the presence of oxygen (Pieulle et al., J. Bacteriol. 179:5684-5692 (1997)). Oxygen stability is relatively uncommon in PFORs and is reported to be conferred by a 60 residue extension in the polypeptide chain of the D. africanus enzyme. The M. thermoacetica PFOR is also well characterized (Menon et al. Biochemistry 36:8484-8494 (1997)) and was shown to have high activity in the direction of pyruvate synthesis during autotrophic growth (Furdui et al. J. Biol. Chem. 275:28494-28499 (2000)). Further, E. coli possesses an uncharacterized open reading frame, ydbK, that encodes a protein that is 51% identical to the M. thermoacetica PFOR. Evidence for pyruvate oxidoreductase activity in E. coli has been described (Blaschkowski et al., Eur. J. Biochem. 123:563-569 (1982)). The protein sequences of these exemplary PFOR enzymes can be identified by the following GenBank accession numbers as shown in Table 36 below. Several additional PFOR enzymes have been described (Ragsdale, Chem. Rev. 103:2333-2346 (2003)).

TABLE 36

| | | |
|---|---|---|
| Por | CAA70873.1 | Desulfovibrio africanus |
| Por | YP_428946.1 | Moorella thermoacetica |
| YdbK | NP_415896.1 | Escherichia coli |

Additional routes for producing propionyl-CoA are disclosed in US 5958745 which is incorporated by reference herein in its entirety. One such route involves converting 2-ketobutyrate to propionate by pyruvate oxidase, and converting propionate to propionyl-CoA via an acyl-CoA synthetase.

In still further embodiments, the present invention provides a non-naturally occurring microbial organism that includes a microbial organism having an acetyl-CoA pathway with at least one exogenous nucleic acid encoding an acetyl-CoA pathway enzyme expressed in a sufficient amount to produce acetyl-CoA. The acetyl-CoA pathway can include a pyruvate kinase, a pyruvate formate lyase and a formate hydrogen lyase.

In further embodiments, the present invention provides a non-naturally occurring microbial organism that includes a microbial organism having an acetyl-CoA pathway with at least one exogenous nucleic acid encoding an acetyl-CoA pathway enzyme expressed in a sufficient amount to produce acetyl-CoA. The acetyl-CoA pathway can include a pyruvate kinase, a pyruvate formate lyase, a formate dehydrogenase and at least one of a pyruvate dehydrogenase and a pyruvate ferredoxin oxidoreductase.

The invention is described herein with general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms of the invention can be produced by introducing expressible nucleic acids encoding one or more of the enzymes or proteins participating in one or more methyl ethyl ketone and/or 2-butanol biosynthetic pathways. Depending on the host microbial organism chosen for biosynthesis, nucleic acids for some or all of a particular methyl ethyl ketone and/or 2-butanol biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve methyl ethyl ketone and/or 2-butanol biosynthesis. Thus, a non-naturally occurring microbial organism of the invention can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as methyl ethyl ketone and/or 2-butanol.

Depending on the methyl ethyl ketone and/or 2-butanol biosynthetic pathway constituents of a selected host microbial organism, the non-naturally occurring microbial organisms of the invention will include at least one exogenously expressed methyl ethyl ketone and/or 2-butanol pathway-encoding nucleic acid and up to all encoding nucleic acids for one or more methyl ethyl ketone and/or 2-butanol biosynthetic pathways. For example, methyl ethyl ketone and/or 2-butanol biosynthesis can be established in a host deficient in a pathway enzyme or protein through exogenous expression of the corresponding encoding nucleic acid. In a host deficient in all enzymes or proteins of a methyl ethyl ketone and/or 2-butanol pathway, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of methyl ethyl ketone and/or 2-butanol can be included.

Given the teachings and guidance provided herein, those skilled in the art will understand that the number of encoding nucleic acids to introduce in an expressible form will, at least, parallel the methyl ethyl ketone and/or 2-butanol pathway deficiencies of the selected host microbial organism. Therefore, a non-naturally occurring microbial organism of the invention can have one, two, three, four, five, six, seven, eight, nine, ten, up to all nucleic acids encoding the enzymes or proteins constituting a methyl ethyl ketone and/or 2-butanol biosynthetic pathway disclosed herein. In some embodiments, the non-naturally occurring microbial organisms also can include other genetic modifications that facilitate or optimize methyl ethyl ketone and/or 2-butanol biosynthesis or that confer other useful functions onto the host microbial organism. One such other functionality can include, for example, augmentation of the synthesis of one or more of the methyl ethyl ketone and/or 2-butanol pathway precursors such as beta-ketovalerate or 2-methylacetoacetate for methyl ethyl ketone production or methyl ethyl ketone itself, in the production of 2-butanol.

Generally, a host microbial organism is selected such that it produces the precursor of a methyl ethyl ketone and/or 2-butanol pathway, either as a naturally produced molecule or as an engineered product that either provides de novo production of a desired precursor or increased production of a precursor naturally produced by the host microbial organism. For example, methyl ethyl ketone and/or 2-butanol may be produced naturally in a host organism. A host organism can be engineered to increase production of a precursor, as disclosed herein. In addition, a microbial organism that has been engineered to produce a desired precursor can be used as a host organism and further engineered to express enzymes or proteins of a methyl ethyl ketone and/or 2-butanol pathway.

In some embodiments, a non-naturally occurring microbial organism of the invention is generated from a host that contains the enzymatic capability to synthesize methyl ethyl ketone and/or 2-butanol. In this specific embodiment it can be useful to increase the synthesis or accumulation of a methyl ethyl ketone and/or 2-butanol pathway product to, for example, drive methyl ethyl ketone and/or 2-butanol pathway reactions toward methyl ethyl ketone and/or 2-butanol production. Increased synthesis or accumulation can be accomplished by, for example, overexpression of nucleic acids encoding one or more of the above-described methyl ethyl ketone and/or 2-butanol pathway enzymes or proteins. Overexpression the enzyme or enzymes and/or protein or proteins of the methyl ethyl ketone and/or 2-butanol pathway can occur, for example, through exogenous expression of the endogenous gene or genes, or through exogenous expression of the heterologous gene or genes. Therefore, naturally occurring organisms can be readily generated to be non-naturally occurring microbial organisms of the invention, for example, producing methyl ethyl ketone and/or 2-butanol, through overexpression of one, two, three, four, five, six, seven, eight, nine, 10, that is, up to all nucleic acids encoding methyl ethyl ketone and/or 2-butanol biosynthetic pathway enzymes or proteins. In addition, a non-naturally occurring organism can be generated by mutagenesis of an endogenous gene that results in an increase in activity of an enzyme in the methyl ethyl ketone and/or 2-butanol biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods of the invention, any of the one or more exogenous nucleic acids can be introduced into a microbial organism to produce a non-naturally occurring microbial organism of the invention. The nucleic acids can be introduced so as to confer, for example, a methyl ethyl ketone and/or 2-butanol biosynthetic pathway onto the microbial organism. Alternatively, encoding nucleic acids can be introduced to produce an intermediate microbial organism having the biosynthetic capability to catalyze some of the required reactions to confer methyl ethyl ketone and/or 2-butanol biosynthetic capability. For example, a non-naturally occurring microbial organism having a methyl ethyl ketone and/or 2-butanol biosynthetic pathway can comprise at least two exogenous nucleic acids encoding desired enzymes or proteins, such as the combination of methyl ethyl ketone and/or 2-butanol, and the like. Thus, it is understood that any combination of two or more enzymes or proteins of a biosynthetic pathway can be included in a non-naturally occurring microbial organism of the invention. Similarly, it is understood that any combination of three or more enzymes or proteins of a biosynthetic pathway can be included in a non-naturally occurring microbial organism of the invention, so long as the combination of enzymes and/or proteins of the desired biosynthetic pathway results in production of the corresponding desired product. Similarly, any combination of four, five, six, seven, eight, nine, ten, or more enzymes or proteins of a biosynthetic pathway as disclosed herein can be included in a non-naturally occurring microbial organism of the invention, as desired, so long as the combination of enzymes and/or proteins of the desired biosynthetic pathway results in production of the corresponding desired product.

In addition to the biosynthesis of methyl ethyl ketone and/or 2-butanol as described herein, the non-naturally occurring microbial organisms and methods of the invention also can be utilized in various combinations with each other and with other microbial organisms and methods well known in the art to achieve product biosynthesis by other routes. For example, one alternative to produce methyl ethyl ketone and/or 2-butanol other than use of the methyl ethyl ketone and/or 2-butanol producers is through addition of another microbial organism capable of converting a methyl ethyl ketone and/or 2-butanol pathway intermediate to methyl ethyl ketone and/or 2-butanol. One such procedure includes, for example, the fermentation of a microbial organism that produces a methyl ethyl ketone and/or 2-butanol pathway intermediate. The methyl ethyl ketone and/or 2-butanol pathway intermediate can then be used as a substrate for a second microbial organism that converts the methyl ethyl ketone and/or 2-butanol pathway intermediate to methyl ethyl ketone and/or 2-butanol. The methyl ethyl ketone and/or 2-butanol pathway intermediate can be added directly to another culture of the second organism or the original culture of the methyl ethyl ketone and/or 2-butanol pathway intermediate producers can be depleted of these microbial organisms by, for example, cell separation, and then subsequent addition of the second organism to the fermentation broth can be utilized to produce the final product without intermediate purification steps.

In other embodiments, the non-naturally occurring microbial organisms and methods of the invention can be assembled in a wide variety of subpathways to achieve biosynthesis of, for example, methyl ethyl ketone and/or 2-butanol. In these embodiments, biosynthetic pathways for a desired product of the invention can be segregated into different microbial organisms, and the different microbial organisms can be co-cultured to produce the final product. In such a biosynthetic scheme, the product of one microbial organism is the substrate for a second microbial organism until the final product is synthesized. For example, the biosynthesis of methyl ethyl ketone and/or 2-butanol can be accomplished by constructing a microbial organism that contains biosynthetic pathways for conversion of one pathway intermediate to another pathway intermediate or the product. Alternatively, methyl ethyl ketone and/or 2-butanol also can be biosynthetically produced from microbial organisms through co-culture or co-fermentation using two organisms in the same vessel, where the first microbial organism produces a beta-ketovalerate, 2-methylacetoacetate, or methyl ethyl ketone (in the case of 2-butanol synthesis) intermediate and the second microbial organism converts the intermediate to methyl ethyl ketone and/or 2-butanol.

Given the teachings and guidance provided herein, those skilled in the art will understand that a wide variety of combinations and permutations exist for the non-naturally occurring microbial organisms and methods of the invention together with other microbial organisms, with the co-culture of other non-naturally occurring microbial organisms having subpathways and with combinations of other chemical and/or biochemical procedures well known in the art to produce methyl ethyl ketone and/or 2-butanol.

Sources of encoding nucleic acids for a methyl ethyl ketone and/or 2-butanol pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, S. cerevisiae, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite methyl ethyl ketone and/or 2-butanol biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of methyl ethyl ketone and/or 2-butanol described herein with reference to a particular organism such as S. cerevisiae can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative methyl ethyl ketone and/or 2-butanol biosynthetic pathway exists in an unrelated species, methyl ethyl ketone and/or 2-butanol biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods of the invention can be applied to all microbial organisms using the cognate metabolic alterations to those exemplified herein to construct a microbial organism in a species of interest that will synthesize methyl ethyl ketone and/or 2-butanol.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

Methods for constructing and testing the expression levels of a non-naturally occurring methyl ethyl ketone and/or 2-butanol -producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

Exogenous nucleic acid sequences involved in a pathway for production of methyl ethyl ketone and/or 2-butanol can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include one or more methyl ethyl ketone and/or 2-butanol biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors.

For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

Embodiments disclosed herein also provide a method for producing methyl ethyl ketone that includes culturing a non-naturally occurring microbial organism having a methyl ethyl ketone pathway. The pathway includes at least one exogenous nucleic acid encoding a methyl ethyl ketone pathway enzyme expressed in a sufficient amount to produce methyl ethyl ketone under conditions and for a sufficient period of time to produce methyl ethyl ketone. The methyl ethyl ketone pathway includes a β-ketothiolase, a β-ketovalerate decarboxylase and at least one of a β-ketovaleryl-CoA hydrolase and a β-ketovaleryl-CoA transferase.

Such cultured organisms also possess a propionyl-CoA pathway include at least one exogenous nucleic acid encoding a propionyl-CoA pathway enzyme expressed in a sufficient amount to produce propionyl-CoA described herein above, such as a PEP carboxylase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase. Additionally, as described above the cultured non-naturally occurring microbial organism also has acetyl-CoA pathway with at least one exogenous nucleic acid encoding an acetyl-CoA pathway enzyme expressed in a sufficient amount to produce acetyl-CoA. Such pathway includes one or more enzymes, such as a pyruvate kinase, a pyruvate formate lyase, and a formate hydrogen lyase.

In further embodiments, the present invention provides a method for producing 2-butanol that includes culturing a non-naturally occurring microbial organism having a 2-butanol pathway, said pathway comprising at least one exogenous nucleic acid encoding a 2-butanol pathway enzyme expressed in a sufficient amount to produce 2-butanol under conditions and for a sufficient period of time to produce 2-butanol, as described above, including having a β-ketothiolase, a β-ketovalerate decarboxylase, a methyl ethyl ketone reductase and an enzyme selected from the group consisting of a β-ketovaleryl-CoA hydrolase and a β-ketovaleryl-CoA transferase.

In still further embodiments, the present invention provides methods for producing methyl ethyl ketone and 2-butanol via culturing organisms having the alternate MEK pathway via 2-methylacetoacetate as described herein above.

In yet further embodiments, the present invention provides methods for producing methyl ethyl ketone or 2-butanol via culturing a non-naturally occurring microbial organism having the alternate propionyl-CoA pathway via threonine as described herein above. Thus, the methyl ethyl ketone pathway includes a propionyl-CoA pathway having a threonine deaminase. In some embodiments, the methyl ethyl ketone or 2-butanol pathways can include a β-ketothiolase, a β-ketovalerate decarboxylase, a methyl ethyl ketone reductase and an enzyme selected from a β-ketovaleryl-CoA hydrolase and a β-ketovaleryl-CoA transferase. While in other embodiments, the methyl ethyl ketone or 2-butanol pathways can include a 2-methylacetoacetyl-CoA thiolase, a 2-methylacetoacetate decarboxylase and an enzyme selected from a 2-methylacetoacetyl-CoA hydrolase and a 2-methylacetoacetyl-CoA transferase.

Suitable purification and/or assays to test for the production of methyl ethyl ketone and/or 2-butanol can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art. An assay for methylmalonyl-CoA mutase (MCM) has been reported (Birch et al. Journal of Bact., 175 (11), 1993) that measures dimethyl methylmalonate and dimethyl succinate after reaction of the crude protein extract of MCM in the presence of coenzyme B12 with methylmalonyl-CoA, followed by subsequent reaction with dimethyl ether. Multiple assays have been reported for β-ketothiolase (e.g., Slater et al., Journal of Bact., 180(8) (1998)). These assays rely on the change in the product concentrations as measured spectrophotometrically. A similar spectrophotometric assay for the succinyl-CoA:3-ketoacid-CoA transferase entails measuring the change in the absorbance corresponding to the product CoA molecule (i.e., succinyl-CoA) in the presence of the enzyme extract when supplied with succinate and β-ketoveleryl-CoA (Corthesy-Theulaz et al., Journal of Biological Chemistry, 272(41) (1997)). Succinyl-CoA can alternatively be measured in the presence of excess hydroxylamine by complexing the succinohydroxamic acid formed to ferric salts as referred to in (Corthesy-Theulaz et al., Journal of Biological Chemistry, 272(41) (1997)).

The methyl ethyl ketone and/or 2-butanol can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products of the invention. For example, the methyl ethyl ketone and/or 2-butanol producers can be cultured for the biosynthetic production of methyl ethyl ketone and/or 2-butanol.

For the production of methyl ethyl ketone and/or 2-butanol, the recombinant strains are cultured in a medium with carbon source and other essential nutrients. It is highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic conditions can be applied by perforating the septum with a small hole for limited aeration. Exemplary anaerobic conditions have been described previously and are well-known in the art. Exemplary aerobic and anaerobic conditions are described, for example, in U.S. patent application Ser. No. 11/891,602, filed Aug. 10, 2007. Fermentations can be performed in a batch, fed-batch or continuous manner, as disclosed herein.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring microorganism. Such sources include, for example, sugars such as glucose, xylose, arabinose, galactose, mannose, fructose and starch. Other sources of carbohydrate include, for example, renewable feedstocks and biomass. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms of the invention for the production of methyl ethyl ketone and/or 2-butanol.

In addition to renewable feedstocks such as those exemplified above, the methyl ethyl ketone and/or 2-butanol microbial organisms of the invention also can be modified for growth on syngas as its source of carbon. In this specific embodiment, one or more proteins or enzymes are expressed in the methyl ethyl ketone and/or 2-butanol producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

2 CO₂+4 H₂+n ADP+n Pi→CH₃COOH+2 H₂O+n ATP

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-THF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC). Following the teachings and guidance provided herein for introducing a sufficient number of encoding nucleic acids to generate a methyl ethyl ketone and/or 2-butanol pathway, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the microbial organisms of the invention such that the modified organism contains the complete Wood-Ljungdahl pathway will confer syngas utilization ability.

Accordingly, given the teachings and guidance provided herein, those skilled in the art will understand that a non-naturally occurring microbial organism can be produced that secretes the biosynthesized compounds of the invention when grown on a carbon source such as a carbohydrate.

Such compounds include, for example, methyl ethyl ketone and/or 2-butanol and any of the intermediate metabolites in the methyl ethyl ketone and/or 2-butanol pathway. All that is required is to engineer in one or more of the required enzyme or protein activities to achieve biosynthesis of the desired compound or intermediate including, for example, inclusion of some or all of the methyl ethyl ketone and/or 2-butanol biosynthetic pathways. Accordingly, the invention provides a non-naturally occurring microbial organism that produces and/or secretes methyl ethyl ketone and/or 2-butanol when grown on a carbohydrate or other carbon source and produces and/or secretes any of the intermediate metabolites shown in the methyl ethyl ketone and/or 2-butanol pathway when grown on a carbohydrate or other carbon source. The methyl ethyl ketone and/or 2-butanol producing microbial organisms of the invention can initiate synthesis from an intermediate, for example, beta-ketovalerate, 2-methylacetoacetate, or, in the case of 2-butanol synthesis, from MEK itself.

The non-naturally occurring microbial organisms of the invention are constructed using methods well known in the art as exemplified herein to exogenously express at least one nucleic acid encoding a methyl ethyl ketone and/or 2-butanol pathway enzyme or protein in sufficient amounts to produce methyl ethyl ketone and/or 2-butanol. It is understood that the microbial organisms of the invention are cultured under conditions sufficient to produce methyl ethyl ketone and/or 2-butanol. Following the teachings and guidance provided herein, the non-naturally occurring microbial organisms of the invention can achieve biosynthesis of methyl ethyl ketone and/or 2-butanol resulting in intracellular concentrations between about 0.1-2000 mM or more. Generally, the intracellular concentration of methyl ethyl ketone and/or 2-butanol is between about 3-2000 mM, particularly between about 50-1750 mM and more particularly between about 500-1500 mM, including about 600 mM, 900 mM, 1200 mM, 1500 mM, or more. Intracellular concentrations between and above each of these exemplary ranges also can be achieved from the non-naturally occurring microbial organisms of the invention.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. patent application Ser. No. 11/891,602, filed Aug. 10, 2007. Any of these conditions can be employed with the non-naturally occurring microbial organisms as well as other anaerobic conditions well known in the art. Under such anaerobic conditions, the methyl ethyl ketone and/or 2-butanol producers can synthesize methyl ethyl ketone and/or 2-butanol at intracellular concentrations of 5-10 mM or more as well as all other concentrations exemplified herein. It is understood that, even though the above description refers to intracellular concentrations, methyl ethyl ketone and/or 2-butanol producing microbial organisms can produce methyl ethyl ketone and/or 2-butanol intracellularly and/or secrete the product into the culture medium.

The culture conditions can include, for example, liquid culture procedures as well as fermentation and other large scale culture procedures. As described herein, particularly useful yields of the biosynthetic products of the invention can be obtained under anaerobic or substantially anaerobic culture conditions.

As described herein, one exemplary growth condition for achieving biosynthesis of methyl ethyl ketone and/or 2-butanol includes anaerobic culture or fermentation conditions. In certain embodiments, the non-naturally occurring microbial organisms of the invention can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of methyl ethyl ketone and/or 2-butanol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of methyl ethyl ketone and/or 2-butanol. Generally, and as with non-continuous culture procedures, the continuous and/or near-continuous production of methyl ethyl ketone and/or 2-butanol will include culturing a non-naturally occurring methyl ethyl ketone and/or 2-butanol producing organism of the invention in sufficient nutrients and medium to sustain and/or nearly sustain growth in an exponential phase. Continuous culture under such conditions can be include, for example, 1 day, 2, 3, 4, 5, 6 or 7 days or more. Additionally, continuous culture can include 1 week, 2, 3, 4 or 5 or more weeks and up to several months. Alternatively, organisms of the invention can be cultured for hours, if suitable for a particular application. It is to be understood that the continuous and/or near-continuous culture conditions also can include all time intervals in between these exemplary periods. It is further understood that the time of culturing the microbial organism of the invention is for a sufficient period of time to produce a sufficient amount of product for a desired purpose.

Fermentation procedures are well known in the art. Briefly, fermentation for the biosynthetic production of methyl ethyl ketone and/or 2-butanol can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the methyl ethyl ketone and/or 2-butanol producers of the invention for continuous production of substantial quantities of methyl ethyl ketone and/or 2-butanol, the methyl ethyl ketone and/or 2-butanol producers also can be, for example, simultaneously subjected to chemical synthesis procedures to convert the product to other compounds or the product can be separated from the fermentation culture and sequentially subjected to chemical conversion to convert the product to other compounds, if desired.

To generate better producers, metabolic modeling can be utilized to optimize growth conditions. Modeling can also be used to design gene knockouts that additionally optimize utilization of the pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on cell growth of shifting the metabolism towards more efficient production of methyl ethyl ketone and/or 2-butanol.

One computational method for identifying and designing metabolic alterations favoring biosynthesis of a desired product is the OptKnock computational framework (Burgard et al., Biotechnol. Bioeng. 84:647-657 (2003)). OptKnock is a metabolic modeling and simulation program that suggests gene disruption strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to either identify alternative pathways that lead to biosynthesis of a desired product or used in connection with the non-naturally occurring microbial organisms for further optimization of biosynthesis of a desired product.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that enable an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. patent application Ser. No. 11/891,602, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement biosynthesis of a desired compound in host microbial organisms. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The methods described above will provide one set of metabolic reactions to disrupt. Elimination of each reaction within the set or metabolic modification can result in a desired product as an obligatory product during the growth phase of the organism. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve production of a desired product are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the coupling of a product are desired or when genetic reversion is less likely to occur.

To identify additional productive solutions to the above described bilevel OptKnock problem which lead to further sets of reactions to disrupt or metabolic modifications that can result in the biosynthesis, including growth-coupled biosynthesis of a desired product, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatorily couples product biosynthesis to growth. For example, if a previously identified growth-coupled metabolic modification specifies reactions 1, 2, and 3 for disruption, then the following constraint prevents the same reactions from being simultaneously considered in subsequent solutions. The integer cut method is well known in the art and can be found described in, for example, Burgard et al., Biotechnol. Prog. 17:791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

The methods exemplified herein allow the construction of cells and organisms that biosynthetically produce a desired product, including the obligatory coupling of production of a target biochemical product to growth of the cell or organism engineered to harbor the identified genetic alterations. Therefore, the computational methods described herein allow the identification and implementation of metabolic modifications that are identified by an in silico method selected from OptKnock or SimPheny®. The set of metabolic modifications can include, for example, addition of one or more biosynthetic pathway enzymes and/or functional disruption of one or more metabolic reactions including, for example, disruption by gene deletion.

As discussed above, the OptKnock methodology was developed on the premise that mutant microbial networks can be evolved towards their computationally predicted maximum-growth phenotypes when subjected to long periods of growth selection. In other words, the approach leverages an organism's ability to self-optimize under selective pressures. The OptKnock framework allows for the exhaustive enumeration of gene deletion combinations that force a coupling between biochemical production and cell growth based on network stoichiometry. The identification of optimal gene/reaction knockouts requires the solution of a bilevel optimization problem that chooses the set of active reactions such that an optimal growth solution for the resulting network overproduces the biochemical of interest (Burgard et al., Biotechnol. Bioeng. 84:647-657 (2003)).

An in silico stoichiometric model of E. coli metabolism can be employed to identify essential genes for metabolic pathways as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. As disclosed herein, the OptKnock mathematical framework can be applied to pinpoint gene deletions leading to the growth-coupled production of a desired product. Further, the solution of the bilevel OptKnock problem provides only one set of deletions. To enumerate all meaningful solutions, that is, all sets of knockouts leading to growth-coupled production formation, an optimization technique, termed integer cuts, can be implemented. This entails iteratively solving the OptKnock problem with the incorporation of an additional constraint referred to as an integer cut at each iteration, as discussed above.

It is understood that modifications which do not substantially affect the activity of the various embodiments of this invention are also included within the definition of the invention provided herein. Accordingly, the following examples are intended to illustrate but not limit the present invention.

### Example I

### MEK Production in S. cerevisiae

This Example shows the insertion of genes into S. cerevisiae for the production of MEK.

Genes can be inserted into and expressed in S. cerevisiae using several methods. Some methods are plasmid-based whereas others allow for the incorporation of the gene into the chromosome (Guthrie and Fink. Guide to Yeast Genetics and Molecular and Cell Biology, Part B, Volume 350, Academic Press (2002); Guthrie and Fink, Guide to Yeast Genetics and Molecular and Cell Biology, Part C, Volume 351, Academic Press (2002)). High copy number plasmids using auxotrophic (e.g., URA3, TRP1, HIS3, LEU2) or antibiotic selectable markers (e.g., Zeo^{R} or Kan^{R}) can be used, often with strong, constitutive promoters such as PGK1 or ACT1 and a transcription terminator-polyadenylation region such as those from CYC1 or AOX. Many examples are available for one well-versed in the art. These include pVV214 (a 2 micron plasmid with URA3 selectable marker) and pVV200 (2 micron plasmid with TRP1 selectable marker) (Van et al., Yeast 20:739-746 (2003)). Alternatively, relatively low copy plasmids can be used. Again, many examples are available for one well-versed in the art. These include pRS313 and pRS315 (Sikorski and Hieter, Genetics 122:19-27 (1989) both of which require that a promoter (e.g., PGK1 or ACT1) and a terminator (e.g., CYC1, AOX) are added.

The integration of genes into the chromosome requires an integrative promoter-based expression vector, for example, a construct that includes a promoter, the gene of interest, a terminator, and a selectable marker with a promoter, flanked by FRT sites, loxP sites, or direct repeats enabling the removal and recycling of the resistance marker. The method entails the synthesis and amplification of the gene of interest with suitable primers, followed by the digestion of the gene at a unique restriction site, such as that created by the EcoRI and XhoI enzymes (Vellanki et al., Biotechnol Lett. 29:313-318 (2007)). The gene of interest is inserted at the EcoRI and XhoI sites into a suitable expression vector, downstream of the promoter. The gene insertion is verified by PCR and DNA sequence analysis. The recombinant plasmid is then linearized and integrated at a desired site into the chromosomal DNA of S. cerevisiae using an appropriate transformation method. The cells are plated on the YPD medium with the appropriate selection marker (e.g., kanamycin) and incubated for 2-3 days. The transformants are analyzed for the requisite gene insert by colony PCR.

To remove the antibiotic marker from a construct flanked by loxP sites, a plasmid containing the Cre recombinase is introduced. Cre recombinase promotes the excision of sequences flanked by loxP sites. (Gueldener et al., Nucleic Acids Res. 30:e23 (2002)). The resulting strain is cured of the Cre plasmid by successive culturing on media without any antibiotic present. The final strain has a markerless gene deletion, and thus the same method can be used to introduce multiple insertions in the same strain. Alternatively, the FLP-FRT system can be used in an analogous manner. This system involves the recombination of sequences between short Flipase Recognition Target (FRT) sites by the Flipase recombination enzyme (FLP) derived from the 2μ plasmid of the yeast Saccharomyces cerevisiae (Sadowski, P. D., Prog. Nucleic. Acid. Res. Mol. Biol. 51:53-91 (1995); Zhu and Sadowski J. Biol. Chem. 270:23044-23054 (1995)). Similarly, gene deletion methodologies will be carried out as described in refs. Baudin et al. Nucleic. Acids Res. 21:3329-3330 (1993); Brachmann et al., Yeast 14:115-132 (1998); Giaever et al., Nature 418:387-391 (2002); Longtine et al., Yeast 14:953-961 (1998) Winzeler et al., Science 285:901-906 (1999).

The engineered strains are characterized by measuring the growth rate, the substrate uptake rate, and the product/byproduct secretion rate. Cultures are grown overnight and used as inoculum for a fresh batch culture for which measurements are taken during exponential growth. The growth rate is determined by measuring optical density using a spectrophotometer (A600). Concentrations of glucose, MEK, alcohols, and other organic acid byproducts in the culture supernatant are determined by analytical methods including HPLC using an HPX-87H column (BioRad), or GC-MS, and used to calculate uptake and secretion rates. Cultures will be brought to steady state exponential growth via sub-culturing for enzyme assays. All experiments are performed with triplicate cultures.

### Example II

### MEK Production in E. coli and S. cerevisiae

This working Example shows the production of MEK in both engineered E. coli and S. cerevisiae as well as the organisms' tolerance to the MEK product.

The E. coli strain used was AB2 (AackA-pta, ApykA, ApykF, AdhaKLM) and the Yeast strains were BY4741 (his3Δ leu2Δ met15Δ ura3Δ) and ESY1 (BY4741 with pdc1Δ::kan and trp1Δ). Strain construction: Saccharomyces cerevisiae haploid strain BY4741 (MATa his3Δ1 leu2Δ0 met15Δ0 ura3Δ0) with pdc5 replaced with the Kanamycin resistance gene, pdc5::kanr (clone ID 4091) from the Saccharomyces Genome Deletion Project was further manipulated by a double crossover event using homologous recombination to replace the TRP1 gene with URA3. The resulting strain was grown on 5-FOA plates to “URA blast” the strain, thereby selecting for clones that had ura3 mutations. A clone from this plate was expanded and from then on dubbed “ESY1.” This strain with the final genotype BY4741 (MATa his3Δ1 leu2Δ0 met15Δ0 ura3Δ0 trp1::ura3 pdc5::kanr) was used for MEK heterologous pathway expression. Plasmid pUR400 (Schmid et al., J. Bacteriol. 151.1:68-76 (1982)) contains a PTS sucrose operon and was conjugated into AB2 for growth on sucrose. For bacterial pathway expressions, M9 medium was used; 1× M9 salts (6 g Na₂HPO₄, 3 g KH₂PO4, 0.5 g NaCl, 1 g NH₄Cl, dH₂O to approximately 1 liter (l)). Autoclave, when cooled, added 10 mL filter sterilized 100 mM MgSO₄, 10 mL sterile 20% glucose, 10 mM CaCl₂ before use. Additionally, 10 μg/ml Thiamine, 1× Trace Minerals, 10 uM B12 (cyano), 10 mM NaHCO₃ and 100 mM MOPS was added. For yeast gene expression, synthetic defined media which contains Yeast Nitrogen Base (1.7 g/L), ammonium sulfate (5 g/L) and a complete supplement mixture (CSM) of amino acids minus −His, −Leu, −Trp, −Ura, −dextrose was used (Sunrise Science Products, Inc. San Diego, Calif. catalog #1788-100). A carbon source either 0.2% glucose or 0.2% sucrose plus 2% galactose was added.

The genes used for cloning are shown below in Table 37.

TABLE 37

| | Species | | | | | |
|---|---|---|---|---|---|---|
| Enzyme | template | Gene | ORF SEQ | length | 5′ PRIMER | 3′ PRIMER |
| pyruvate | E. coli | pflB | atgtccgagcttaatgaaaagttagccacagcctggga | 2283 | ATGTCCGAG | TTACATAGAT |
| formate | | | aggttttaccaaaggtgactggcagaatgaagtaaacgt | | CTTAATGAA | TGAGTGAAGG |
| lyase | | | ccgtgacttcattcagaaaaactacactccgtacgaggg | | AAGTTAGCC | TACGAGTAAT |
| | | | tgacgagtccttcctggctggcgctactgaagcgacca | | | AACG |
| | | | ccaccctgtgggacaaagtaatggaaggcgttaaactg | | | |
| | | | gaaaaccgcactcacgcgccagttgactttgacaccgc | | | |
| | | | tgttgcttccaccatcacctctcacgacgctggctacatc | | | |
| | | | aacaagcagcttgagaaaatcgttggtctgcagactga | | | |
| | | | agctccgctgaaacgtgctcttatcccgttcggtggtatc | | | |
| | | | aaaatgatcgaaggttcctgcaaagcgtacaaccgcga | | | |
| | | | actggatccgatgatcaaaaaaatcttcactgaataccgt | | | |
| | | | aaaactcacaaccagggcgtgttcgacgtttacactccg | | | |
| | | | gacatcctgcgttgccgtaaatctggtgttctgaccggtc | | | |
| | | | tgccagatgcatatggccgtggccgtatcatcggtgact | | | |
| | | | accgtcgcgttgcgctgtacggtatcgactacctgatga | | | |
| | | | aagacaaactggcacagttcacttctctgcaggctgatc | | | |
| | | | tggaaaacggcgtaaacctggaacagactatccgtctg | | | |
| | | | cgcgaagaaatcgctgaacagcaccgcgctctgggtc | | | |
| | | | agatgaaagaaatggctgcgaaatacggctacgacatc | | | |
| | | | tctggtccggctaccaacgctcaggaagctatccagtg | | | |
| | | | gacttacttcggctacctggctgctgttaagtctcagaac | | | |
| | | | ggtgctgcaatgtccttcggtcgtacctccaccttcctgg | | | |
| | | | atgtgtacatcgaacgtgacctgaaagctggcaagatc | | | |
| | | | accgaacaagaagcgcaggaaatggttgaccacctgg | | | |
| | | | tcatgaaactgcgtatggttcgcttcctgcgtactccgga | | | |
| | | | atacgatgaactgttctctggcgacccgatctgggcaac | | | |
| | | | cgaatctatcggtggta | | | |
| pyruvate | E. coli | pflA | atgtcagttattggtcgcattcactcctttgaatcctgtgg | 741 | ATGTCAGTT | TTAGAACATT |
| formate | | | aaccgtagacggcccaggtattcgctttatcacctttttcc | | ATTGGTCGC | ACCTTATGAC |
| lyase | | | agggctgcctgatgcgctgcctgtattgtcataaccgcg | | ATTCACTC | CGTACTGCTC |
| activat- | | | acacctgggacacgcatggcggtaaagaagttaccgtt | | | |
| ing | | | gaagatttgatgaaggaagtggtgacctatcgccacttt | | | |
| enzyme | | | atgaacgcttccggcggcggcgttaccgcatccggcg | | | |
| | | | gtgaagcaatcctgcaagctgagtttgttcgtgactggtt | | | |
| | | | ccgcgcctgcaaaaaagaaggcattcatacctgtctgg | | | |
| | | | acaccaacggttttgttcgtcgttacgatccggtgattgat | | | |
| | | | gaactgctggaagtaaccgacctggtaatgctcgatctc | | | |
| | | | aaacagatgaacgacgagatccaccaaaatctggttgg | | | |
| | | | agtttccaaccaccgcacgctggagttcgctaaatatct | | | |
| | | | ggcgaacaaaaatgtgaaggtgtggatccgctacgttg | | | |
| | | | ttgtcccaggctggtctgacgatgacgattcagcgcatc | | | |
| | | | gcctcggtgaatttacccgtgatatgggcaacgttgaga | | | |
| | | | aaatcgagcttctcccctaccacgagctgggcaaacac | | | |
| | | | aaatgggtggcaatgggtgaagagtacaaactcgacg | | | |
| | | | gtgttaaaccaccgaagaaagagaccatggaacgcgt | | | |
| | | | gaaaggcattcttgagcagtacggtcataaggtaatgtt | | | |
| | | | ctaa | | | |
| pyruvate | E. coli | tdcE | atgaaggtagatattgataccagcgataagctgtacgcc | 2295 | ATGAAGGTA | ttAGAGCGCCT |
| formate | | | gacgcatggcttggctttaaaggtacggactggaaaaa | | GATATTGAT | GGGTAAAGGT |
| lyase 4; | | | cgaaattaatgtccgcgattttattcaacataactatacac | | ACCAGCGAT | ACG |
| 2-ketobu- | | | cgtatgaaggcgatgaatctttcctcgccgaagcgacg | | AAGC | |
| tyrate | | | cctgccaccacggaattgtgggaaaaagtaatggaag | | | |
| formate- | | | gcatccgtatcgaaaatgcaacccacgcgccggttgatt | | | |
| lyase | | | tcgataccaatattgccaccacaattaccgctcatgatgc | | | |
| | | | gggatatattaaccagccgctggaaaaaattgttggcct | | | |
| | | | gcaaacggatgcgccgttgaaacgtgcgctacacccgt | | | |
| | | | tcggtggcattaatatgattaaaagttcattccacgcctat | | | |
| | | | ggccgagaaatggacagtgaatttgaatatctgtttacc | | | |
| | | | gatctgcgtaaaacccataaccagggcgtatttgatgttt | | | |
| | | | actcaccggatatgctgcgctgccgtaaatctggcgtgc | | | |
| | | | tgaccggtttaccagatggctatggccgtgggcgcatta | | | |
| | | | tcggtgactatcgccgcgtagcgctgtatggcatcagtt | | | |
| | | | atctggtacgtgaacgcgaactgcaatttgccgatctcc | | | |
| | | | agtctcgtctggaaaaaggcgaggatctggaagccac | | | |
| | | | catccgtctgcgtgaggagctggcagagcatcgtcatg | | | |
| | | | cgctgttgcagattcaggaaatggcggcgaaatatggc | | | |
| | | | tttgatatctctcgcccggcgcagaatgcgcaggaagc | | | |
| | | | ggtgcagtggctctacttcgcttatctggcggcagtgaa | | | |
| | | | atcgcaaaatggcggcgcgatgtcgctgggccgcacg | | | |
| | | | gcatcgttcctcgatatctacattgagcgcgactttaaag | | | |
| | | | ctggcgtactcaatgagcagcaggcacaggaactgatc | | | |
| | | | gatcacttcatcatgaagatccgtatggtacgcttcctgc | | | |
| | | | gtacaccggaatttgattcgctgttctccggcgacccaat | | | |
| | | | ctgggcgacggaag | | | |
| stress- | E. coli | yfiD | atgattacaggtatccagattactaaagccgctaacgac | 384 | ATGATTACA | TTACAGGCTT |
| induced | | | gatctgctgaactctttctggctgctggacagcgaaaaa | | GGTATCCAG | TCAGTAAAGG |
| alternate | | | ggcgaagcgcgttgcatcgttgcaaaagcaggttatgc | | ATTACTAAA | TACGAGC |
| pyruvate | | | agaagatgaagtggttgcagtaagcaaactgggtgaca | | GCCG | |
| formate- | | | ttgaataccgtgaagttccagtagaagtgaaaccagaa | | | |
| lyase | | | gttcgcgttgaaggtggtcaacacctgaacgttaacgtt | | | |
| subunit | | | ctgcgtcgcgaaactctggaagatgcagttaagcatcc | | | |
| | | | ggaaaaatatccgcagctgaccatccgtgtatccggtta | | | |
| | | | tgcagttcgctttaactctctgactccggaacagcagcg | | | |
| | | | cgacgttatcgctcgtacctttactgaaagcctgtaa | | | |
| methyl- | E. coli | sbm | atgtctaacgtgcaggagtggcaacagcttgccaacaa | 2145 | ATGTCTAAC | TTAATCATGA |
| malonyl- | | | ggaattgagccgtcgggagaaaactgtcgactcgctgg | | GTGCAGGAG | TGCTGGCTTA |
| CoA | | | ttcatcaaaccgcggaagggatcgccatcaagccgctg | | TGGCAAC | TCAGATTCAG |
| mutase | | | tataccgaagccgatctcgataatctggaggtgacaggt | | | |
| (scpA) | | | acccttcctggtttgccgccctacgttcgtggcccgcgt | | | |
| | | | gccactatgtataccgcccaaccgtggaccatccgtca | | | |
| | | | gtatgctggtttttcaacagcaaaagagtccaacgcttttt | | | |
| | | | atcgccgtaacctggccgccgggcaaaaaggtctttcc | | | |
| | | | gttgcgtttgaccttgccacccaccgtggctacgactcc | | | |
| | | | gataacccgcgcgtggcgggcgacgtcggcaaagcg | | | |
| | | | ggcgtcgctatcgacaccgtggaagatatgaaagtcct | | | |
| | | | gttcgaccagatcccgctggataaaatgtcggtttcgat | | | |
| | | | gaccatgaatggcgcagtgctaccagtactggcgtttta | | | |
| | | | tatcgtcgccgcagaagagcaaggtgttacacctgata | | | |
| | | | aactgaccggcaccattcaaaacgatattctcaaagagt | | | |
| | | | acctctgccgcaacacctatatttacccaccaaaaccgt | | | |
| | | | caatgcgcattatcgccgacatcatcgcctggtgttccg | | | |
| | | | gcaacatgccgcgatttaataccatcagtatcagcggtt | | | |
| | | | accacatgggtgaagcgggtgccaactgcgtgcagca | | | |
| | | | ggtagcatttacgctcgctgatgggattgagtacatcaa | | | |
| | | | agcagcaatctctgccggactgaaaattgatgacttcgc | | | |
| | | | tcctcgcctgtcgttcttcttcggcatcggcatggatctgt | | | |
| | | | ttatgaacgtcgccatgttgcgtgcggcacgttatttatg | | | |
| | | | gagcgaagcggtcagtggatttggcgcacaggacccg | | | |
| | | | aaatcactggcgctgcgtacccactgccagacctcagg | | | |
| | | | ctggagcctgactgaacaggatccgtataacaacgttat | | | |
| | | | ccgcaccaccattgaagcgc | | | |
| arginine | E. coli | ygfD | atgattaatgaagccacgctggcagaaagtattcgccg | 996 | n/a | n/a |
| transport | | | cttacgtcagggtgagcgtgccacactcgcccaggcca | | | |
| ATPase | | | tgacgctggtggaaagccgtcacccgcgtcatcaggc | | | |
| (argK) | | | actaagtacgcagctgcttgatgccattatgccgtactgc | | | |
| | | | ggtaacaccctgcgactgggcgttaccggcacccccg | | | |
| | | | gcgcggggaaaagtacctttcttgaggcctttggcatgtt | | | |
| | | | gttgattcgagagggattaaaggtcgcggttattgcggt | | | |
| | | | cgatcccagcagcccggtcactggcggtagcattctcg | | | |
| | | | gggataaaacccgcatgaatgacctggcgcgtgccga | | | |
| | | | agcggcgtttattcgcccggtaccatcctccggtcatctg | | | |
| | | | ggcggtgccagtcagcgagcgcgggaattaatgctgtt | | | |
| | | | atgcgaagcagcgggttatgacgtagtgattgtcgaaa | | | |
| | | | cggttggcgtcgggcagtcggaaacagaagtcgcccg | | | |
| | | | catggtggactgttttatctcgttgcaaattgccggtggc | | | |
| | | | ggcgatgatctgcagggcattaaaaaagggctgatgga | | | |
| | | | agtggctgatctgatcgttatcaacaaagacgatggcga | | | |
| | | | taaccataccaatgtcgccattgcccggcatatgtacga | | | |
| | | | gagtgccctgcatattctgcgacgtaaatacgacgaatg | | | |
| | | | gcagccacgggttctgacttgtagcgcactggaaaaac | | | |
| | | | gtggaatcgatgagatctggcacgccatcatcgacttca | | | |
| | | | aaaccgcgctaactgccagtggtcgtttacaacaagtgc | | | |
| | | | ggcaacaacaatcggtggaatggctgcgtaagcagac | | | |
| | | | cgaagaagaagtactgaatcacctgttcgcgaatgaag | | | |
| | | | atttcgatcgctattaccgccagacgcttttagcggtcaa | | | |
| | | | aaacaatacgctctcaccgcgcaccggcctgcggcag | | | |
| | | | ctcagtgaatttatccagacgcaatattttgattaa | | | |
| methyl- | E. coli | ygfG | atgtcttatcagtatgttaacgttgtcactatcaacaaagt | 786 | ATGTCTTATC | TTAATGACCA |
| malonyl- | | | ggcggtcattgagtttaactatggccgaaaacttaatgcc | | AGTATGTTA | ACGAAATTAG |
| CoA de- | | | ttaagtaaagtctttattgatgatcttatgcaggcgttaagc | | ACGTTGTCA | GTTTACG |
| carboxyl- | | | gatctcaaccggccggaaattcgctgtatcattttgcgcg | | CTATC | |
| ase (scpB) | | | caccgagtggatccaaagtcttctccgcaggtcacgata | | | |
| | | | ttcacgaactgccgtctggcggtcgcgatccgctctcct | | | |
| | | | atgatgatccattgcgtcaaatcacccgcatgatccaaa | | | |
| | | | aattcccgaaaccgatcatttcgatggtggaaggtagtg | | | |
| | | | tttggggtggcgcatttgaaatgatcatgagttccgatct | | | |
| | | | gatcatcgccgccagtacctcaaccttctcaatgacgcc | | | |
| | | | tgtaaacctcggcgtcccgtataacctggtcggcattca | | | |
| | | | caacctgacccgcgacgcgggcttccacattgtcaaag | | | |
| | | | agctgatttttaccgcttcgccaatcaccgcccagcgcg | | | |
| | | | cgctggctgtcggcatcctcaaccatgttgtggaagtgg | | | |
| | | | aagaactggaagatttcaccttacaaatggcgcaccac | | | |
| | | | atctctgagaaagcgccgttagccattgccgttatcaaa | | | |
| | | | gaagagctgcgtgtactgggcgaagcacacaccatga | | | |
| | | | actccgatgaatttgaacgtattcaggggatgcgccgcg | | | |
| | | | cggtgtatgacagcgaagattaccaggaagggatgaa | | | |
| | | | cgctttcctcgaaaaacgtaaacctaatttcgttggtcatt | | | |
| | | | aa | | | |
| beta- | Ralstonia | bktB | atgacgcgtgaagtggtagtggtaagcggtgtccgtac | 1185 | ATGACGCGT | ttAGATACGCT |
| ketothio- | eutropha H16 | | cgcgatcgggacctttggcggcagcctgaaggatgtg | | GAAGTGGTA | CGAAGATGGC |
| lase | | | gcaccggcggagctgggcgcactggtggtgcgcgag | | GTGGTAAG | GG |
| | | | gcgctggcgcgcgcgcaggtgtcgggcgacgatgtc | | | |
| | | | ggccacgtggtattcggcaacgtgatccagaccgagc | | | |
| | | | cgcgcgacatgtatctgggccgcgtcgcggccgtcaa | | | |
| | | | cggcggggtgacgatcaacgcccccgcgctgaccgt | | | |
| | | | gaaccgcctgtgcggctcgggcctgcaggccattgtca | | | |
| | | | gcgccgcgcagaccatcctgctgggcgataccgacgt | | | |
| | | | cgccatcggcggcggcgcggaaagcatgagccgcgc | | | |
| | | | accgtacctggcgccggcagcgcgctggggcgcacg | | | |
| | | | catgggcgacgccggcctggtcgacatgatgctgggt | | | |
| | | | gcgctgcacgatcccttccatcgcatccacatgggcgt | | | |
| | | | gaccgccgagaatgtcgccaaggaatacgacatctcg | | | |
| | | | cgcgcgcagcaggacgaggccgcgctggaatcgcac | | | |
| | | | cgccgcgcttcggcagcgatcaaggccggctacttcaa | | | |
| | | | ggaccagatcgtcccggtggtgagcaagggccgcaa | | | |
| | | | gggcgacgtgaccttcgacaccgacgagcacgtgcgc | | | |
| | | | catgacgccaccatcgacgacatgaccaagctcaggc | | | |
| | | | cggtcttcgtcaaggaaaacggcacggtcacggccgg | | | |
| | | | caatgcctcgggcctgaacgacgccgccgccgcggtg | | | |
| | | | gtgatgatggagcgcgccgaagccgagcgccgcggc | | | |
| | | | ctgaagccgctggcccgcctggtgtcgtacggccatgc | | | |
| | | | cggcgtggacccgaaggccatgggcatcggcccggt | | | |
| | | | gccggcgacgaagatcgcgctggagcgcgccggcct | | | |
| | | | gcaggtgtcggacctggacgtgatcgaagccaacgaa | | | |
| | | | gcctttgccgcacaggcgtgcgccgtgaccaaggcgc | | | |
| | | | tcggtctggacccggccaaggttaacccga | | | |
| beta- | Acinetobacter | phaA | atgaaagatgttgtgattgttgcagcaaaacgtactgcg | 1179 | ATGAAAGAT | TTAGTCACGT |
| keto- | sp. Strain | | attggtagctttttaggtagtcttgcatctttatctgcacca | | GTTGTGATTG | TCAACTGCAA |
| thiolase | RA3849 | | cagttggggcaaacagcaattcgtgcagttttagacagc | | TTGCAGC | GTGCAAC |
| | | | gctaatgtaaaacctgaacaagttgatcaggtgattatgg | | | |
| | | | gcaacgtactcacgacaggcgtgggacaaaaccctgc | | | |
| | | | acgtcaggcagcaattgctgctggtattccagtacaagt | | | |
| | | | gcctgcatctacgctgaatgtcgtctgtggttcaggtttg | | | |
| | | | cgtgcggtacatttggcagcacaagccattcaatgcgat | | | |
| | | | gaagccgacattgtggtcgcaggtggtcaagaatctat | | | |
| | | | gtcacaaagtgcgcactatatgcagctgcgtaatgggc | | | |
| | | | aaaaaatgggtaatgcacaattggtggatagcatggtg | | | |
| | | | gctgatggtttaaccgatgcctataaccagtatcaaatgg | | | |
| | | | gtattaccgcagaaaatattgtagaaaaactgggtttaaa | | | |
| | | | ccgtgaagaacaagatcaacttgcattgacttcacaaca | | | |
| | | | acgtgctgcggcagctcaggcagctggcaagtttaaag | | | |
| | | | atgaaattgccgtagtcagcattccacaacgtaaaggtg | | | |
| | | | agcctgttgtatttgctgaagatgaatacattaaagccaat | | | |
| | | | accagccttgaaagcctcacaaaactacgcccagccttt | | | |
| | | | aaaaaagatggtagcgtaaccgcaggtaatgcttcagg | | | |
| | | | cattaatgatggtgcagcagcagtactgatgatgagtgc | | | |
| | | | ggacaaagcagcagaattaggtcttaagccattggcac | | | |
| | | | gtattaaaggctatgccatgtctggtattgagcctgaaatt | | | |
| | | | atggggcttggtcctgtcgatgcagtaaagaaaaccctc | | | |
| | | | aacaaagcaggctggagcttagatcaggttgatttgatt | | | |
| | | | gaagccaatgaagcatttgctgcacaggctttgggtgtt | | | |
| | | | gctaaagaattaggcttagacctggataaagtcaacgtc | | | |
| | | | aatggcg | | | |
| succinyl- | Helicobacter | scoA | atgaacaaggttataaccgatttagacaaagcattgagc | 699 | ATGAATAAG | ttATTTCGTGCT |
| CoA: 3- | pylori | | gggttaaaagacggggacactattttagtgggcggtttt | | GTCATAACC | CCTTGTGGTG |
| ketoacid | | | gggctgtgcgggatacccgaatacgccattaattacattt | | GATTTAGAC | ATTTTTTC |
| CoA | | | ataagaaaggcattaaggatttgattgtcgtgagcaataa | | AAAG | |
| trans- | | | ttgcggcgttgatgactttgggttgggcattcttttagaaa | | | |
| ferase A | | | aaaaacagattaaaaagattatcgcttcctatgtgggag | | | |
| | | | aaaataagatttttgaatcgcaaatgctgaacggagaaa | | | |
| | | | ttgaagtcgttttgacaccgcaaggcacgctagctgaaa | | | |
| | | | acttgcgcgctggaggggctgggatacccgcttactac | | | |
| | | | accccaaccggtgttgggactttgatcgctcaaggcaa | | | |
| | | | ggaatcaagggagtttaacggcaaagagtatattttaga | | | |
| | | | aagagcgatcacaggcgattacgggcttatcaaagcct | | | |
| | | | ataaaagcgatactttagggaatttggtgttcagaaagac | | | |
| | | | agccaggaatttcaatcccttgtgcgcgatggcggcaa | | | |
| | | | aaatatgcgtcgctgaagtggaagaaattgtcccggcc | | | |
| | | | ggggaattagacccagatgaaatacacttgccaggaat | | | |
| | | | ctatgtgcaacacatctataagggcgagaaatttgaaaa | | | |
| | | | acggatagaaagaatcactacaaggagcgcgaaatga | | | |
| succinyl- | Helicobacter | scoB | atgagagaggctatcattaaaagagcggcaaaggaatt | 624 | ATGAGAGAG | tTATAAGCGC |
| CoA: 3- | pylori | | aaaagagggcatgtatgtgaatttagggataggtttgcc | | GCTATCATTA | ACCTCAAATT |
| ketoacid | | | cacgctggtggctaatgaagtgagcgggatgaatatcg | | AAAGAGCGG | CAGCTTC |
| CoA | | | ttttccaaagcgagaacgggttattagggattggcgctta | | | |
| trans- | | | ccctttagaagggggcgttgatgcggatctcattaatgc | | | |
| ferase B | | | aggaaaggaaaccataaccgtggtgccgggcgcttcg | | | |
| | | | ttttttaatagcgcggattcgtttgcgatgattcgtggggg | | | |
| | | | gcatattgatttagcgattttaggagggatggaagtctca | | | |
| | | | caaaatggggatttggctaattggatgatccctaaaaag | | | |
| | | | ctcataaaaggcatgggaggggctatggatttggtgcat | | | |
| | | | ggcgctaaaaaagtgattgtcatcatggagcattgcaac | | | |
| | | | aaatacggggagtctaaagtgaaaaaagaatgctcatt | | | |
| | | | gcccttaacgggaaaaggcgtggtgcatcaattgataa | | | |
| | | | cggatttagcggtgtttgaattttccaataacgccatgaa | | | |
| | | | attagtggaattgcaagagggggtcagccttgatcaagt | | | |
| | | | gagagaaaaaacagaagccgaatttgaagtgcacctat | | | |
| | | | ag | | | |
| succinyl- | Bacillus | scoA | atgggaaaagtgctgtcatcaagcaaggaagctgcga | 717 | ATGGGAAAA | ttACTTGGCCT |
| CoA: 3- | subtilis | | aactgattcatgatggggatacgctgatcgcgggaggg | | GTGCTGTCAT | CACCCTTTCC |
| ketoacid | | | tttgggctgtgcggcatccctgaacagctcattttgtctat | | CAAGC | CG |
| CoA | | | aagagatcagggagtaaaggatttaaccgttgtcagca | | | |
| trans- | | | ataactgcggagtcgatgactgggggcttggtttgcttct | | | |
| ferase A | | | ggctaacaagcaaatcaagaaaatgatcgcttcctatgt | | | |
| | | | cggtgaaaataaaatttttgagcggcagtttttaagcgga | | | |
| | | | gagcttgaggtagagcttgttccccaaggaacgctcgct | | | |
| | | | gagagaattcgtgcaggcggtgcaggcataccgggat | | | |
| | | | tttatacggcgacaggcgtcggcacctccatagccgag | | | |
| | | | ggaaaagaacataaaacattcggcggccggacttatgt | | | |
| | | | gctggagcgaggcattaccggcgatgtggcgatcgtc | | | |
| | | | aaagcgtggaaagcggacaccatgggcaatttgattttt | | | |
| | | | aggaaaacggcgagaaatttcaatcccattgccgccat | | | |
| | | | ggcaggcaagatcacgattgccgaggcggaagaaatc | | | |
| | | | gtggaagcaggagagctcgatccagatcacatccatac | | | |
| | | | gccgggaatttacgtacagcatgtcgtgcttggcgcga | | | |
| | | | gccaagaaaaacggattgaaaaacgaacagttcagca | | | |
| | | | agcatcgggaaagggtgaggccaagtga | | | |
| succinyl- | Bacillus | scoB | gtgaaggaagcgagaaaacgaatggtcaaacgggct | 651 | TGAAGGAAG | TTAAGAATTG |
| CoA: 3- | subtilis | | gtacaagaaatcaaggacggcatgaatgtgaatctcgg | | CGAGAAAAC | AGTACAGACT |
| ketoacid | | | gattggaatgccgacgcttgtcgcaaatgagatacccg | | GAATGG | GGCTTACAGC |
| CoA | | | atggcgttcacgtcatgcttcagtcggaaaacggcttgc | | | |
| trans- | | | tcggaattggcccctatcctctggaaggaacggaagac | | | |
| ferase B | | | gcggatttgatcaatgcgggaaaggaaacgatcactga | | | |
| | | | agtgacaggcgcctcttattttgacagcgctgagtcattc | | | |
| | | | gcgatgataagaggcgggcatatcgatttagctattctc | | | |
| | | | ggcggaatggaggtttcggagcagggggatttggcca | | | |
| | | | attggatgatcccgggcaaaatggtaaaagggatgggc | | | |
| | | | ggcgccatggatctcgtcaacggggcgaaacgaatcg | | | |
| | | | ttgtcatcatggagcacgtcaataagcatggtgaatcaa | | | |
| | | | aggtgaaaaaaacatgctcccttccgctgacaggccag | | | |
| | | | aaagtcgtacacaggctgattacggatttggctgtatttg | | | |
| | | | attttgtgaacggccgcatgacactgacggagcttcagg | | | |
| | | | atggtgtcacaattgaagaggtttatgaaaaaacagaag | | | |
| | | | ctgatttcgctgtaagccagtctgtactcaattcttaa | | | |
| acetoace- | E. coli | atoA | atggatgcgaaacaacgtattgcgcgccgtgtggcgca | 651 | n/a | n/a |
| tyl-CoA: | MG1655 | | agagcttcgtgatggtgacatcgttaacttagggatcggt | | | |
| acetyl- | | | ttacccacaatggtcgccaattatttaccggagggtattc | | | |
| CoA | | | atatcactctgcaatcggaaaacggcttcctcggtttagg | | | |
| tran- | | | cccggtcacgacagcgcatccagatctggtgaacgctg | | | |
| ferase A | | | gcgggcaaccgtgcggtgttttacccggtgcagccatg | | | |
| | | | tttgatagcgccatgtcatttgcgctaatccgtggcggtc | | | |
| | | | atattgatgcctgcgtgctcggcggtttgcaagtagacg | | | |
| | | | aagaagcaaacctcgcgaactgggtagtgcctgggaa | | | |
| | | | aatggtgcccggtatgggtggcgcgatggatctggtga | | | |
| | | | ccgggtcgcgcaaagtgatcatcgccatggaacattgc | | | |
| | | | gccaaagatggttcagcaaaaattttgcgccgctgcacc | | | |
| | | | atgccactcactgcgcaacatgcggtgcatatgctggtt | | | |
| | | | actgaactggctgtctttcgttttattgacggcaaaatgtg | | | |
| | | | gctcaccgaaattgccgacgggtgtgatttagccaccgt | | | |
| | | | gcgtgccaaaacagaagctcggtttgaagtcgccgcc | | | |
| | | | gatctgaatacgcaacggggtgatttatga | | | |
| acetoace- | E. coli | atoD | atgaaaacaaaattgatgacattacaagacgccaccgg | 663 | n/a | n/a |
| tyl-CoA: | MG1655 | | cttctttcgtgacggcatgaccatcatggtgggcggattt | | | |
| acetyl- | | | atggggattggcactccatcccgcctggttgaagcatta | | | |
| CoA | | | ctggaatctggtgttcgcgacctgacattgatagccaat | | | |
| tran- | | | gataccgcgtttgttgataccggcatcggtccgctcatc | | | |
| ferase D | | | gtcaatggtcgagtccgcaaagtgattgcttcacatatcg | | | |
| | | | gcaccaacccggaaacaggtcggcgcatgatatctggt | | | |
| | | | gagatggacgtcgttctggtgccgcaaggtacgctaat | | | |
| | | | cgagcaaattcgctgtggtggagctggacttggtggtttt | | | |
| | | | ctcaccccaacgggtgtcggcaccgtcgtagaggaag | | | |
| | | | gcaaacagacactgacactcgacggtaaaacctggct | | | |
| | | | gctcgaacgcccactgcgcgccgacctggcgctaattc | | | |
| | | | gcgctcatcgttgcgacacacttggcaacctgacctatc | | | |
| | | | aacttagcgcccgcaactttaaccccctgatagcccttg | | | |
| | | | cggctgatatcacgctggtagagccagatgaactggtc | | | |
| | | | gaaaccggcgagctgcaacctgaccatattgtcacccc | | | |
| | | | tggtgccgttatcgaccacatcatcgtttcacaggagag | | | |
| | | | caaataa | | | |
| acetoace- | Clostridium | ctfA | atgaactctaaaataattagatttgaaaatttaaggtcattc | 657 | ATGAACTCT | TTATGCAGGC |
| tyl-CoA: | acetobutyli- | | tttaaagatgggatgacaattatgattggaggttttttaaa | | AAAATAATT | TCCTTTACTAT |
| acetyl- | cum ATCC 824 | | ctgtggcactccaaccaaattaattgattttttagttaattta | | AGATTTGAA | ATAATTTATA |
| CoA | | | aatataaagaatttaacgattataagtaatgatacatgttat | | AATTTAAGG | AGAAC |
| tran- | | | cctaatacaggtattggtaagttaatatcaaataatcaagt | | TC | |
| ferase A | | | aaaaaagcttattgcttcatatataggcagcaacccagat | | | |
| | | | actggcaaaaaactttttaataatgaacttgaagtagagc | | | |
| | | | tctctccccaaggaactctagtggaaagaatacgtgcag | | | |
| | | | gcggatctggcttaggtggtgtactaactaaaacaggttt | | | |
| | | | aggaactttgattgaaaaaggaaagaaaaaaatatctat | | | |
| | | | aaatggaacggaatatttgttagagctacctcttacagcc | | | |
| | | | gatgtagcattaattaaaggtagtattgtagatgaggccg | | | |
| | | | gaaacaccttctataaaggtactactaaaaactttaatcc | | | |
| | | | ctatatggcaatggcagctaaaaccgtaatagttgaagc | | | |
| | | | tgaaaatttagttagctgtgaaaaactagaaaaggaaaa | | | |
| | | | agcaatgacccccggagttcttataaattatatagtaaag | | | |
| | | | gagcctgcataa | | | |
| acetoace- | Clostridium | ctfB | atgattaatgataaaaacctagcgaaagaaataatagcc | 666 | ATGATTAAT | TTAAACAGCC |
| tyl-CoA: | acetobutyli- | | aaaagagttgcaagagaattaaaaaatggtcaacttgta | | GATAAAAAC | ATGGGTCTAA |
| acetyl- | cum ATCC 824 | | aacttaggtgtaggtcttcctaccatggttgcagattatat | | CTAGCGAAA | GTTCATTG |
| CoA | | | accaaaaaatttcaaaattactttccaatcagaaaacgga | | GAAATAATA | |
| tran- | | | atagttggaatgggcgctagtcctaaaataaatgaggca | | G | |
| ferase B | | | gataaagatgtagtaaatgcaggaggagactatacaac | | | |
| | | | agtacttcctgacggcacatttttcgatagctcagtttcgtt | | | |
| | | | ttcactaatccgtggtggtcacgtagatgttactgttttag | | | |
| | | | gggctctccaggtagatgaaaagggtaatatagccaatt | | | |
| | | | ggattgttcctggaaaaatgctctctggtatgggtggag | | | |
| | | | ctatggatttagtaaatggagctaagaaagtaataattgc | | | |
| | | | aatgagacatacaaataaaggtcaacctaaaattttaaaa | | | |
| | | | aaatgtacacttcccctcacggcaaagtctcaagcaaat | | | |
| | | | ctaattgtaacagaacttggagtaattgaggttattaatga | | | |
| | | | tggtttacttctcactgaaattaataaaaacacaaccattg | | | |
| | | | atgaaataaggtctttaactgctgcagatttactcatatcc | | | |
| | | | aatgaacttagacccatggctgtttag | | | |
| acetoace- | Clostridium | adc | atgttaaaggatgaagtaattaaacaaattagcacgccat | 735 | ATGTTAAAG | TTACTTAAGA |
| tate de- | acetobutyli- | | taacttcgcctgcatttcctagaggaccctataaatttcat | | GATGAAGTA | TAATCATATA |
| carboxyl- | cum ATCC 824 | | aatcgtgagtattttaacattgtatatcgtacagatatggat | | ATTAAACAA | TAACTTCAGC |
| ase | | | gcacttcgtaaagttgtgccagagcctttagaaattgatg | | ATTAGCAC | TCTAGGC |
| (735aa) | | | agcccttagtcaggtttgaaattatggcaatgcatgatac | | | |
| | | | gagtggacttggttgttatacagaaagcggacaggctat | | | |
| | | | tcccgtaagctttaatggagttaagggagattatcttcata | | | |
| | | | tgatgtatttagataatgagcctgcaattgcagtaggaag | | | |
| | | | ggaattaagtgcatatcctaaaaagctcgggtatccaaa | | | |
| | | | gctttttgtggattcagatactttagtaggaactttagacta | | | |
| | | | tggaaaacttagagttgcgacagctacaatggggtaca | | | |
| | | | aacataaagccttagatgctaatgaagcaaaggatcaa | | | |
| | | | atttgtcgccctaattatatgttgaaaataatacccaattat | | | |
| | | | gatggaagccctagaatatgtgagcttataaatgcgaaa | | | |
| | | | atcacagatgttaccgtacatgaagcttggacaggacca | | | |
| | | | actcgactgcagttatttgatcacgctatggcgccactta | | | |
| | | | atgatttgccagtaaaagagattgtttctagctctcacatt | | | |
| | | | cttgcagatataatattgcctagagctgaagttatatatga | | | |
| | | | ttatcttaagtaa | | | |
| acetoace- | Clostridium | adc | atgttagaaagtgaagtatctaaacaaattacaactccac | 741 | ATGTTAGAA | TTATTTTACTG |
| tate de- | beijerinckii | | ttgctgctccagcgtttcctagaggaccatataggtttca | | AGTGAAGTA | AAAGATAATC |
| carboxyl- | | | caatagagaatatctaaacattatttatcgaactgatttag | | TCTAAACAA | ATGTACAACC |
| ase | | | atgctcttcgaaaaatagtaccagagccacttgaattaga | | ATTACAACT | TTAGG |
| (741aa) | | | tagagcatatgttagatttgaaatgatggctatgcctgata | | C | |
| | | | caaccggactaggctcatatacagaatgtggtcaagcta | | | |
| | | | ttccagtaaaatataatggtgttaagggtgactacttgcat | | | |
| | | | atgatgtatctagataatgaacctgctattgctgttggaag | | | |
| | | | agaaagtagcgcttatccaaaaaagcttggctatccaaa | | | |
| | | | gctatttgttgattcagatactttagttgggacacttaaata | | | |
| | | | tggtacattaccagtagctactgcaacaatgggatataa | | | |
| | | | gcacgagcctctagatcttaaagaagcctatgctcaaatt | | | |
| | | | gcaagacccaattttatgctaaaaatcattcaaggttacg | | | |
| | | | atggtaagccaagaatttgtgaactaatatgtgcagaaa | | | |
| | | | atactgatataactattcacggtgcttggactggaagtgc | | | |
| | | | acgtctacaattatttagccatgcactagctcctcttgctg | | | |
| | | | atttacctgtattagagattgtatcagcatctcatatcctca | | | |
| | | | cagatttaactcttggaacacctaaggttgtacatgattat | | | |
| | | | ctttcagtaaaataa | | | |
| threonine | E. coli | tdcB | atgcatattacatacgatctgccggttgctattgatgacat | 990 | ATGCATATT | TTAAGCGTCA |
| deaminase | | | tattgaagcgaaacaacgactggctgggcgaatttataa | | ACATACGAT | ACGAAACCGG |
| | | | aacaggcatgcctcgctccaactattttagtgaacgttgc | | CTGCCGG | TG |
| | | | aaaggtgaaatattcctgaagtttgaaaatatgcagcgta | | | |
| | | | cgggttcatttaaaattcgtggcgcatttaataaattaagtt | | | |
| | | | cactgaccgatgcggaaaaacgcaaaggcgtggtggc | | | |
| | | | ctgttctgcgggcaaccatgcgcaaggggtttccctctc | | | |
| | | | ctgcgcgatgctgggtatcgacggtaaagtggtgatgc | | | |
| | | | caaaaggtgcgccaaaatccaaagtagcggcaacgtg | | | |
| | | | cgactactccgcagaagtcgttctgcatggtgataacttc | | | |
| | | | aacgacactatcgctaaagtgagcgaaattgtcgaaatg | | | |
| | | | gaaggccgtatttttatcccaccttacgatgatccgaaag | | | |
| | | | tgattgctggccagggaacgattggtctggaaattatgg | | | |
| | | | aagatctctatgatgtcgataacgtgattgtgccaattggt | | | |
| | | | ggtggcggtttaattgctggtattgcggtggcaattaaat | | | |
| | | | ctattaacccgaccattcgtgttattggcgtacagtctgaa | | | |
| | | | aacgttcacggcatggcggcttctttccactccggagaa | | | |
| | | | ataaccacgcaccgaactaccggcaccctggcggatg | | | |
| | | | gttgtgatgtctcccgcccgggtaatttaacttacgaaat | | | |
| | | | cgttcgtgaattagtcgatgacatcgtgctggtcagcga | | | |
| | | | agacgaaatcagaaacagtatgattgccttaattcagcg | | | |
| | | | caataaagtcgtcaccgaaggcgcaggcgctctggcat | | | |
| | | | gtgctgcattattaagcggtaaattagaccaatatattcaa | | | |
| | | | aacagaaaaaccgtcagtattatttccggcggcaatatc | | | |
| | | | gatctttctcgcgtctctcaaatcaccggtttcgttgacgc | | | |
| | | | ttaa | | | |
| threonine | E. coli | ilvA | atggctgactcgcaacccctgtccggtgctccggaagg | 1545 | ATGGCTGAC | tTAACCCGCCA |
| deaminase | MG1655 | | tgccgaatatttaagagcagtgctgcgcgcgccggttta | | TCGCAACCC | AAAAGAACCT |
| | | | cgaggcggcgcaggttacgccgctacaaaaaatggaa | | CTG | GAAC |
| | | | aaactgtcgtcgcgtcttgataacgtcattctggtgaagc | | | |
| | | | gcgaagatcgccagccagtgcacagctttaagctgcgc | | | |
| | | | ggcgcatacgccatgatggcgggcctgacggaagaa | | | |
| | | | cagaaagcgcacggcgtgatcactgcttctgcgggtaa | | | |
| | | | ccacgcgcagggcgtcgcgttttcttctgcgcggttagg | | | |
| | | | cgtgaaggccctgatcgttatgccaaccgccaccgccg | | | |
| | | | acatcaaagtcgacgcggtgcgcggcttcggcggcga | | | |
| | | | agtgctgctccacggcgcgaactttgatgaagcgaaag | | | |
| | | | ccaaagcgatcgaactgtcacagcagcaggggttcac | | | |
| | | | ctgggtgccgccgttcgaccatccgatggtgattgccg | | | |
| | | | ggcaaggcacgctggcgctggaactgctccagcagg | | | |
| | | | acgcccatctcgaccgcgtatttgtgccagtcggcggc | | | |
| | | | ggcggtctggctgctggcgtggcggtgctgatcaaaca | | | |
| | | | actgatgccgcaaatcaaagtgatcgccgtagaagcgg | | | |
| | | | aagactccgcctgcctgaaagcagcgctggatgcggg | | | |
| | | | tcatccggttgatctgccgcgcgtagggctatttgctgaa | | | |
| | | | ggcgtaggcgtaaaacgcatcggtgacgaaaccttcc | | | |
| | | | gtttatgccaggagtatctcgacgacatcatcaccgtcg | | | |
| | | | atagcgatgcgatctgtgcggcgatgaaggatttattcg | | | |
| | | | aagatgtgcgcgcggtggcggaaccctctggcgcgct | | | |
| | | | ggcgctggcgggaatgaaaaaatatatcgccctgcaca | | | |
| | | | acattcgcggcgaacggctggcgcatattctttccggtg | | | |
| | | | ccaacgtgaacttccacggcctgcgctacgtctcagaa | | | |
| | | | cgctgcgaactgggcgaacagcgtgaagcgttgttgg | | | |
| phospho- | E. coli | ppc | atgaacgaacaatattccgcattgcgtagtaatgtcagta | 2652 | ATGAACGAA | TTAGCCGGTA |
| enol- | MG1655 | | tgctcggcaaagtgctgggagaaaccatcaaggatgc | | CAATATTCC | TTACGCATAC |
| pyruvate | | | gttgggagaacacattcttgaacgcgtagaaactatccg | | GCATTGC | CTGC |
| caboxyl- | | | taagttgtcgaaatcttcacgcgctggcaatgatgctaac | | | |
| ase | | | cgccaggagttgctcaccaccttacaaaatttgtcgaac | | | |
| | | | gacgagctgctgcccgttgcgcgtgcgtttagtcagttc | | | |
| | | | ctgaacctggccaacaccgccgagcaataccacagca | | | |
| | | | tttcgccgaaaggcgaagctgccagcaacccggaagt | | | |
| | | | gatcgcccgcaccctgcgtaaactgaaaaaccagccg | | | |
| | | | gaactgagcgaagacaccatcaaaaaagcagtggaat | | | |
| | | | cgctgtcgctggaactggtcctcacggctcacccaacc | | | |
| | | | gaaattacccgtcgtacactgatccacaaaatggtggaa | | | |
| | | | gtgaacgcctgtttaaaacagctcgataacaaagatatc | | | |
| | | | gctgactacgaacacaaccagctgatgcgtcgcctgcg | | | |
| | | | ccagttgatcgcccagtcatggcataccgatgaaatccg | | | |
| | | | taagctgcgtccaagcccggtagatgaagccaaatgg | | | |
| | | | ggctttgccgtagtggaaaacagcctgtggcaaggcgt | | | |
| | | | accaaattacctgcgcgaactgaacgaacaactggaag | | | |
| | | | agaacctcggctacaaactgcccgtcgaatttgttccgg | | | |
| | | | tccgttttacttcgtggatgggcggcgaccgcgacggc | | | |
| | | | aacccgaacgtcactgccgatatcacccgccacgtcct | | | |
| | | | gctactcagccgctggaaagccaccgatttgttcctgaa | | | |
| | | | agatattcaggtgctggtttctgaactgtcgatggttgaa | | | |
| | | | gcgacccctgaactgctggcgctggttggcgaagaag | | | |
| | | | gtgccgcagaaccgtatcgctatctgatgaaaaacctgc | | | |
| | | | gttctcgcctgatggcgacacaggcatggctggaagcg | | | |
| | | | cgcctgaaaggcgaagaactgccaaaac | | | |
| pyruvate | Streptococcus | pfl | atggcaactgtcaaaactaacactgacgtttttgaaaaag | 2328 | ATGGCAACT | TTATTTGTTGT |
| formate | mutans UA159 | | cctgggaaggctttaaaggaactgactggaaagacag | | GTCAAAACT | TAACCAAGTC |
| lyase | | | agcaagcatttctcgctttgttcaagacaactacactccat | | AACACTGAC | TGTAGCTGC |
| | | | atgacggagacgaaagttttcttgccggccctactgaac | | G | |
| | | | gttcacttcacatcaaaaaagtcgtagaagaaactaaag | | | |
| | | | cgcattacgaagaaacacgttttccaatggatacacgtat | | | |
| | | | tacatctattgctgatatcccagcaggttatattgacaagg | | | |
| | | | aaaatgaattgatttttggtatccaaaacgatgaacttttta | | | |
| | | | agctgaacttcatgccaaaaggcggtattcgcatggctg | | | |
| | | | aaacagctttgaaagaacatggttatgaaccagaccctg | | | |
| | | | ccgttcatgaaatctttaccaaatatgcaacaaccgttaat | | | |
| | | | gatggtatctttcgtgcttacacttcaaacattcgccgtgc | | | |
| | | | acgtcatgcccacactgtaactggtctcccagatgcata | | | |
| | | | ctctcgcggacgtattattggagtttatgcccgtcttgctc | | | |
| | | | tctatggtgctgactacttgatgcaagaaaaagtgaacg | | | |
| | | | actggaactcaattgctgaaattgatgaagaatcaattcg | | | |
| | | | tcttcgtgaagaaatcaatcttcaatatcaggcacttggc | | | |
| | | | gaagtagtgcggttgggtgatctgtatggtcttgatgttc | | | |
| | | | gcaaacctgctatgaatgttaaagaagctatccaatgga | | | |
| | | | ttaatatcgcctttatggctgtctgccgcgttatcaatggt | | | |
| | | | gctgcaacttctcttggacgtgtcccaatcgttcttgatat | | | |
| | | | ctttgcagaacgtgaccttgctcgtggcactttcactgaa | | | |
| | | | tcagaaatccaagaattcgttgatgacttcgttatgaaact | | | |
| | | | tcgtacggttaaatttgcacgtactaaggcttatgacgaa | | | |
| | | | ctttactcaggtgacccaacatttattacgacttctatggct | | | |
| | | | ggtatgggagctgatggacgtc | | | |
| pyruvate | Streptococcus | pflA | atgatagaaaaagttgactacgaaaaagtaacaggactt | 792 | ATGATAGAA | TTAATGATTA |
| formate | mutans UA159 | | gttaattctacagaatcttttgggtctgtagacggacctgg | | AAAGTTGAC | ATCCTCTTTTT |
| lyase ac- | | | tatacgctttgttgtttttatgcaagggtgccaaatgcgttg | | TACGAAAAA | ATATTCTTCAT |
| tivating | | | tcaatattgccacaatcctgatacttgggcaatgaagaat | | GTAACAGG | ATGTTTCC |
| enzyme | | | gatagagcaacagaaaggactgcaggagatgtctttaa | | | |
| | | | agaagctttacgttttaaagatttttggggagatacagga | | | |
| | | | ggtattactgtttctggtggtgaagcaacgctccagatgg | | | |
| | | | attttttaattgccctcttttctttagcaaaagaaaagggaa | | | |
| | | | ttcatacgaccttggatacctgtgctctgacttttagaaac | | | |
| | | | acaccaaaatatcttgaaaaatatgaaaagttaatggctg | | | |
| | | | tcactgatttagtattgttagatattaaagagattaatcctg | | | |
| | | | accaacataaaattgtcactggtcatagcaataaaactat | | | |
| | | | tttagcttgtgcgcgttatttatctgatattggaaaacctgtt | | | |
| | | | tggattcgccatgtcttagtccctggtctgactgatcggg | | | |
| | | | atgaagacttaataaagttgggtgagtatgtcaaaacact | | | |
| | | | gaagaatgttcaacggtttgaaattcttccttatcatacaat | | | |
| | | | gggtgaattcaaatggcgtgaattagggattccttatcctt | | | |
| | | | tggaaggtgttaaaccgccaacaccagatcgtgtgcgc | | | |
| | | | aatgctaaaaagttaatgcatacggaaacatatgaagaa | | | |
| | | | tataaaaagaggattaatcattaa | | | |
| pyruvate | Haemophilus | pfl | atgactatgtcagaacttaatgaaatgcaaaaattggcgt | 2319 | ATGACTATG | TTACATTGAC |
| formate | influenzae Rd | | gggctggttttgctggtggcgattggcaagaaaatgtca | | TCAGAACTT | TCTGTGAAAG |
| lyase | KW20 | | atgtacgtgactttatccaaaaaaactataccccttatgaa | | AATGAAATG | TTCTAGTAAT |
| | | | ggcgatgactctttcttagcaggtccaaccgaagcaaca | | CAAAAATTG | TACG |
| | | | accaagctttgggaatctgtgatggaaggtattaaaattg | | | |
| | | | aaaaccgtactcacgcgccattagattttgatgaacatac | | | |
| | | | accatctaccattatctctcacgcacctggttacattaaca | | | |
| | | | aagatttagaaaaaatcgttggtcttcaaactgatgaacc | | | |
| | | | tttaaaacgtgccattatgccattcggtggtatcaaaatg | | | |
| | | | gtggaaggttcttgtaaagtttatggtcgtgaacttgatcc | | | |
| | | | aaaagtgaaaaaaatcttcactgaataccgtaaaacaca | | | |
| | | | taaccaaggtgtattcgatgtttacacgccagatattttac | | | |
| | | | gttgccgtaaatctggggtattaactggtcttccagatgct | | | |
| | | | tatggtcgtggtcgtatcatcggtgactaccgtcgtgtag | | | |
| | | | cactttatggtgtagatttcttaatgaaagataaatacgca | | | |
| | | | caattctcttctttacaaaaagatttagaagatggcgtaaa | | | |
| | | | tcttgaagcaacaattcgtttacgtgaagaaatcgcaga | | | |
| | | | acaacaccgtgcattaggtcaattaaaacaaatggcag | | | |
| | | | caagctatggttatgatatttctaacccagcaactaatgct | | | |
| | | | caagaagccattcaatggatgtactttgcttatcttgctgc | | | |
| | | | aataaaatcacaaaatggtgctgcaatgtcattcggtcgt | | | |
| | | | accgcaacctttattgacgtgtacatcgaacgtgatttaa | | | |
| | | | aagcaggaaaaattactgaaactgaagcgcaagaatta | | | |
| | | | gttgaccacttagttatgaaacttcgtatggttcgtttctta | | | |
| | | | cgtacacctgaatacgatcaattattctctggtgacccaa | | | |
| | | | tgtgggcaactgaaaccatcg | | | |
| pyruvate | Haemophilus | pflA | atgtcagttcttggacgaattcactcttttgaatcctgtgg | 741 | ATGTCAGTTC | CTAGAATTTT |
| formate | influenzae Rd | | cactgtagatgggccaggtattcgttttattttatttatgca | | TTGGACGAA | ACAGTGTGTC |
| lyase ac- | KW20 | | aggctgcttgatgcgctgcaaatattgccacaatcgtgat | | TTCACTCTTT | CATAACCTTC |
| tivating | | | acttgggatcttgaaggtggtaaagaaatcagtgtcgaa | | TG | TAGG |
| enzyme | | | gatttaatgaaagaagtcgtgacttatcgccattttatgaa | | | |
| | | | tgctactggcggtggtgtcacagcatctggtggcgagg | | | |
| | | | ctgtgttacaagcagagtttgtacgcgattggttccgtgc | | | |
| | | | ttgtaaagaggaagggattaatacttgcttagatacaaat | | | |
| | | | ggttttgtacgtcattatgatcatattattgatgaattattag | | | |
| | | | atgtaacagatcttgttttacttgatttaaaagaacttaatg | | | |
| | | | atcaagttcatcaaaatcttattggggtgccaaataaacg | | | |
| | | | tacccttgaatttgcaaaatatttgcaaaaacgtaatcaac | | | |
| | | | atacctggattcgttatgttgtggttcctggttatactgata | | | |
| | | | gcgatcacgatgtgcatttattaggtcagtttattgaaggt | | | |
| | | | atgaccaatattgaaaaagttgaacttcttccttatcatcg | | | |
| | | | attaggtgtgcataaatggaaaacccttgggttagattat | | | |
| | | | gagcttgaaaatgtattaccgccaactaaagaatccttag | | | |
| | | | aacatattaaaacaatcctagaaggttatggacacactgt | | | |
| | | | aaaattctag | | | |

For pathway construction in E. coli, genes for the ygf operon which included the methylmalonyl-CoA mutase and the methylmalonyl-CoA decarboxylase were cloned into pZA33S. The thiolases and pZE23S and the various succinyl-CoA transferases were cloned into pZS*13S. To construct the pathway in S. cerevisiae, genes were cloned into pESC vectors pESC-HIS, pESC-LEU, pESC-TRP, and pESC-URA (Stratagene, cat #217455). These are shuttle vectors that can replicate in either E. coli or S. cerevisiae. They have dual galactose (GAL1, GAL10) divergent promoters that are inhibited in the presence of dextrose (glucose) but provide inducible expression in the presence of galactose sugar. The 3-ketoacid decarboxylase and the thiolases were cloned into pESC-His; succinyl-CoA transferases were cloned into pESC-Leu, yfiD and threonine deaminases were cloned into pESC-Trp; pyruvate formate lyases subunits A and B were cloned into pESC-Ura; and Hom3 G452D and pdcl-8 were cloned into pESC-Zeo.

All enzyme assays were performed from cells which had first expressed the appropriate gene(s). Cells were spun down, and lysed in a bead beater with glass beads, cell debris removed by centrifugation to generate crude extracts. Substrate was added to cell extracts and assayed for activity. Thiolase activity was determined by adding acetyl-CoA and propionyl-CoA to extracts. If the reaction condensed either of the CoA components, free CoA-SH was released. The free CoA-SH then complexed with DTNB to form DTNB-CoA, which was detected by absorbance at 410 nm. To assay aceotacetate decarboxylase activity, acetoacetate was added to extracts which was decarboxylated to acetone and CO₂. Acetoacetate absorbs at 270 nm so decreasing absorbance at this wavelength indicates enzyme activity. Likewise, acetoacetate-CoA absorbs at 304 nm and its decrease is used to monitor β-ketoacyl-CoA transferase activity when acetoacetate-CoA and succinate is added to the appropriate extracts. To detect pyruvate formate lyase activity in yeast, cells, extracts and reagents were all prepared anaerobically as the enzyme is known to be inhibited by oxygen. Because the DTNB-CoA reaction is inhibited by reducing agents required for the preparation of anaerobic extracts, assaying for the release of CoA-SH with DNTB could not be performed. Therefore, the products of the reactions (Acetyl-CoA or Propionyl-CoA) were directly analyzed by mass spectrometry to measure the products when extracts were provided with pyruvate or 2-ketobutyrate. Finally, threonine deaminase was assayed using a coupled assay. First threonine was added to extracts and if there was activity, α-ketobutyrate would be produced. The α-ketobutyrate could then be assayed by reducing it with NADH and lactate dehydrogenase. Decrease of NADH was then assayed by fluorescence since NADH absorbs light with wavelength of 340 nm and radiates secondary (fluorescence) photons with a wavelength of 450 nm.

For E. coli, AB2 cells were transformed with various combinations of genes and selected for the appropriate antibiotic markers. Transformants were picked and grown in 1 ml LB with selection. Subsequently, 250 μl of culture was injected into anaerobic vials with 10 ml of M9 media and grown semi-anaerobically using 23 gauge needle to vent the caps of the bottles. Each culture was induced with 0.5 mM IPTG and sampled after 24 hrs. Yeast cultures were inoculated into synthetic defined media without His, Leu, Trp, Ura. To increase MEK production, 1 or 5 g propionate was added for some samples.

Samples from MEK production culture were collected by removing a majority of cells by centrifugation at 17,000 rpm for five minutes at room temperature in a microcentrifuge. Supernatants were filtered through a 0.22 μm filter to remove trace amounts of cells and used directly for analysis by GC-MS.

To examine MEK tolerance, cells were initially tested by growth in MEK. For E. coli, strain MG1655 was grown in LB medium overnight and diluted 1:20 into fresh LB medium with various percentages of MEK (g/100 ml). Cultures were grown anaerobically in tightly capped bottles to prevent MEK from evaporating and decreasing the concentration of MEK in the bottle. For evolutions, cells were serially diluted 1:100 each day in various concentrations of LB with and without 5 μg/ml Nitroguanidine. If cultures grew to OD600 0.4 or more, they were again diluted with fresh media containing the same or slightly higher (0.5%) MEK. Yeast strain BY4741 was grown in YPD medium (10 g Yeast Extract, 20 g Bacto-peptone, 860 ml distilled H2O, after autoclaving add 100 ml 20% sterile glucose) with 10 μg/ml ergosterol and 420 μg/ml Tween-80+various concentrations of MEK. For evolutions, cultures were diluted to a starting OD600 of 0.2 and grown in various concentrations of MEK. Growth was performed in bottles with thick butyl rubber caps under anaerobic conditions.

To construct the pathways for yeast and E. coli, several genes were identified, cloned, sequenced and expressed from expression vectors. Genes and accession numbers are shown in Table 38. All the genes were cloned for the yeast pathway but not all were cloned for the E. coli pathway. For example, the pyruvate formate lyase (PFL), PFL activating enzyme (PflB) and the YfiD proteins did not need to be cloned and expressed from extra-chromosomal vectors as these genes are native to the E. coli and are induced under anaerobic conditions. Additionally, the Thr deaminases were only needed for the yeast pathway.

TABLE 38

| | Pyruvate Formate | β-Ketovalerate | | Succinyl-CaA:3-ketoacid | | Protection |
|---|---|---|---|---|---|---|
| PFL activator | Lysate (PFL) | decarboxylase (3) | β-keto thiolase | CoA transferase | Thr deaminase | peptide for pflB |
| pflA | PflB NP_415423) | Adc (NP_149328.1) | PhaA (AAA99475) | ScoA (NP_391778) and ScoB | llvA | YfiD |
| (NP_415422) | Eshericia coli | from Clostridium | Acinetobacter sp. | (NP_391777) Bacillus | (AAC77492) | (NP_417074) |
| Eschericia coli | | acetobutylicum | strain RA 3849 | subtillis | Eschericia coli | Eschericia coli |
| | TdcE | Adc (AAQ12071) | BktB | HPAG1_0676 (YP_627417) | TdcB | |
| | (YP_026205) | from Clostridium | (YP_725948.1) | and HPAG1_0677 | (AAC76152) | |
| | Eschericia coli | beijerinckii | Ralstonia eutropha | (YP_627418) from | Eschericia coli | |
| | | | | Heliobacter pylori | | |
| PflA | PflB | | | AtoA (NP_416726.1) and | | |
| (AAX87236) | (AAX87237.1) | | | AtoD (NP_416725.1) | | |
| H. influenzae | H. influenzae | | | Eschericia coli | | |
| | | | | CtfA (NP_149326.1) and | | |
| | | | | CtfB (NP_149327.1) | | |
| | | | | Clostridium acetobutylicum | | |

To determine if the pathways were capable of producing MEK, gene combinations were cloned into appropriate E. coli expression vectors and then transformed into the strain AB2. This strain was engineered to overproduce succinate and would therefore help increase carbon flux to propionyl-CoA. As shown in Table 39, several gene combinations successfully produced MEK. In general, the phaA thiolase gene from Acinetobacter produced more MEK than btkB thiolase from Ralstonia eutropha. The β-ketovalerate decarboxylase from C. acetobutylicum worked better than the decarboxylase from C. beijerinckii especially when combined with the phaA gene. Finally, the succinyl-CoA transferase from H. pylori worked better than the transferase from B. subtilus except for the combination btkB from R. eutropha, adc from C. acetobutylicum and CoA transferase from B. subtilus. The combination of genes that produced the highest amount of MEK (1.92 mM) was phaA from Acinetobacter, decarboxylase from C. acetobutylicum and CoA transferase from H. pylori.

Cultures with the complete pathways all produced acetone in greater amounts than MEK. There was a strong correlation between the amount of acetone produced and the amount of MEK made; the best combination of genes for MEK production was also the best for producing acetone. The ratio of acetone:MEK ranged from 3:1 to 20:1.

Finally, MEK was produced from sucrose when AB2 cells contained the plasmid pUR400 which contains a PTS sucrose operon. The amount of acetone and MEK and were very similar to that grown in glucose with the same plasmid concentrations with the exception of the combination of phaA from Acinetobacter, decarboxylase from C. acetobutylicum and CoA transferase from H. pylori. While this produced the highest amount of MEK from glucose at 1.92 mM, it only produced 1.01 mM MEK when grown on sucrose.

TABLE 39

| | | | CoA | 0 hr | 24 hr | mM | | mM/OD | | Ratio |
|---|---|---|---|---|---|---|---|---|---|---|
| condition | thiolase | decarboxylase | transferase | OD600 | OD600 | Acetone | MEK | Acetone | MEK | MEK/Acetone |
| μ- | phaA | adc-Ca | atoAD-Ec | 0.33 | 0.44 | 0.77 | 0.13 | 1.74 | .029 | .017 |
| aerobic | | | scoAB-Bs | 0.44 | 0.30 | 3.12 | 0.69 | 10.46 | 2.31 | 0.22 |
| | | | ctfAB-Ca | 0.26 | 0.29 | 0.09 | 0 | 0.31 | 0.00 | 0.00 |
| | | | scoAB-Hp | 0.45 | 0.78 | 5.72 | 1.92 | 7.34 | 2.46 | 0.34 |
| | | acd-Cb | atoAD-Ec | 0.30 | 0.77 | 1.17 | 0.11 | 1.53 | 0.14 | 0.09 |
| | | | scoAB-Bs | 0.27 | 0.55 | 3.48 | 0.82 | 6.34 | 1.49 | 0.24 |
| | | | ctfAB-Ca | 0.31 | 0.69 | 0.09 | 0 | 0.13 | 0.00 | 0.00 |
| | | | scoAB-Hp | 0.29 | 0.78 | 4.21 | 1.06 | 5.41 | 1.36 | 0.25 |
| | empty | empty | empty | 0.33 | 0.65 | 0 | 0 | 0.00 | 0.00 | — |
| μ- | btkB | adc-Ca | atoAD-Ec | 0.47 | 0.87 | 0.97 | 0.04 | 1.11 | 0.05 | 0.04 |
| aerobic | | | scoAB-Bs | 0.38 | 0.62 | 2.57 | 0.29 | 4.14 | 0.47 | 0.11 |
| | | | ctfAB-Ca | 0.47 | 1.00 | 0.07 | 0.00 | 0.07 | 0.00 | 0.00 |
| | | | scoAB-Hp | 0.48 | 0.96 | 2.14 | 0.10 | 2.24 | 0.10 | 0.05 |
| | | acd-Cb | atoAD-Ec | 0.40 | 0.73 | 0.03 | 0.00 | 0.04 | 0.00 | 0.00 |
| | | | scoAB-Bs | 0.45 | 0.91 | 0.29 | 0.00 | 0.32 | 0.00 | 0.00 |
| | | | ctfAB-Ca | 0.46 | 0.92 | 0.00 | 0.00 | 0.00 | 0.00 | — |
| | | | scoAB-Hp | 0.45 | 0.87 | 0.30 | 0.00 | 0.35 | 0.00 | 0.00 |
| | empty | empty | empty | 0.45 | 0.58 | 0.00 | 0.00 | 0.00 | 0.00 | — |
| μ- | phaA | acc-CA | scoAB-Bs | | 0.63 | 3.58 | 0.54 | 5.65 | 0.85 | 0.15 |
| aerobic, | | | scoAB-Hp | | 0.77 | 5.82 | 1.01 | 7.61 | 1.32 | 0.17 |
| sucrose | | adc-Cb | scoAB-Bs | | 0.58 | 3.48 | 0.68 | 5.98 | 1.17 | 0.20 |
| | | | scoAB-Hp | | 0.73 | 5.71 | 1.00 | 7.84 | 1.37 | 0.18 |
| | empty | empty | empty | | 0.63 | 0.02 | 0.00 | 0.03 | 0.00 | 0.00 |

For MEK production in S. cerevisiae, MEK yield was significantly less than for E. coli (Table 40). With no pathway genes, no acetone or MEK was produced, whereas when the pathway was present, acetone was formed. Many gene combinations were tried, but the PhaA thiolase and TdbC threonine deaminase were found to make the most detectable amounts of MEK (data not shown). When grown in standard medium, the best CoA transferase for making MEK appears to be CtfBA from C. acetobutylicum and the pyruvate formate lyase PflBA from H. influenza. The concentration of MEK is detectable by GC-MS but very low at approximately 0.3-0.5 μM. The exact concentration of MEK is difficult to quantify with certainty at these low levels. For acetone production, more is produced when using CoA transferase ScoAB from B. subtilis. The source of the pyruvate formate lyase does not appear to make much difference.

Another byproduct from this pathway is 1-propanol. Because of the observation that more 1-propanol is being made with cells expressing the pathway than empty vectors or ilvA (data not shown), it was determined that some of the 2-oxobutyrate made from the deamination of threonine might be diverted to 1-propanol. To reduce the amount of 1-propanol formation and increase MEK formation, 1 g or 5 g/L propionate was added to the medium. As seen in Table 40, propionate did have a favorable effect on MEK production increasing the levels of MEK from virtually unmeasurable to 4 to 8 μM. Less propionate appears to work better than more, but this may be due to toxic effects of propionate (final OD600 were adversely affected).

TABLE 40

| | | | | | | | mM | | | | |
|---|---|---|---|---|---|---|---|---|---|---|---|
| pESC-His | pESC-Leu | pESC-Trp | pESC-Ura | Media | OD600i | OD600f | Acetone | MEK | EtOH | 1-PrOH | Propionic |
| empty | empty | empty | empty | 2% gal., 0.02% suc | 0.20 | 1.83 | 0.00 | 0.0000 | 89.20 | 0.09 | 0.00 |
| adc-Cb/phaA-Ar | scoAB-Bs | YfiD-Ec/tdbC-Ec | pflBA-Hi | 2% gal., 0.02% suc | 0.20 | 1.59 | 0.28 | 0.0000 | 90.30 | 0.70 | 0.00 |
| adc-Ch/phaA-Ar | scoAB-Bs | fyiD = | pflBA-Ec | 2% gal., 0.02% suc | 0.20 | 1.53 | 0.27 | 0.000 | 85.60 | 0.63 | 0.00 |
| | | Ec/tdbC-Ec | | | | | | | | | |
| adc-Ch/phaA-Ar | scoAB-Hp | yfiD-Ec/tdbC-Ec | pflBA-Ec | 2% gal., 0.02% suc | 0.20 | 1.47 | 0.19 | 0.0000 | 81.50 | 0.55 | 0.00 |
| adc-Ch/phaA-Ar | clfAB-Ca | yflD-Ec/tdbC-Ec | pflBA-Hi | 2% gal., 0.02% suc | 0.20 | 1.71 | 0.14 | 0.0003 | 88.50 | .041 | 0.00 |
| empty | empty | empty | empty | 2% gal., 0.02% suc, | 0.20 | 0.93 | 0.00 | 0.0000 | 59.00 | 0.03 | 39.79 |
| | | | | 1 g/l proprionate | | | | | | | |
| empty | empty | empty | empty | 2% gal., 0.02% suc, | 0.20 | 0.80 | 0.00 | 0.0000 | 53.10 | 0.03 | 61.90 |
| | | | | 1 g/l proprionate | | | | | | | |
| adc-Cb/phaA-Ar | ctfAB-Ca | yflD-Ec/tdbC-Ec | pflBA-Hi | 2% gal., 0.02% suc, | 0.20 | 0.77 | 0.01 | 0.0037 | 54.40 | 0.13 | 62.62 |
| | | | | 1 g/l proprionate | | | | | | | |
| adc-Cb/phaA-Ar | scoAB-Hp | yflD-Ec/tdbC-Ec | pflBA-Ed | 2% gal., 0.02% suc, | 0.20 | 0.71 | 0.02 | 0.0043 | 49.30 | 0.18 | 61.74 |
| | | | | 1 g/l proprionate | | | | | | | |
| adc-Cb/phaA-Ar | ctfAB-Ca | yflD-Ec/tdbC-Ec | pflBA-Hi | 2% gal., 0.02% suc, | 0.20 | 0.87 | 0.01 | 0.0044 | 58.70 | 0.17 | 39.81 |
| | | | | 1 g/l proprionate | | | | | | | |
| adc-Cb/phaA-Ar | scoAB-Hp | yflD-Ec/tdbC-Ec | pflBA-Ec | 2% gal., 0.02% suc, | 0.20 | 0.78 | 0.03 | 0.0058 | 52.70 | 0.22 | 39.15 |
| | | | | 1 g/l proprionate | | | | | | | |
| adc-Cb/phaA-Ar | scoAB-Bs | yflD-Ec/tdbC-Ec | pflBA-Ec | 2% gal., 0.02% suc, | 0.20 | 0.69 | 0.03 | 0.0063 | 48.70 | 0.18 | 62.03 |
| | | | | 1 g/l proprionate | | | | | | | |
| adc-Cb/phaA-Ar | scoAB-Bs | yflD-Ec/tdbC-Ec | pflBA-Hi | 2% gal., 0.02% suc, | 0.20 | 0.74 | 0.03 | 0.0065 | 52.50 | 0.21 | 62.26 |
| | | | | 1 g/l proprionate | | | | | | | |
| adc-Cb/phaA-Ar | scoAB-Bs | yflD-Ec/tdbC-Ec | pflBA-Ec | 2% gal., 0.02% suc, | 0.20 | 0.80 | 0.04 | 0.0079 | 53.50 | 0.24 | 40.12 |
| | | | | 1 g/l proprionate | | | | | | | |
| adc-Cb/phaA-Ar | scoAB-Bs | yflD-Ec/tdbC-Ec | pflBA-Hi | 2% gal., 0.02% suc, | 0.20 | 0.88 | 0.04 | 0.0085 | 58.40 | 0.28 | 40.18 |
| | | | | 1 g/l proprionate | | | | | | | |

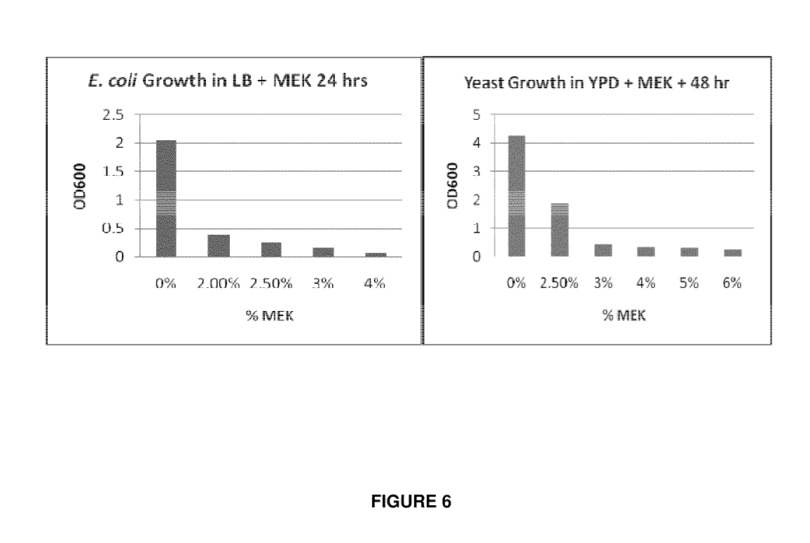

For both E. coli and S. cerevisiae, cells were first grown in rich media+various concentrations of MEK to determine the concentration of MEK they could tolerate and grow. For E. coli cells could “grow” (approximately two doublings) in medium containing 2% MEK, while yeast grew (approximately two doublings) in medium with 2.5% MEK (FIG. 6). Attempts were made to increase tolerance to MEK by serially diluting cells in medium containing the same amount of MEK with and without the mutagen nitrosoguanidine. However, no significant increase in tolerance was obtained in the amount of time this was tested.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A non-naturally occurring microbial organism comprising a microbial organism having a methyl ethyl ketone pathway comprising at least one exogenous nucleic acid encoding a methyl ethyl ketone pathway enzyme expressed in a sufficient amount to produce methyl ethyl ketone, said methyl ethyl ketone pathway comprising a β-ketothiolase, a β-ketovalerate decarboxylase and an enzyme selected from the group consisting of a β-ketovaleryl-CoA hydrolase and a β-ketovaleryl-CoA transferase.

2. The non-naturally occurring microbial organism of claim 1, further comprising a propionyl-CoA pathway comprising at least one exogenous nucleic acid encoding a propionyl-CoA pathway enzyme expressed in a sufficient amount to produce propionyl-CoA.

3. The non-naturally occurring microbial organism of claim 2, wherein said propionyl-CoA pathway enzyme is selected from the group consisting of a PEP carboxylase, a PEP carboxykinase, a pyruvate kinase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

4. The non-naturally occurring microbial organism of claim 2, wherein said propionyl-CoA pathway enzyme comprises a threonine deaminase.

5. The organism of claim 4, wherein said propionyl-CoA pathway enzyme further comprises a pyruvate formate lyase.

6. The organism of claim 4, wherein said propionyl-CoA pathway enzyme further comprises a pyruvate formate lyase activating enzyme.

7. The non-naturally occurring microbial organism of claim 1, further comprising an acetyl-CoA pathway comprising at least one exogenous nucleic acid encoding an acetyl-CoA pathway enzyme expressed in a sufficient amount to produce acetyl-CoA.

8. The non-naturally occurring microbial organism of claim 7, wherein said acetyl-CoA pathway enzyme is selected from the group consisting of a pyruvate kinase, a pyruvate formate lyase, and a formate hydrogen lyase.

9. The non-naturally occurring microbial organism of claim 1, wherein said at least one exogenous nucleic acid is a heterologous nucleic acid.

10. The non-naturally occurring microbial organism of claim 1, wherein said non-naturally occurring microbial organism is in a substantially anaerobic culture medium.

11. The non-naturally occurring microbial organism of claim 1, further comprising a 2-butanol pathway, said 2-butanol pathway comprising at least one exogenous nucleic acid encoding a 2-butanol pathway enzyme expressed in a sufficient amount to produce 2-butanol, said 2-butanol pathway comprising a methyl ethyl ketone reductase.

12. The non-naturally occurring microbial organism of claim 11, further comprising an acetyl-CoA pathway comprising at least one exogenous nucleic acid encoding an acetyl-CoA pathway enzyme expressed in a sufficient amount to produce acetyl-CoA.

13. The non-naturally occurring microbial organism of claim 12, wherein said acetyl-CoA pathway enzyme is selected from the group consisting of a pyruvate dehdyrogenase, a pyruvate ferredoxin oxidoreductase, a pyruvate formate lyase, and a formate dehydrogenase.

14. A non-naturally occurring microbial organism, comprising a microbial organism having a methyl ethyl ketone pathway comprising at least one exogenous nucleic acid encoding a methyl ethyl ketone pathway enzyme expressed in a sufficient amount to produce methyl ethyl ketone, said methyl ethyl ketone pathway comprising a 2-methylacetoacetyl-CoA thiolase, a 2-methylacetoacetate decarboxylase and an enzyme selected from the group consisting of a 2-methylacetoacetyl-CoA hydrolase and a 2-methylacetoacetyl-CoA transferase.

15. The non-naturally occurring microbial organism of claim 14, further comprising a propionyl-CoA pathway comprising at least one exogenous nucleic acid encoding a propionyl-CoA pathway enzyme expressed in a sufficient amount to produce propionyl-CoA.

16. The non-naturally occurring microbial organism of claim 15, wherein said propionyl-CoA pathway enzyme is selected from the group consisting of a PEP carboxylase, a PEP carboxykinase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

17. The non-naturally occurring microbial organism of claim 15, wherein said propionyl-CoA pathway enzyme comprises a threonine deaminase.

18. The organism of claim 17, wherein said propionyl-CoA pathway enzyme further comprises a pyruvate formate lyase.

19. The organism of claim 17, wherein said propionyl-CoA pathway enzyme further comprises a pyruvate formate lyase activating enzyme.

20. The non-naturally occurring microbial organism of claim 14, further comprising an acetyl-CoA pathway comprising at least one exogenous nucleic acid encoding an acetyl-CoA pathway enzyme expressed in a sufficient amount to produce acetyl-CoA.

21. The non-naturally occurring microbial organism of claim 20, wherein said acetyl-CoA pathway enzyme is selected from the group consisting of a pyruvate kinase, a pyruvate formate lyase, a pyruvate dehydrogenase, a pyruvate ferredoxin oxidoreductase, formate dehydrogenase and a formate hydrogen lyase.

22. The non-naturally occurring microbial organism of claim 14, wherein said at least one exogenous nucleic acid is a heterologous nucleic acid.

23. The non-naturally occurring microbial organism of claim 14, wherein said non-naturally occurring microbial organism is in a substantially anaerobic culture medium.

24. The non-naturally occurring microbial organism of claim 14, further comprising a 2-butanol pathway, said 2-butanol pathway comprising at least one exogenous nucleic acid encoding a 2-butanol pathway enzyme expressed in a sufficient amount to produce 2-butanol, said 2-butanol pathway comprising a methyl ethyl ketone reductase.

25. A method for producing methyl ethyl ketone comprising culturing the non-naturally occurring microbial organism of claim 1, under conditions and for a sufficient period of time to produce methyl ethyl ketone.

26. The method of claim 25, further comprising a propionyl-CoA pathway comprising at least one exogenous nucleic acid encoding a propionyl-CoA pathway enzyme expressed in a sufficient amount to produce propionyl-CoA.

27. The method of claim 26, wherein said propionyl-CoA pathway enzyme is selected from the group consisting of a PEP carboxylase, a PEP carboxykinase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

28. The method of claim 26, wherein said propionyl-CoA pathway enzyme comprises a threonine deaminase.

29. The method of claim 28, wherein said propionyl-CoA pathway enzyme further comprises a pyruvate formate lyase.

30. The method of claim 28, wherein said propionyl-CoA pathway enzyme further comprises a pyruvate formate lyase activating enzyme.

31. The method of claim 25, further comprising an acetyl-CoA pathway comprising at least one exogenous nucleic acid encoding an acetyl-CoA pathway enzyme expressed in a sufficient amount to produce acetyl-CoA.

32. The method of claim 31, wherein said acetyl-CoA pathway enzyme is selected from the group consisting of a pyruvate kinase, a pyruvate formate lyase, a pyruvate dehydrogenase, a pyruvate ferredoxin oxidoreductase, a formate dehydrogenase and a formate hydrogen lyase.

33. The method of claim 25, wherein said non-naturally occurring microbial organism is in a substantially anaerobic culture medium.

34. The method of claim 25, wherein said at least one exogenous nucleic acid is a heterologous nucleic acid.

35. A method for producing 2-BuOH comprising culturing the non-naturally occurring microbial organism of claim 11, under conditions and for a sufficient period of time to produce 2-BuOH.

36. The method of claim 35, further comprising an acetyl-CoA pathway comprising at least one exogenous nucleic acid encoding an acetyl-CoA pathway enzyme expressed in a sufficient amount to produce acetyl-CoA.

37. The method of claim 36, wherein said acetyl-CoA pathway enzyme is selected from the group consisting of a pyruvate dehdyrogenase, a pyruvate ferredoxin oxidoreductase, a pyruvate formate lyase, and a formate dehydrogenase.

38. The method of claim 35, wherein said non-naturally occurring microbial organism is in a substantially anaerobic culture medium.

39. The method of claim 35, wherein said at least one exogenous nucleic acid is a heterologous nucleic acid.

40. A method for producing methyl ethyl ketone comprising culturing the non-naturally occurring microbial organism of claim 14, under conditions and for a sufficient period of time to produce methyl ethyl ketone.

41. The method of claim 40, further comprising a propionyl-CoA pathway comprising at least one exogenous nucleic acid encoding a propionyl-CoA pathway enzyme expressed in a sufficient amount to produce propionyl-CoA.

42. The method of claim 41, wherein said propionyl-CoA pathway enzyme is selected from the group consisting of a PEP carboxylase, a PEP carboxykinase, a pyruvate carboxylase, a methylmalonyl-CoA carboxytransferase, a malate dehydrogenase, a fumarase, a fumarate reductase, a succinyl-CoA transferase, a succinyl-CoA synthetase, a methylmalonyl-CoA mutase, a methylmalonyl-CoA epimerase, a methylmalonyl-CoA decarboxylase, and a methylmalonyl-CoA carboxytransferase.

43. The method of claim 41, wherein said propionyl-CoA pathway enzyme comprises a threonine deaminase.

44. The method of claim 43, wherein said propionyl-CoA pathway enzyme further comprises a pyruvate formate lyase

45. The method of claim 43, wherein said propionyl-CoA pathway enzyme further comprises a pyruvate formate lyase activating enzyme.

46. The method of claim 40, further comprising an acetyl-CoA pathway comprising at least one exogenous nucleic acid encoding an acetyl-CoA pathway enzyme expressed in a sufficient amount to produce acetyl-CoA.

47. The method of claim 46, wherein said acetyl-CoA pathway enzyme is selected from the group consisting of a pyruvate kinase, a pyruvate formate lyase, a pyruvate dehydrogenase, a pyruvate ferredoxin oxidoreductase, a formate dehydrogenase and formate hydrogen lyase.

48. The method of claim 40, wherein said non-naturally occurring microbial organism is in a substantially anaerobic culture medium.

49. The method of claim 40, wherein said at least one exogenous nucleic acid is a heterologous nucleic acid.

50. A method for producing 2-BuOH comprising culturing the non-naturally occurring microbial organism of claim 24, under conditions and for a sufficient period of time to produce 2-BuOH.

51. The method of claim 50, wherein said non-naturally occurring microbial organism is in a substantially anaerobic culture medium.

52. The method of claim 50, wherein said at least one exogenous nucleic acid is a heterologous nucleic acid.
